SEQUENCE LISTING (323 sequences)

SEQ ID NO: 1 - DNA, 1680 bp, Artificial Sequence
  Synthetic Sequence
ccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccaaaaggccggcggccacgaaaaaggccggccaggcaaaaaagaaaaag

SEQ ID NO: 2 - DNA, 3009 bp, Artificial Sequence
  Synthetic Sequence
ccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 3 - RNA, 10 nt, Artificial Sequence
  Synthetic Sequence
uuuuuuuuuu

SEQ ID NO: 4 - RNA, 10 nt, Artificial Sequence
  Synthetic Sequence
uuuuauuuuu

SEQ ID NO: 5 - RNA, 11 nt, Artificial Sequence
  Synthetic Sequence
uuuuauuuuuu

SEQ ID NO: 6 - RNA, 12 nt, Artificial Sequence
  Synthetic Sequence
uuuuauuuuuuu

SEQ ID NO: 7 - RNA, 13 nt, Artificial Sequence
  Synthetic Sequence
uuuuauuuuuuuu

SEQ ID NO: 8 - RNA, 14 nt, Artificial Sequence
  Synthetic Sequence
uuuuauuuuuuuuu

SEQ ID NO: 9 - RNA, 15 nt, Artificial Sequence
  Synthetic Sequence
uuuuauuuuuuuuuu

SEQ ID NO: 10 - RNA, 10 nt, Artificial Sequence
  Synthetic Sequence
uuuauuuauu

SEQ ID NO: 11 - RNA, 11 nt, Artificial Sequence
  Synthetic Sequence
uuuauuuauuu

SEQ ID NO: 12 - RNA, 12 nt, Artificial Sequence
  Synthetic Sequence
uuuauuuauuuu

SEQ ID NO: 13 - RNA, 13 nt, Artificial Sequence
  Synthetic Sequence
uuuauuuauuuuu

SEQ ID NO: 14 - RNA, 14 nt, Artificial Sequence
  Synthetic Sequence
uuuauuuauuuuuu

SEQ ID NO: 15 - RNA, 10 nt, Artificial Sequence
  Synthetic Sequence
uuuuauuuua

SEQ ID NO: 16 - RNA, 11 nt, Artificial Sequence
  Synthetic Sequence
uuuuauuuuau

SEQ ID NO: 17 - RNA, 12 nt, Artificial Sequence
  Synthetic Sequence
uuuuauuuuauu

SEQ ID NO: 18 - RNA, 13 nt, Artificial Sequence
  Synthetic Sequence
uuuuauuuuauuu

SEQ ID NO: 19 - RNA, 14 nt, Artificial Sequence
  Synthetic Sequence
uuuuauuuuauuuu

SEQ ID NO: 20 - RNA, 15 nt, Artificial Sequence
  Synthetic Sequence
uuuuauuuuauuuuu

SEQ ID NO: 21 - RNA, 16 nt, Artificial Sequence
  Synthetic Sequence
uuuuauuuuauuuuuu

SEQ ID NO: 22 - RNA, 10 nt, Artificial Sequence
  Synthetic Sequence
uuuuguuuuu

SEQ ID NO: 23 - RNA, 11 nt, Artificial Sequence
  Synthetic Sequence
uuuuguuuuuu

SEQ ID NO: 24 - RNA, 12 nt, Artificial Sequence
  Synthetic Sequence
uuuuguuuuuuu

SEQ ID NO: 25 - RNA, 13 nt, Artificial Sequence
  Synthetic Sequence
uuuuguuuuuuuu

SEQ ID NO: 26 - RNA, 14 nt, Artificial Sequence
  Synthetic Sequence
uuuuguuuuuuuuu

SEQ ID NO: 27 - RNA, 15 nt, Artificial Sequence
  Synthetic Sequence
uuuuguuuuuuuuuu

SEQ ID NO: 28 - RNA, 10 nt, Artificial Sequence
  Synthetic Sequence
uuuguuuguu

SEQ ID NO: 29 - RNA, 11 nt, Artificial Sequence
  Synthetic Sequence
uuuguuuguuu

SEQ ID NO: 30 - RNA, 12 nt, Artificial Sequence
  Synthetic Sequence
uuuguuuguuuu

SEQ ID NO: 31 - RNA, 13 nt, Artificial Sequence
  Synthetic Sequence
uuuguuuguuuuu

SEQ ID NO: 32 - RNA, 14 nt, Artificial Sequence
  Synthetic Sequence
uuuguuuguuuuuu

SEQ ID NO: 33 - RNA, 10 nt, Artificial Sequence
  Synthetic Sequence
uuuuguuuug

SEQ ID NO: 34 - RNA, 11 nt, Artificial Sequence
  Synthetic Sequence
uuuuguuuugu

SEQ ID NO: 35 - RNA, 12 nt, Artificial Sequence
  Synthetic Sequence
uuuuguuuuguu

SEQ ID NO: 36 - RNA, 13 nt, Artificial Sequence
  Synthetic Sequence
uuuuguuuuguuu

SEQ ID NO: 37 - RNA, 14 nt, Artificial Sequence
  Synthetic Sequence
uuuuguuuuguuuu

SEQ ID NO: 38 - RNA, 15 nt, Artificial Sequence
  Synthetic Sequence
uuuuguuuuguuuuu

SEQ ID NO: 39 - RNA, 16 nt, Artificial Sequence
  Synthetic Sequence
uuuuguuuuguuuuuu

SEQ ID NO: 40 - RNA, 10 nt, Artificial Sequence
  Synthetic Sequence
uuuucuuuuu

SEQ ID NO: 41 - RNA, 11 nt, Artificial Sequence
  Synthetic Sequence
uuuucuuuuuu

SEQ ID NO: 42 - RNA, 12 nt, Artificial Sequence
  Synthetic Sequence
uuuucuuuuuuu

SEQ ID NO: 43 - RNA, 13 nt, Artificial Sequence
  Synthetic Sequence
uuuucuuuuuuuu

SEQ ID NO: 44 - RNA, 14 nt, Artificial Sequence
  Synthetic Sequence
uuuucuuuuuuuuu

SEQ ID NO: 45 - RNA, 15 nt, Artificial Sequence
  Synthetic Sequence
uuuucuuuuuuuuuu

SEQ ID NO: 46 - RNA, 10 nt, Artificial Sequence
  Synthetic Sequence
uuucuuucuu

SEQ ID NO: 47 - RNA, 11 nt, Artificial Sequence
  Synthetic Sequence
uuucuuucuuu

SEQ ID NO: 48 - RNA, 12 nt, Artificial Sequence
  Synthetic Sequence
uuucuuucuuuu

SEQ ID NO: 49 - RNA, 13 nt, Artificial Sequence
  Synthetic Sequence
uuucuuucuuuuu

SEQ ID NO: 50 - RNA, 14 nt, Artificial Sequence
  Synthetic Sequence
uuucuuucuuuuuu

SEQ ID NO: 51 - RNA, 10 nt, Artificial Sequence
  Synthetic Sequence
uuuucuuuuc

SEQ ID NO: 52 - RNA, 11 nt, Artificial Sequence
  Synthetic Sequence
uuuucuuuucu

SEQ ID NO: 53 - RNA, 12 nt, Artificial Sequence
  Synthetic Sequence
uuuucuuuucuu

SEQ ID NO: 54 - RNA, 13 nt, Artificial Sequence
  Synthetic Sequence
uuuucuuuucuuu

SEQ ID NO: 55 - RNA, 14 nt, Artificial Sequence
  Synthetic Sequence
uuuucuuuucuuuu

SEQ ID NO: 56 - RNA, 15 nt, Artificial Sequence
  Synthetic Sequence
uuuucuuuucuuuuu

SEQ ID NO: 57 - RNA, 16 nt, Artificial Sequence
  Synthetic Sequence
uuuucuuuucuuuuuu

SEQ ID NO: 58 - RNA, 37 nt, Artificial Sequence
  Synthetic Sequence
guugcagaacccgaauagacgaaugaaggaaugcaac

SEQ ID NO: 59 - DNA, 37 bp, Artificial Sequence
  Synthetic Sequence
gttgcagaacccgaatagacgaatgaaggaatgcaac

SEQ ID NO: 60 - RNA, 161 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaa

SEQ ID NO: 61 - DNA, 161 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaa

SEQ ID NO: 62 - DNA, 20 bp, Artificial Sequence
  Synthetic Sequence
cacgtctcatatgccccttg

SEQ ID NO: 63 - DNA, 20 bp, Artificial Sequence
  Synthetic Sequence
cacacacacagtgggctacc

SEQ ID NO: 64 - DNA, 20 bp, Artificial Sequence
  Synthetic Sequence
gacgtgggagacacacacac

SEQ ID NO: 65 - DNA, 20 bp, Artificial Sequence
  Synthetic Sequence
catccccaggacacacacac

SEQ ID NO: 66 - DNA, 20 bp, Artificial Sequence
  Synthetic Sequence
agcacatgtgcacacacaca

SEQ ID NO: 67 - DNA, 20 bp, Artificial Sequence
  Synthetic Sequence
agaacacatacccctgggcc

SEQ ID NO: 68 - RNA, 20 nt, Artificial Sequence
  Synthetic Sequence
cacgucucauaugccccuug

SEQ ID NO: 69 - RNA, 20 nt, Artificial Sequence
  Synthetic Sequence
cacacacacagugggcuacc

SEQ ID NO: 70 - RNA, 20 nt, Artificial Sequence
  Synthetic Sequence
gacgugggagacacacacac

SEQ ID NO: 71 - RNA, 20 nt, Artificial Sequence
  Synthetic Sequence
cauccccaggacacacacac

SEQ ID NO: 72 - RNA, 20 nt, Artificial Sequence
  Synthetic Sequence
agcacaugugcacacacaca

SEQ ID NO: 73 - RNA, 20 nt, Artificial Sequence
  Synthetic Sequence
agaacacauaccccugggcc

SEQ ID NO: 74 - RNA, 202 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaac

SEQ ID NO: 75 - RNA, 222 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacgucucauaugccccuug

SEQ ID NO: 76 - RNA, 233 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacgucucauaugccccuuguuuuauuuuuu

SEQ ID NO: 77 - RNA, 222 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuacc

SEQ ID NO: 78 - RNA, 223 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccu

SEQ ID NO: 79 - RNA, 224 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuu

SEQ ID NO: 80 - RNA, 225 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuu

SEQ ID NO: 81 - RNA, 226 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuu

SEQ ID NO: 82 - RNA, 227 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuu

SEQ ID NO: 83 - RNA, 228 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuuu

SEQ ID NO: 84 - RNA, 227 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuua

SEQ ID NO: 85 - RNA, 228 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuau

SEQ ID NO: 86 - RNA, 229 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauu

SEQ ID NO: 87 - RNA, 230 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuu

SEQ ID NO: 88 - RNA, 231 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuu

SEQ ID NO: 89 - RNA, 232 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuuu

SEQ ID NO: 90 - RNA, 233 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuuuu

SEQ ID NO: 91 - RNA, 234 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuuuuu

SEQ ID NO: 92 - RNA, 235 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuuuuuu

SEQ ID NO: 93 - RNA, 236 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuuuuuuu

SEQ ID NO: 94 - RNA, 237 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuuuuuuuu

SEQ ID NO: 95 - RNA, 232 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuauuuuuu

SEQ ID NO: 96 - RNA, 236 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuauuuauuuuuu

SEQ ID NO: 97 - RNA, 57 nt, Artificial Sequence
  Synthetic Sequence
guugcagaacccgaauagacgaaugaaggaaugcaaccacacacacagugggcuacc

SEQ ID NO: 98 - RNA, 68 nt, Artificial Sequence
  Synthetic Sequence
guugcagaacccgaauagacgaaugaaggaaugcaaccacacacacagugggcuaccuuuuauuuuuu

SEQ ID NO: 99 - RNA, 231 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuauuuuauuuuuu

SEQ ID NO: 100 - RNA, 232 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuacuuuuauuuuuu

SEQ ID NO: 101 - RNA, 234 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccauuuuauuuuuu

SEQ ID NO: 102 - RNA, 238 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccauuuauuuuauuuuuu

SEQ ID NO: 103 - RNA, 243 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccauuuaaggaguuuuauuuuuu

SEQ ID NO: 104 - RNA, 222 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgacgugggagacacacacac

SEQ ID NO: 105 - RNA, 233 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgacgugggagacacacacacuuuuauuuuuu

SEQ ID NO: 106 - RNA, 222 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacac

SEQ ID NO: 107 - RNA, 233 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacacuuuuauuuuuu

SEQ ID NO: 108 - RNA, 228 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacacuuuuuu

SEQ ID NO: 109 - RNA, 232 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacacuuuauuuuuu

SEQ ID NO: 110 - RNA, 236 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacacuuuauuuauuuuuu

SEQ ID NO: 111 - RNA, 57 nt, Artificial Sequence
  Synthetic Sequence
guugcagaacccgaauagacgaaugaaggaaugcaaccauccccaggacacacacac

SEQ ID NO: 112 - RNA, 68 nt, Artificial Sequence
  Synthetic Sequence
guugcagaacccgaauagacgaaugaaggaaugcaaccauccccaggacacacacacuuuuauuuuuu

SEQ ID NO: 113 - RNA, 230 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacauuuuauuuuuu

SEQ ID NO: 114 - RNA, 230 nt, Artificial Sequence; identical to SEQ ID NO: 113
  Synthetic Sequence

SEQ ID NO: 115 - RNA, 232 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacauuuuauuuuuu

SEQ ID NO: 116 - RNA, 234 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacacauuuuauuuuuu

SEQ ID NO: 117 - RNA, 238 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacacauuucuuuuauuuuuu

SEQ ID NO: 118 - RNA, 243 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccauccccaggacacacacacauuucaaaaguuuuauuuuuu

SEQ ID NO: 119 - RNA, 222 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacagcacaugugcacacacaca

SEQ ID NO: 120 - RNA, 233 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacagcacaugugcacacacacauuuuauuuuuu

SEQ ID NO: 121 - RNA, 222 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacagaacacauaccccugggcc

SEQ ID NO: 122 - RNA, 228 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacagaacacauaccccugggccuuuuuu

SEQ ID NO: 123 - RNA, 233 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacagaacacauaccccugggccuuuuauuuuuu

SEQ ID NO: 124 - RNA, 57 nt, Artificial Sequence
  Synthetic Sequence
guugcagaacccgaauagacgaaugaaggaaugcaacagaacacauaccccugggcc

SEQ ID NO: 125 - RNA, 68 nt, Artificial Sequence
  Synthetic Sequence
guugcagaacccgaauagacgaaugaaggaaugcaacagaacacauaccccugggccuuuuauuuuuu

SEQ ID NO: 126 - DNA, 202 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaac

SEQ ID NO: 127 - DNA, 222 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacgtctcatatgccccttg

SEQ ID NO: 128 - DNA, 233 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacgtctcatatgccccttgttttatttttt

SEQ ID NO: 129 - DNA, 222 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacc

SEQ ID NO: 130 - DNA, 223 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacct

SEQ ID NO: 131 - DNA, 224 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacctt

SEQ ID NO: 132 - DNA, 225 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttt

SEQ ID NO: 133 - DNA, 226 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacctttt

SEQ ID NO: 134 - DNA, 227 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttt

SEQ ID NO: 135 - DNA, 228 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacctttttt

SEQ ID NO: 136 - DNA, 227 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacctttta

SEQ ID NO: 137 - DNA, 228 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttat

SEQ ID NO: 138 - DNA, 229 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttatt

SEQ ID NO: 139 - DNA, 230 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttattt

SEQ ID NO: 140 - DNA, 231 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttatttt

SEQ ID NO: 141 - DNA, 232 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttattttt

SEQ ID NO: 142 - DNA, 232 bp, Artificial Sequence; identical to SEQ ID NO: 141
  Synthetic Sequence

SEQ ID NO: 143 - DNA, 234 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttattttttt

SEQ ID NO: 144 - DNA, 235 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttatttttttt

SEQ ID NO: 145 - DNA, 236 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttattttttttt

SEQ ID NO: 146 - DNA, 237 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttatttttttttt

SEQ ID NO: 147 - DNA, 231 bp, Artificial Sequence
  Synthetic Sequence
ttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacctttatttttt

SEQ ID NO: 148 - DNA, 236 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacctttatttatttttt

SEQ ID NO: 149 - DNA, 57 bp, Artificial Sequence
  Synthetic Sequence
gttgcagaacccgaatagacgaatgaaggaatgcaaccacacacacagtgggctacc

SEQ ID NO: 150 - DNA, 68 bp, Artificial Sequence
  Synthetic Sequence
gttgcagaacccgaatagacgaatgaaggaatgcaaccacacacacagtgggctaccttttatttttt

SEQ ID NO: 151 - DNA, 231 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctattttatttttt

SEQ ID NO: 152 - DNA, 232 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctacttttatttttt

SEQ ID NO: 153 - DNA, 234 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccattttatttttt

SEQ ID NO: 154 - DNA, 238 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccatttattttatttttt

SEQ ID NO: 155 - DNA, 243 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccatttaaggagttttatttttt

SEQ ID NO: 156 - DNA, 222 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacgacgtgggagacacacacac

SEQ ID NO: 157 - DNA, 233 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacgacgtgggagacacacacacttttatttttt

SEQ ID NO: 158 - DNA, 222 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacac

SEQ ID NO: 159 - DNA, 233 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacacttttatttttt

SEQ ID NO: 160 - DNA, 228 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacactttttt

SEQ ID NO: 161 - DNA, 232 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacactttatttttt

SEQ ID NO: 162 - DNA, 236 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacactttatttatttttt

SEQ ID NO: 163 - DNA, 57 bp, Artificial Sequence
  Synthetic Sequence
gttgcagaacccgaatagacgaatgaaggaatgcaaccatccccaggacacacacac

SEQ ID NO: 164 - DNA, 68 bp, Artificial Sequence
  Synthetic Sequence
gttgcagaacccgaatagacgaatgaaggaatgcaaccatccccaggacacacacacttttatttttt

SEQ ID NO: 165 - DNA, 230 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacattttatttttt

SEQ ID NO: 166 - DNA, 231 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacttttatttttt

SEQ ID NO: 167 - DNA, 232 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacattttatttttt

SEQ ID NO: 168 - DNA, 234 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacacattttatttttt

SEQ ID NO: 169 - DNA, 238 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacacatttcttttatttttt

SEQ ID NO: 170 - DNA, 243 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccatccccaggacacacacacatttcaaaagttttatttttt

SEQ ID NO: 171 - DNA, 222 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacagcacatgtgcacacacaca

SEQ ID NO: 172 - DNA, 233 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacagcacatgtgcacacacacattttatttttt

SEQ ID NO: 173 - DNA, 222 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacagaacacatacccctgggcc

SEQ ID NO: 174 - DNA, 228 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacagaacacatacccctgggcctttttt

SEQ ID NO: 175 - DNA, 233 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacagaacacatacccctgggccttttatttttt

SEQ ID NO: 176 - DNA, 57 bp, Artificial Sequence
  Synthetic Sequence
gttgcagaacccgaatagacgaatgaaggaatgcaacagaacacatacccctgggcc

SEQ ID NO: 177 - DNA, 68 bp, Artificial Sequence
  Synthetic Sequence
gttgcagaacccgaatagacgaatgaaggaatgcaacagaacacatacccctgggccttttatttttt

SEQ ID NO: 178 - DNA, 22 bp, Artificial Sequence
  Synthetic Sequence
gccaacatacagaagtcaggaa

SEQ ID NO: 179 - DNA, 20 bp, Artificial Sequence
  Synthetic Sequence
ccaggccatttctcagggtt

SEQ ID NO: 180 - DNA, 20 bp, Artificial Sequence
  Synthetic Sequence
gtacagcttcccagactccg

SEQ ID NO: 181 - DNA, 20 bp, Artificial Sequence
  Synthetic Sequence
tgctccaggaacctcaccta

SEQ ID NO: 182 - DNA, 20 bp, Artificial Sequence
  Synthetic Sequence
gccttcccccacctttcttt

SEQ ID NO: 183 - DNA, 21 bp, Artificial Sequence
  Synthetic Sequence
gagggcctatttcccatgatt

SEQ ID NO: 184 - DNA, 21 bp, Artificial Sequence
  Synthetic Sequence
gagggcctatttcccatgatt

SEQ ID NO: 185 - DNA, 20 bp, Artificial Sequence
  Synthetic Sequence
tgtgcagacaaacggaactc

SEQ ID NO: 186 - DNA, 31 bp, Artificial Sequence
  Synthetic Sequence
aaaaaataaaatgtgcagacaaacggaactc

SEQ ID NO: 187 - DNA, 45 bp, Artificial Sequence
  Synthetic Sequence
ggtagcccactgtgtgtgtggttgcattccttcattcgtctattc

SEQ ID NO: 188 - DNA, 46 bp, Artificial Sequence
  Synthetic Sequence
aggtagcccactgtgtgtgtggttgcattccttcattcgtctattc

SEQ ID NO: 189 - DNA, 47 bp, Artificial Sequence
  Synthetic Sequence
aaggtagcccactgtgtgtgtggttgcattccttcattcgtctattc

SEQ ID NO: 190 - DNA, 48 bp, Artificial Sequence
  Synthetic Sequence
aaaggtagcccactgtgtgtgtggttgcattccttcattcgtctattc

SEQ ID NO: 191 - DNA, 49 bp, Artificial Sequence
  Synthetic Sequence
aaaaggtagcccactgtgtgtgtggttgcattccttcattcgtctattc

SEQ ID NO: 192 - DNA, 50 bp, Artificial Sequence
  Synthetic Sequence
aaaaaggtagcccactgtgtgtgtggttgcattccttcattcgtctattc

SEQ ID NO: 193 - DNA, 51 bp, Artificial Sequence
  Synthetic Sequence
aaaaaaggtagcccactgtgtgtgtggttgcattccttcattcgtctattc

SEQ ID NO: 194 - DNA, 50 bp, Artificial Sequence
  Synthetic Sequence
taaaaggtagcccactgtgtgtgtggttgcattccttcattcgtctattc

SEQ ID NO: 195 - DNA, 51 bp, Artificial Sequence
  Synthetic Sequence
ataaaaggtagcccactgtgtgtgtggttgcattccttcattcgtctattc

SEQ ID NO: 196 - DNA, 52 bp, Artificial Sequence
  Synthetic Sequence
aataaaaggtagcccactgtgtgtgtggttgcattccttcattcgtctattc

SEQ ID NO: 197 - DNA, 53 bp, Artificial Sequence
  Synthetic Sequence
aaataaaaggtagcccactgtgtgtgtggttgcattccttcattcgtctattc

SEQ ID NO: 198 - DNA, 54 bp, Artificial Sequence
  Synthetic Sequence
aaaataaaaggtagcccactgtgtgtgtggttgcattccttcattcgtctattc

SEQ ID NO: 199 - DNA, 55 bp, Artificial Sequence
  Synthetic Sequence
aaaaataaaaggtagcccactgtgtgtgtggttgcattccttcattcgtctattc

SEQ ID NO: 200 - DNA, 56 bp, Artificial Sequence
  Synthetic Sequence
aaaaaataaaaggtagcccactgtgtgtgtggttgcattccttcattcgtctattc

SEQ ID NO: 201 - DNA, 57 bp, Artificial Sequence
  Synthetic Sequence
aaaaaaataaaaggtagcccactgtgtgtgtggttgcattccttcattcgtctattc

SEQ ID NO: 202 - DNA, 58 bp, Artificial Sequence
  Synthetic Sequence
aaaaaaaataaaaggtagcccactgtgtgtgtggttgcattccttcattcgtctattc

SEQ ID NO: 203 - DNA, 59 bp, Artificial Sequence
  Synthetic Sequence
aaaaaaaaataaaaggtagcccactgtgtgtgtggttgcattccttcattcgtctattc

SEQ ID NO: 204 - DNA, 60 bp, Artificial Sequence
  Synthetic Sequence
aaaaaaaaaataaaaggtagcccactgtgtgtgtggttgcattccttcattcgtctattc

SEQ ID NO: 205 - DNA, 55 bp, Artificial Sequence
  Synthetic Sequence
aaaaaataaaggtagcccactgtgtgtgtggttgcattccttcattcgtctattc

SEQ ID NO: 206 - DNA, 59 bp, Artificial Sequence
  Synthetic Sequence
aaaaaataaataaaggtagcccactgtgtgtgtggttgcattccttcattcgtctattc

SEQ ID NO: 207 - DNA, 45 bp, Artificial Sequence; identical to SEQ ID NO: 187
  Synthetic Sequence

SEQ ID NO: 208 - DNA, 56 bp, Artificial Sequence; identical to SEQ ID NO: 200
  Synthetic Sequence

SEQ ID NO: 209 - DNA, 40 bp, Artificial Sequence
  Synthetic Sequence
aaaaaataaaatagcccactgtgtgtgtggttgcattcct

SEQ ID NO: 210 - DNA, 41 bp, Artificial Sequence
  Synthetic Sequence
aaaaaataaaagtagcccactgtgtgtgtggttgcattcct

SEQ ID NO: 211 - DNA, 43 bp, Artificial Sequence
  Synthetic Sequence
aaaaaataaaatggtagcccactgtgtgtgtggttgcattcct

SEQ ID NO: 212 - DNA, 47 bp, Artificial Sequence
  Synthetic Sequence
aaaaaataaaataaatggtagcccactgtgtgtgtggttgcattcct

SEQ ID NO: 213 - DNA, 52 bp, Artificial Sequence
  Synthetic Sequence
aaaaaataaaactccttaaatggtagcccactgtgtgtgtggttgcattcct

SEQ ID NO: 214 - DNA, 45 bp, Artificial Sequence
  Synthetic Sequence
gtgtgtgtgtctcccacgtcgttgcattccttcattcgtctattc

SEQ ID NO: 215 - DNA, 55 bp, Artificial Sequence
  Synthetic Sequence
aaaaaataaaatgtgtgtgtctcccacgtcgttgcattccttcattcgtctattc

SEQ ID NO: 216 - DNA, 45 bp, Artificial Sequence
  Synthetic Sequence
gtgtgtgtgtcctggggatggttgcattccttcattcgtctattc

SEQ ID NO: 217 - DNA, 55 bp, Artificial Sequence
  Synthetic Sequence
aaaaaataaaatgtgtgtgtcctggggatggttgcattccttcattcgtctattc

SEQ ID NO: 218 - DNA, 50 bp, Artificial Sequence
  Synthetic Sequence
aaaaaatgtgtgtgtcctggggatggttgcattccttcattcgtctattc

SEQ ID NO: 219 - DNA, 54 bp, Artificial Sequence
  Synthetic Sequence
aaaaaataaatgtgtgtgtcctggggatggttgcattccttcattcgtctattc

SEQ ID NO: 220 - DNA, 58 bp, Artificial Sequence
  Synthetic Sequence
aaaaaataaataaatgtgtgtgtcctggggatggttgcattccttcattcgtctattc

SEQ ID NO: 221 - DNA, 45 bp, Artificial Sequence; identical to SEQ ID NO: 216
  Synthetic Sequence

SEQ ID NO: 222 - DNA, 56 bp, Artificial Sequence
  Synthetic Sequence
aaaaataaaagtgtgtgtgtcctggggatggttgcattccttcattcgtcatattc

SEQ ID NO: 223 - DNA, 39 bp, Artificial Sequence
  Synthetic Sequence
aaaaaataaaatgtgtgtcctggggatggttgcattcct

SEQ ID NO: 224 - DNA, 40 bp, Artificial Sequence
  Synthetic Sequence
aaaaaataaaagtgtgtgtcctggggatggttgcattcct

SEQ ID NO: 225 - DNA, 41 bp, Artificial Sequence
  Synthetic Sequence
aaaaaataaaatgtgtgtgtcctggggatggttgcattcct

SEQ ID NO: 226 - DNA, 43 bp, Artificial Sequence
  Synthetic Sequence
aaaaaataaaatgtgtgtgtgtcctggggatggttgcattcct

SEQ ID NO: 227 - DNA, 47 bp, Artificial Sequence
  Synthetic Sequence
aaaaaataaaagaaatgtgtgtgtgtcctggggatggttgcattcct

SEQ ID NO: 228 - DNA, 52 bp, Artificial Sequence
  Synthetic Sequence
aaaaaataaaacttttgaaatgtgtgtgtgtcctggggatggttgcattcct

SEQ ID NO: 229 - DNA, 45 bp, Artificial Sequence
  Synthetic Sequence
tgtgtgtgtgcacatgtgctgttgcattccttcattcgtctattc

SEQ ID NO: 230 - DNA, 55 bp, Artificial Sequence
  Synthetic Sequence
aaaaaataaaagtgtgtgtgcacatgtgctgttgcattccttcattcgtctattc

SEQ ID NO: 231 - DNA, 31 bp, Artificial Sequence
  Synthetic Sequence
ggcccaggggtatgtgttctgttgcattcct

SEQ ID NO: 232 - DNA, 37 bp, Artificial Sequence
  Synthetic Sequence
aaaaaaggcccaggggtatgtgttctgttgcattcct

SEQ ID NO: 233 - DNA, 42 bp, Artificial Sequence
  Synthetic Sequence
aaaaaataaaaggcccaggggtatgtgttctgttgcattcct

SEQ ID NO: 234 - DNA, 31 bp, Artificial Sequence; identical to SEQ ID NO: 231
  Synthetic Sequence

SEQ ID NO: 235 - DNA, 42 bp, Artificial Sequence; identical to SEQ ID NO: 233
  Synthetic Sequence

SEQ ID NO: 236 - DNA, 20 bp, Artificial Sequence
  Synthetic Sequence
ttgtgcagaattggagagga

SEQ ID NO: 237 - protein, 7 aa, Artificial Sequence
  Synthetic Sequence
PKKKRKV

SEQ ID NO: 238 - protein, 16 aa, Artificial Sequence
  Synthetic Sequence
KRPAATKKAGQAKKKK

SEQ ID NO: 239 - protein, 9 aa, Artificial Sequence
  Synthetic Sequence
PAAKRVKLD

SEQ ID NO: 240 - protein, 11 aa, Artificial Sequence
  Synthetic Sequence
RQRRNELKRSP

SEQ ID NO: 241 - protein, 38 aa, Artificial Sequence
  Synthetic Sequence
NQSSNFGPMKGGNFGGRSSGPYGGGGQYFAKPRNQGGY

SEQ ID NO: 242 - protein, 42 aa, Artificial Sequence
  Synthetic Sequence
RMRIZFKNKGKDTAELRRRRVEVSVELRKAKKDEQILKRRNV

SEQ ID NO: 243 - protein, 8 aa, Artificial Sequence
  Synthetic Sequence
VSRKRPRP

SEQ ID NO: 244 - protein, 8 aa, Artificial Sequence
  Synthetic Sequence
PPKKARED

SEQ ID NO: 245 - protein, 8 aa, Artificial Sequence
  Synthetic Sequence
PQPKKKPL

SEQ ID NO: 246 - protein, 12 aa, Artificial Sequence
  Synthetic Sequence
SALIKKKKKMAP

SEQ ID NO: 247 - protein, 5 aa, Artificial Sequence
  Synthetic Sequence
DRLRR

SEQ ID NO: 248 - protein, 7 aa, Artificial Sequence
  Synthetic Sequence
PKQKKRK

SEQ ID NO: 249 - protein, 10 aa, Artificial Sequence
  Synthetic Sequence
RKLKKKIKKL

SEQ ID NO: 250 - protein, 10 aa, Artificial Sequence
  Synthetic Sequence
REKKKFLKRR

SEQ ID NO: 251 - protein, 20 aa, Artificial Sequence
  Synthetic Sequence
KRKGDEVDGVDEVAKKKSKK

SEQ ID NO: 252 - protein, 17 aa, Artificial Sequence
  Synthetic Sequence
RKCLQAGMNLEARKTKK

SEQ ID NO: 253 - RNA, 69 nt, Artificial Sequence
  Synthetic Sequence
uucauuuuuccucuccaauucugcacaagaaaguugcagaacccgaauagacgaaugaaggaaugcaac

SEQ ID NO: 254 - DNA, 69 bp, Artificial Sequence
  Synthetic Sequence
ttcatttttcctctccaattctgcacaagaaaguugcagaacccgaauagacgaaugaaggaaugcaac

SEQ ID NO: 255 - DNA, 37 bp, Artificial Sequence; identical to SEQ ID NO: 59
  Synthetic Sequence

SEQ ID NO: 256 - DNA, 233 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttattttat

SEQ ID NO: 257 - DNA, 234 bp, Artificial Sequence
  Synthetic Sequence
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaaccacacacacagtgggctaccttttattttatt

SEQ ID NO: 258 - DNA, 56 bp, Artificial Sequence
  Synthetic Sequence
ataaaataaaaggtagcccactgtgtgtgtggttgcattccttcattcgtctattc

SEQ ID NO: 259 - DNA, 57 bp, Artificial Sequence
  Synthetic Sequence
aataaaataaaaggtagcccactgtgtgtgtggttgcattccttcattcgtctattc

SEQ ID NO: 260 - DNA, 20 bp, Artificial Sequence
  Synthetic Sequence
gaatacccccattcttcagg

SEQ ID NO: 261 - RNA, 222 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagg

SEQ ID NO: 262 - RNA, 223 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucaggu

SEQ ID NO: 263 - RNA, 224 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguu

SEQ ID NO: 264 - RNA, 225 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuu

SEQ ID NO: 265 - RNA, 226 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuu

SEQ ID NO: 266 - RNA, 227 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuu

SEQ ID NO: 267 - RNA, 228 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuuu

SEQ ID NO: 268 - RNA, 227 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuua

SEQ ID NO: 269 - RNA, 228 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuau

SEQ ID NO: 270 - RNA, 229 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauu

SEQ ID NO: 271 - RNA, 230 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauuu

SEQ ID NO: 272 - RNA, 231 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauuuu

SEQ ID NO: 273 - RNA, 232 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauuuuu

SEQ ID NO: 274 - RNA, 233 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauuuuuu

SEQ ID NO: 275 - RNA, 234 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauuuuuuu

SEQ ID NO: 276 - RNA, 235 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauuuuuuuu

SEQ ID NO: 277 - RNA, 236 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauuuuuuuuu

SEQ ID NO: 278 - RNA, 237 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacgaauacccccauucuucagguuuuauuuuuuuuuu

SEQ ID NO: 279 - RNA, 233 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuuau

SEQ ID NO: 280 - RNA, 234 nt, Artificial Sequence
  Synthetic Sequence
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaaccacacacacagugggcuaccuuuuauuuuauu

SEQ ID NO: 281 - protein, 560 aa, Artificial Sequence
  Synthetic Sequence
PKKKRKVGIHGVPAAMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPKRPAATKKAGQAKKKK

SEQ ID NO: 282 - protein, 497 aa, Artificial Sequence
  Synthetic Sequence
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 283 - protein, 422 aa, Artificial Sequence
  Synthetic Sequence
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 284 - DNA, 1491 bp, Artificial Sequence
  Synthetic Sequence
atgggcgagagcgttaaagcaatcaaactgaagatcctggacatgttcctggacccggagtgcactaaacaggacgataactggcgtaaagacctgtctaccatgtctcgtttctgcgcagaggcgggtaacatgtgcctgcgcgacctgtacaattacttttctatgccgaaagaagaccgtattagctccaaggatctgtacaacgccatgtaccacaaaactaaactgctgcacccggaactgccgggtaaagtggcgaaccagatcgtcaaccacgctaaagatgtgtggaaacgcaacgcgaagctgatctatcgtaatcagatctctatgccgacttacaagatcaccaccgcaccgattcgcctgcaaaacaacatctataaactgatcaaaaacaagaacaaatatatcatcgatgtccagctgtacagcaaagaatactctaaagacagcggcaaaggcacccaccgctacttcctggttgcagtgcgcgattctagcacgcgtatgatcttcgaccgcatcatgagcaaagaccacatcgatagctccaaatcctacacccagggtcagctgcagattaaaaaagaccatcagggcaaatggtactgtatcattccgtacaccttcccgactcatgagaccgtcctggatccggacaaagttatgggtgtggacctgggcgtggctaaagcagtgtattgggcgttcaactcctcttacaaacgcggttgtatcgatggtggtgaaattgaacacttccgcaaaatgatccgtgctcgtcgtgtctccattcagaaccagattaaacattctggcgacgcgcgcaagggtcatggtcgtaaacgtgcactgaagccgattgaaaccctgtctgaaaaagaaaaaaacttccgcgatactatcaaccatcgttacgcaaaccgtatcgtggaggcagcgattaagcagggctgcggcaccatccagatcgaaaatctggagggtatcgccgatactacgggttccaaatttctgaaaaactggccatactatgacctgcagaccaaaatcgtgaacaaagcgaaggaacatggcatcaccgtcgttgcgatcaacccgcagtatacttctcaacgttgctccatgtgtggttacattgaaaaaaccaaccgttcctctcaggcagtgttcgagtgcaaacagtgcggctatggttcccgtaccatctgcatcaactgccgccacgttcaggtaagcggtgatgtctgcgaagaatgcggcggcatcgttaaaaaagaaaacgttaacgcggactataacgcggcgaaaaacatctccaccccatatatcgatcagatcatcatggagaaatgtctggagctgggtatcccgtaccgtagcatcacttgcaaagaatgcggtcatatccaggcgtctggtaacacttgtgaggtgtgcggttccaccaacattctgaaaccgaaaaaaatccgtaaagctaaa

SEQ ID NO: 285 - DNA, 1266 bp, Artificial Sequence
  Synthetic Sequence
atgatcaaggtttatcgctatgaaattgtgaaaccgctggacctggactggaaagagttcggtactatcctgcgtcagctgcagcaggaaacgcgtttcgcactgaacaaagcgacgcagctggcttgggaatggatgggcttcagctctgattacaaagacaaccacggcgaataccctaaatccaaagatatcctgggttacaccaacgtgcacggttatgcgtaccacaccatcaaaactaaagcgtaccgcctgaactccggtaatctgtctcagaccatcaagcgtgcgaccgaccgtttcaaagcgtaccagaaggaaatcctgcgcggtgacatgagcatcccgtcctataagcgtgacatcccgctggatctgatcaaagaaaacatcagcgtaaaccgcatgaaccacggtgactacatcgcctccctgtctctgctgtctaacccggcaaagcaagagatgaacgttaaacgcaaaatctccgttatcattatcgttcgtggcgcaggcaaaaccatcatggatcgtattctgtccggtgaatatcaagtgtccgcaagccagatcatccatgacgatcgtaaaaataaatggtatctgaacatctcttatgatttcgaaccgcagacccgcgtactggacctgaacaaaatcatgggtatcgatctgggcgttgcagtagcggtgtacatggctttccagcacaccccggcacgctacaaactggaaggtggtgagatcgaaaacttccgtcgtcaagtggaatctcgtcgcatctctatgctgcgtcagggtaaatatgcaggtggtgcgcgtggtggtcatggtcgtgacaaacgtattaaaccaatcgaacagctgcgcgataaaatcgccaacttccgtgataccaccaaccaccgctactctcgttatatcgttgatatggcaatcaaagagggttgtggtactatccagatggaagacctgacgaacatccgcgatatcggttctcgtttcctgcagaactggacctactacgatctgcagcagaaaattatctacaaagcggaggaagcaggtatcaaagtcattaaaatcgatccgcagtatactagccagcgctgcagcgaatgtggtaacattgatagcggcaaccgcattggtcaggcaatctttaaatgccgcgcttgtggctacgaagcgaacgcagactacaacgcggcgcgtaacatcgccatcccgaatattgacaaaatcattgccgaatctattaaa

SEQ ID NO: 286 - DNA, 20 bp, Artificial Sequence
  Synthetic Sequence
ggggtgaccgccggagcgcg

SEQ ID NO: 287 - DNA, 161 bp, Artificial Sequence; identical to SEQ ID NO: 61
  Synthetic Sequence

SEQ ID NO: 288 - DNA, 32 bp, Artificial Sequence
  Synthetic Sequence
gtcgcatcttgcgtaagcgcgtggattgaaac

SEQ ID NO: 289 - DNA, 29 bp, Artificial Sequence
  Synthetic Sequence
gtttgcgagctagcttgtggagtgtgaac

SEQ ID NO: 290 - DNA, 52 bp, Artificial Sequence
  Synthetic Sequence
gtcgcatcttgcgtaagcgcgtggattgaaaccacacacacagtgggctacc

SEQ ID NO: 291 - DNA, 58 bp, Artificial Sequence
  Synthetic Sequence
gtcgcatcttgcgtaagcgcgtggattgaaaccacacacacagtgggctacctttttt

SEQ ID NO: 292 - DNA, 63 bp, Artificial Sequence
  Synthetic Sequence
gtcgcatcttgcgtaagcgcgtggattgaaaccacacacacagtgggctaccttttatttttt

SEQ ID NO: 293 - DNA, 52 bp, Artificial Sequence
  Synthetic Sequence
gtcgcatcttgcgtaagcgcgtggattgaaacggggtgaccgccggagcgcg

SEQ ID NO: 294 - DNA, 58 bp, Artificial Sequence
  Synthetic Sequence
gtcgcatcttgcgtaagcgcgtggattgaaacggggtgaccgccggagcgcgtttttt

SEQ ID NO: 295 - DNA, 63 bp, Artificial Sequence
  Synthetic Sequence
gtcgcatcttgcgtaagcgcgtggattgaaacggggtgaccgccggagcgcgttttatttttt

SEQ ID NO: 296 - DNA, 49 bp, Artificial Sequence
  Synthetic Sequence
gtttgcgagctagcttgtggagtgtgaaccacacacacagtgggctacc

SEQ ID NO: 297 - DNA, 55 bp, Artificial Sequence
  Synthetic Sequence
gtttgcgagctagcttgtggagtgtgaaccacacacacagtgggctacctttttt

SEQ ID NO: 298 - DNA, 60 bp, Artificial Sequence
  Synthetic Sequence
gtttgcgagctagcttgtggagtgtgaaccacacacacagtgggctaccttttatttttt

SEQ ID NO: 299 - DNA, 49 bp, Artificial Sequence
  Synthetic Sequence
gtttgcgagctagcttgtggagtgtgaacggggtgaccgccggagcgcg

SEQ ID NO: 300 - DNA, 55 bp, Artificial Sequence
  Synthetic Sequence
gtttgcgagctagcttgtggagtgtgaacggggtgaccgccggagcgcgtttttt

SEQ ID NO: 301 - DNA, 60 bp, Artificial Sequence
  Synthetic Sequence
gtttgcgagctagcttgtggagtgtgaacggggtgaccgccggagcgcgttttatttttt

SEQ ID NO: 302 - DNA, 20 bp, Artificial Sequence
  Synthetic Sequence
ttgtgcagaattggagagga

SEQ ID NO: 303 - DNA, 31 bp, Artificial Sequence
  Synthetic Sequence
ggtagcccactgtgtgtgtggtttcaatcca

SEQ ID NO: 304 - DNA, 37 bp, Artificial Sequence
  Synthetic Sequence
aaaaaaggtagcccactgtgtgtgtggtttcaatcca

SEQ ID NO: 305 - DNA, 42 bp, Artificial Sequence
  Synthetic Sequence
aaaaaataaaaggtagcccactgtgtgtgtggtttcaatcca

SEQ ID NO: 306 - DNA, 31 bp, Artificial Sequence
  Synthetic Sequence
cgcgctccggcggtcaccccgtttcaatcca

SEQ ID NO: 307 - DNA, 37 bp, Artificial Sequence
  Synthetic Sequence
aaaaaacgcgctccggcggtcaccccgtttcaatcca

SEQ ID NO: 308 - DNA, 42 bp, Artificial Sequence
  Synthetic Sequence
aaaaaataaaacgcgctccggcggtcaccccgtttcaatcca

SEQ ID NO: 309 - DNA, 31 bp, Artificial Sequence
  Synthetic Sequence
ggtagcccactgtgtgtgtggttcacactcc

SEQ ID NO: 310 - DNA, 37 bp, Artificial Sequence
  Synthetic Sequence
aaaaaaggtagcccactgtgtgtgtggttcacactcc

SEQ ID NO: 311 - DNA, 42 bp, Artificial Sequence
  Synthetic Sequence
aaaaaataaaaggtagcccactgtgtgtgtggttcacactcc

SEQ ID NO: 312 - DNA, 31 bp, Artificial Sequence
  Synthetic Sequence
cgcgctccggcggtcaccccgttcacactcc

SEQ ID NO: 313 - DNA, 37 bp, Artificial Sequence
  Synthetic Sequence
aaaaaacgcgctccggcggtcaccccgttcacactcc

SEQ ID NO: 314 - DNA, 42 bp, Artificial Sequence
  Synthetic Sequence
aaaaaataaaacgcgctccggcggtcaccccgttcacactcc

SEQ ID NO: 315 - DNA, 81 bp, Artificial Sequence
  Synthetic Sequence
tacccatacgatgttccagattacgcttatccctacgacgtgcctgattatgcatacccatatgatgtccccgactatgcc

SEQ ID NO: 316 - DNA, 3009 bp, Artificial Sequence; identical to SEQ ID NO: 2
  Synthetic Sequence

SEQ ID NO: 317 - protein, 1003 aa, Artificial Sequence
  Synthetic Sequence
PKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 318 - DNA, 3009 bp, Artificial Sequence
  Synthetic Sequence
ccaaagaagaagcggaaagtcatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccctctggaggatctagcggaggatcctctggcagcgagacaccaggaacaagcgagtcagcaacaccagagagcagtggcggcagcagcggcggcagctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 319 - protein, 1003 aa, Artificial Sequence
  Synthetic Sequence
PKKKRKVMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPSGGSSGGSSGSETPGTSESATPESSGGSSGGSSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 320 - DNA, 2823 bp, Artificial Sequence
  Synthetic Sequence
atgtccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgactctggtggttcttctggtggttctagcggcagcgagactcccgggacctcagagtccgccacacccgaaagttctggtggttcttctggtggttcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgactctggtggttcttctggtggttctagcggcagcgagactcccgggacctcagagtccgccacacccgaaagttctggtggttcttctggtggttctgccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccaaaaggccggcggccacgaaaaaggccggccaggcaaaaaagaaaaag

SEQ ID NO: 321 - protein, 941 aa, Artificial Sequence
  Synthetic Sequence
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPKRPAATKKAGQAKKKK

SEQ ID NO: 322 - DNA, 2823 bp, Artificial Sequence
  Synthetic Sequence
atggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccctctggaggatctagcggaggatcctctggcagcgagacaccaggaacaagcgagtcagcaacaccagagagcagtggcggcagcagcggcggcagctccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgactctggtggttcttctggtggttctagcggcagcgagactcccgggacctcagagtccgccacacccgaaagttcaggtggatcttcaggtggatcttcggaagtggaattttcgcacgagtattggatgaggcacgctttaactctcgctaagagagcacgagacgaacgggaagtgccggttggggctgtcctcgtactcaataatcgagttatcggagaaggctggaacagggcaatcggactccacgatcccacagctcatgccgagataatggcgcttcgacaaggaggcctagtcatgcaaaattatcgtcttattgacgcgaccctctacgtgacctttgagccatgcgttatgtgtgcgggtgcaatgatacattcccggataggacgtgtagtatttggagttcgcaacgcgaagaccggtgcggctggttctctcatggatgtcctgcactaccctgggatgaatcaccgcgttgaaatcactgaaggcattttggccgatgaatgcgcggccctgttatgttacttttttcgcatgcccaggcaggtctttaacgcacagaagaaagcccaatcgtccactgataaaaggccggcggccacgaaaaaggccggccaggcaaaaaagaaaaag

SEQ ID NO: 323 - protein, 941 aa, Artificial Sequence
  Synthetic Sequence
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDKRPAATKKAGQAKKKK

(19) United States
(12) Patent Application Publication
(10) Pub. No.: US 2022/0307018 A1
(43) Pub. Date: Sep. 29, 2022

(54) ENGINEERED GUIDE RNA FOR THE OPTIMIZED CRISPR/Cas12f1 SYSTEM AND USE THEREOF

(71) Applicant: GENKORE INC, Daejeon (KR)

(72) Inventors: Yong Sam KIM, Daejeon (KR); Do Yon KIM, Daejeon (KR); Jeong Heon KO, Daejeon (KR)

(21) Appl. No.: 17/707,254

(22) Filed: Mar. 29, 2022

Related U.S. Application Data
(63) Continuation-in-part of application No. PCT/KR2020/014961, filed on Oct. 29, 2020.

(30) Foreign Application Priority Data
Oct. 29, 2019 (KR) 10-2019-0135935

(51) Int. Cl. C12N 15/11; C12N 9/22; C12N 15/87; C12N 15/90

(52) U.S. Cl. CPC C12N 15/11; C12N 9/22; C12N 15/87; C12N 15/907; C12N 2310/20; C12N 2800/80; C12N 2750/14143

(57) ABSTRACT

Provided are an engineered CRISPR/Cas12f1 system of which editing efficiency is improved compared to a canonical CRISPR/Cas12f1 system, and a use thereof. A U-rich tail sequence in crRNA is provided that can be introduced into an original guide RNA to improve the editing efficiency of the CRISPR/Cas12f1 system. The U-rich tail sequence has abundant uridine residues at the 3′-ternimus of crRNA, and may further include additional nucleic acids in addition to uridine depending on the environment in which the guide RNA is actually used and the environment in which the guide RNA is expressed. The inventors of the present application described the structure of the U-rich tail sequence, the position of introduction, and the effect thereof with respect to genome editing efficiency.

### CROSS-REFERENCE TO RELATED APPLICATIONS

This application is a U.S. Continuation-In-Part Application based on International Application No. PCT/KR2020/014961, filed on Oct. 29, 2020, and claims priority to, and the benefit of, Korean Patent Application No. 10-2019-0135935, filed on Oct. 29, 2019, the contents of which are incorporated herein by reference in their entireties.

### DESCRIPTION OF THE TEXT FILE SUBMITTED ELECTRONICALLY

The instant application contains a Sequence Listing which has been submitted in ASCII format via EFS-Web and is hereby incorporated by reference in its entirety. Said ASCII copy, created on or about Jun. 12, 2022, is named “P210098US_SequenceList.txt” and is about 163,840 bytes in size.

### TECHNICAL FIELD

The present disclosure relates to a technique for an area in which a CRISPR/Cas system, particularly, a CRISPR/Cas12f1 system is used for gene editing.

### BACKGROUND ART

A CRISPR/Cas12f1 system is a miniature CRISPR/Cas system which is classified as Class 2, Type V-F. A previous study (Harrington et al., Programmed DNA destruction by CRISPR-Cas14 enzymes, Science 362, 839-842 (2018)) has reported the CRISPR system as CRISPR/Cas14a which is an Archaea-derived CRISPR/Cas system for the first time. Since then, a subsequent study (Karvelis et al., Nucleic Acids Research, Vol. 48, No. 9 5017 (2020)) classified the CRISPR/Cas14 system into a CRISPR/Cas12f system. The CRISPR/Cas12f system belongs to a V-F system which is one subtype among the CRISPR/Cas systems which are classified into the Class 2, Type V, and includes a CRISPR/Cas12f1 system having Cas12f1 as an effector protein. The CRISPR/Cas12f1 system is characterized by the compact size of an effector protein compared to that of CRISPR/Cas9 system. However, as revealed in the previous study (Harington et al., Programmed DNA destruction by miniature CRISPR-Cas14 enzymes, Science 362, 839-842 (2018), US 2020/0190494 A1), the CRISPR/Cas12f1 system, particularly, the CRISPR/Cas14a system exhibits the ability to cleave single-stranded DNA, but has no or extremely low cleavage activity for double-stranded DNA, so that its application to gene editing technology is not feasible.

### DISCLOSURE

### Technical Problem

An aspect of the present disclosure provides an engineered CRISPR/Cas12f1 system with increased gene-editing efficiency.

An aspect of the present disclosure provides an engineered CRISPR/Cas12f1 system having a uridine (U)-rich tail sequence.

An aspect of the present disclosure provides engineered crRNA having a U-rich tail sequence for an engineered CRISPR/Cas12f1 system.

An aspect of the present disclosure provides engineered guide RNA having a U-rich tail sequence for an engineered CRISPR/Cas12f1 system.

An aspect of the present disclosure provides an engineered CRISPR/Cas12f1 complex having a U-rich tail sequence for an engineered CRISPR/Cas12f1 system.

An aspect of the present disclosure provides a vector having a nucleic acid sequence encoding each component of an engineered CRISPR/Cas12f1 system.

An aspect of the present disclosure provides a gene editing method using the engineered CRISPR/Cas12f1 system.

An aspect of the present disclosure provides the use of the engineered CRISPR/Cas12f1 system.

### Technical Solution

In an embodiment, an aspect of the present disclosure provides an engineered CRISPR RNA (crRNA) for a CRISPR/Cas12f1 system, comprising:

a CRISPR RNA repeat sequence;

a spacer sequence which is complementary to a target sequence; and

a U-rich tail sequence which is linked to 3′-terminus of the spacer sequence,

wherein the U-rich tail sequence is represented by (UₐN)ₙU_{b},

N is one of adenosine (A), uridine (U), cytidine (C), and guanosine (G),

a is an integer between 1 to 4, n is an integer selected from 0, 1, and 2, and b is an integer between 1 to 10, and

the engineered CRISPR RNA includes the CRISPR RNA repeat sequence, the spacer sequence, and the U-rich tail sequence which are linked sequentially in the 5′-to-3′ direction.

In an embodiment, an aspect of the present disclosure provides a DNA having a sequence encoding the engineered crRNA.

In an embodiment, the U-rich tail sequence may be UUUUUU, UUUUAUUUUUU, or UUUUGUUUUUU.

In an embodiment, the CRISPR RNA repeat sequence may be the sequence of SEQ ID NO: 58.

In an embodiment, an aspect of the present disclosure provides an engineered guide RNA of a CRISPR/Cas12f1 system, comprising:

a tracrRNA sequence;

a crRNA sequence including a CRISPR RNA repeat sequence, and a spacer sequence which is complementary to a target sequence; and

a U-rich tail sequence which is linked to 3′-terminus of the spacer sequence,

wherein the U-rich tail sequence is represented by (UₐN)ₙU_{b},

N is one of adenosine (A), uridine (U), cytidine (C), and guanosine (G),

a is an integer between 1 to 4 inclusive, n is an integer selected from 0, 1, and 2, and b is an integer between 1 to 10 inclusive.

In an embodiment, the engineered guide RNA further includes a linker sequence, through which the tracrRNA sequence and the crRNA sequence are linked.

In an embodiment, the linker sequence may be 5′-gaaa-3′.

In an embodiment, the U-rich tail sequence may be UUUUUU, UUUUAUUUUUU, or UUUUGUUUUUU.

In an embodiment, an aspect of the present disclosure provides a DNA having a sequence encoding the engineered guide RNA.

In an embodiment, an aspect of the present disclosure provides an engineered CRISPR/Cas12f1 complex, including:

a Cas12f1 protein belonging to the type V-F CRISPR system; and

an engineered guide RNA including: a scaffold sequence which interacts with the Cas12f1 protein; a spacer sequence which is complementary to the target sequence; and

a U-rich tail sequence,

wherein the U-rich tail sequence is represented by (UₐN)ₙU_{b},

N is one of adenosine (A), uridine (U), cytidine (C), and guanosine (G), and

a is an integer between 1 to 4 inclusive, n is an integer selected from 0, 1, and 2, and b is an integer between 1 to 10 inclusive.

In an embodiment, an aspect of the present disclosure provides a vector for expressing an engineered CRISPR/Cas12f1 complex, the vector including:

a first sequence including a sequence that encodes a Cas12f1 protein;

a first promoter sequence operably linked to the first sequence;

a second sequence including a sequence that encodes an engineered guide RNA,

wherein the engineered guide RNA has a scaffold sequence which interacts with the Cas12f1 protein, a spacer sequence which is complementary to a target sequence, and a U-rich tail sequence linked to the 3′-terminus of the spacer sequence,

the U-rich tail sequence is represented by (UₐN)ₙU_{b},

N is one of adenosine (A), uridine (U), cytidine (C), and guanosine (G), and

a is an integer between 1 to 4 inclusive, n is an integer selected from 0, 1, and 2, and b is an integer between 1 to 10 inclusive; and

a second promoter sequence operably linked to the second sequence;

wherein the vector is constructed to express the Cas12f1 protein and the engineered guide RNA,

and thus, the Cas12f1 protein and the engineered guide sequence can form the CRISPR/Cas12f1 complex in a cell, and

the nucleic acid can be edited by the CRISPR/Cas12f1 complex.

In an embodiment, the CRISPR/Cas12f1 system, the CRISPR/Cas12f1 complex, and the vector for expressing an engineered CRISPR/Cas12f1 complex are capable of editing the nucleic acid.

In an embodiment, the nucleic acid includes a target sequence or near a target sequence.

In an embodiment, the second promoter sequence may be a U6 promoter sequence.

In an embodiment, the vector may be a plasmid vector.

In an embodiment, the vector may be a viral vector.

In an embodiment, the vector may be one or more selected from the group consisting of a retrovirus, a lentivirus, an adenovirus, an adeno-associated virus (AAV), a vaccinia virus, a poxvirus and a herpes simplex virus.

In an embodiment, the vector may be a linear PCR amplicon.

An aspect of the present disclosure provides a vector for expressing an engineered CRISPR/Cas12f1 complex, the vector including:

a first sequence including a sequence that encodes a Cas12f1 protein;

a first promoter sequence operably linked to the first sequence;

a second sequence including a sequence that encodes a first engineered guide RNA,

wherein the first engineered guide RNA has a first scaffold sequence which interacts with the Cas12f1 protein, a first spacer sequence which is complementary to a first target sequence, and a first U-rich tail sequence, wherein

the first U-rich tail sequence is represented by (UₐN)ₙU_{b},

N is one of adenosine (A), uridine (U), cytidine (C), and guanosine (G), and

a is an integer between 1 to 4 inclusive, n is an integer selected from 0, 1, and 2, and b is an integer between 1 to 10 inclusive;

a second promoter sequence operably linked to the second sequence;

a third sequence including a sequence that encodes a second engineered guide RNA,

wherein the second engineered guide RNA has a second scaffold sequence which may interact with the Cas12f1 protein, a second spacer sequence which is complementary to a second target sequence, and a second U-rich tail sequence,

the second U-rich tail sequence is represented by (UₐN)ₙU_{b},

N is one of adenosine (A), uridine (U), cytidine (C), and guanosine (G), and

a is an integer between 1 to 4 inclusive, n is an integer selected from 0, 1, and 2, and b is an integer between 1 to 10 inclusive; and

a third promoter sequence operably linked to the third sequence.

In an embodiment, the second promoter sequence and the third promoter sequence may be the same promoter sequence.

In an embodiment, the second promoter sequence may be a H1 promoter sequence, and the third promoter sequence may be a U6 promoter sequence.

In an embodiment, an aspect of the present disclosure provides a method for editing a nucleic acid, in a cell, the method includes:

delivering a Cas12f1 protein or a nucleic acid encoding the Cas12f1 protein, and an engineered guide RNA or a nucleic acid encoding the engineered guide RNA, into the cell,

so that a CRISPR/Cas12f1 complex can be formed in the cell, and

the nucleic acid can be edited by the CRISPR/Cas12f1 complex,

wherein the engineered guide RNA has a scaffold sequence which interacts with the Cas12f1 protein, a spacer sequence which is complementary to a target sequence, and a U-rich tail sequence linked to the 3′-terminus of the spacer sequence,

wherein the U-rich tail sequence is represented by (UₐN)ₙU_{b},

N is one of adenosine (A), uridine (U), cytidine (C), and guanosine (G), and

a is an integer between 1 to 4 inclusive, n is an integer selected from 0, 1, and 2, and b is an integer between 1 to 10 inclusive.

In an embodiment, the delivery can be achieved by introducing the CRISPR/Cas12f1 ribonucleoprotein complex comprising Cas12f1 protein and an engineered gRNA into cells.

In an embodiment, the delivery can be achieved by introducing a vector including the nucleic acid encoding the Cas12f1 protein and an engineered guide RNA into cells.

In an embodiment, the vector may be selected from the group consisting of a retrovirus, a lentivirus, an adenovirus, an adeno-associated virus (AAV), a vaccinia virus, a poxvirus, and a herpes simplex virus.

In an embodiment, an aspect of the present disclosure provides a method for editing a nucleic acid, in the cell, the method including:

bringing a CRISPR/Cas12f1 complex in contact with the nucleic acid,

wherein, the CRISPR/Cas12f1 complex includes a Cas12f1 protein and an engineered guide RNA,

the engineered guide RNA includes a scaffold sequence which interacts with the Cas12f1 protein, a spacer sequence which is complementary to a target sequence, and a U-rich tail sequence,

the U-rich tail sequence is represented by (UₐN)ₙU_{b},

N is one of adenosine (A), uridine (U), cytidine (C), and guanosine (G), and

wherein a is an integer between 1 to 4 inclusive, n is an integer selected from 0, 1, and 2, and b is an integer between 1 to 10 inclusive.

In an embodiment, an aspect of the present disclosure provides a method for targeting a nucleic acid, in a cell, the method includes:

delivering a Cas12f1 protein or a nucleic acid encoding the Cas12f1 protein, and an engineered guide RNA or a nucleic acid encoding the engineered guide RNA, into the cell,

so that a CRISPR/Cas12f1 complex can be formed in the cell, and

the nucleic acid can be targeted by the CRISPR/Cas12f1 complex,

wherein the engineered guide RNA has a scaffold sequence which interacts with the Cas12f1 protein, a spacer sequence which is complementary to a target sequence, and a U-rich tail sequence linked to the 3′-terminus of the spacer sequence,

wherein the U-rich tail sequence is represented by (UₐN)ₙU_{b},

N is one of adenosine (A), uridine (U), cytidine (C), and guanosine (G), and

a is an integer between 1 to 4 inclusive, n is an integer selected from 0, 1, and 2, and b is an integer between 1 to 10 inclusive.

In an embodiment, an aspect of the present disclosure provides a method for targeting a nucleic acid, in the cell, the method including:

bringing a CRISPR/Cas12f1 complex in contact with the nucleic acid,

wherein, the CRISPR/Cas12f1 complex includes a Cas12f1 protein and an engineered guide RNA,

the engineered guide RNA includes a scaffold sequence which interacts with the Cas12f1 protein, a spacer sequence which is complementary to a target sequence, and a U-rich tail sequence,

the U-rich tail sequence is represented by (UₐN)ₙU_{b},

N is one of adenosine (A), uridine (U), cytidine (C), and guanosine (G), and

wherein a is an integer between 1 to 4 inclusive, n is an integer selected from 0, 1, and 2, and b is an integer between 1 to 10 inclusive.

### Advantageous Effects

The engineered CRISPR/Cas12f1 system having the U-rich tail sequence provided in the present disclosure exhibits higher gene editing efficiency when used for gene editing than when a wild-type CRISPR/Cas12f1 system is used.

### DESCRIPTION OF DRAWINGS

FIG. 1 shows an example structure of an engineered guide RNA provided in the present disclosure. (a) shows an example of the structure of a dual guide RNA, including a part of tracrRNA and a repeat sequence of CRISPR RNA (crRNA). The complementary sequences bind each other to form a double-stranded RNA. The names of crRNA parts (a CRISPR RNA repeat sequence part, a spacer sequence part, and a U-rich tail sequence part) are illustrated. (b) shows an example of the structure of a single guide RNA, in which the tracrRNA and the crRNA are linked via a linker. The positions of the CRISPR RNA repeat sequence part, the spacer sequence part, and the U-rich tail sequence part are illustrated. Each sequence illustrated in the drawings is an example sequence.

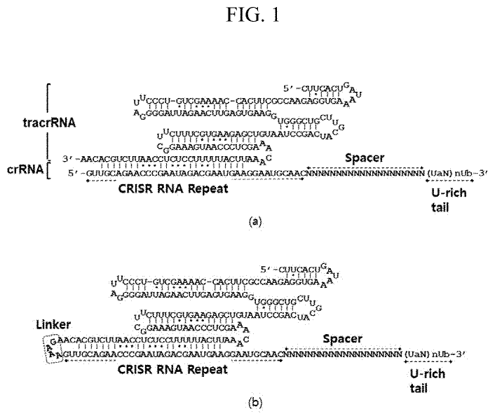

FIG. 2 is a graph showing the results of Experimental Example 3. (a) is a graph showing the indel efficiencies of CRISPR/Cas12f1 system carrying a 3′-U4AU6 U-rich tail crRNA sequence for DYTarget2, DYtarget10 and DYtarget13 in HEK293T cells. In this regard, the indel efficiency of the engineered CRISPR/Cas12f1 system having a U-rich tail sequence is remarkably higher than the indel efficiency of the wild-type CRISPR/Cas12f1 system. (b) is a graph showing the indel efficiencies which the Cas12f1 guide RNA carrying either a U6 or a U4AU6 sequence shows for Intergenic-22 in HEK293T cells.

FIG. 3 is a graph showing the results of Experimental Example 4. It shows the indel efficiencies of either a canonical (wild-type) or an engineered CRISPR/Cas12f1 system with respect to Target 1 (DYtarget2), Target 2 (DYtarget10), Target 3 (Intergenic-22) in HEK293T cells. No crRNA, Canonical gRNA, and gRNA (MS1) refer to the indel efficiency of the crRNA-untreated control, the wild-type CRISPR/Cas12f1 system, and the engineered CRISPR/Cas12f1 system having a crRNA carrying a U4AU6 structure, respectively.

FIG. 4 is a graph showing the results of Experimental Example 7. It shows the indel efficiencies of CRISPR/Cas12f1 systems with different 3′-terminus crRNA sequences with respect to DYtarget2 or DYtarget10 in HEK293 T cells. (a) shows the indel efficiency for DYtarget2 in HEK293 T cells. WT shows the indel efficiency of an experimental group for which sgRNA is not treated. Ux shows the indel efficiency of an experimental group for which no U was not added to the 3′-terminus of spacer sequence of sgRNA. U6, U4AU6, U3AU6, and U3AU3AU6 refer to the 3′-tail sequences of sgRNA.

FIG. 5 is a graph showing the results of Experimental Example 8. It shows the indel efficiency of an engineered CRISPR/Cas12f1 system for DYtarget2 in HEK293 T cells. None shows an indel efficiency of an experimental group for which no U was added to the 3′-terminus of an sgRNA. T, T2, T3, T4, T4A, T4AT, T4AT2, T4AT3, T4AT4, T4AT5, T4AT6, T4AT7, T4AT8, T4AT4AT, and T4AT4AT2 refer to the nucleotide sequences added to the 3′-terminus of a protospacer sequence in an sgRNA-encoding DNA sequence.

FIG. 6 is a graph showing the results of Experimental Example 9. It shows the indel efficiency of CRISPR/Cas12f1 system for CSMD1 in HEK293 T cells. None shows the indel efficiency of an experimental group for which no U was added to the 3′-terminus of an sgRNA. U, U2, U3, U4, U5, U6, U4A, U4AU, U4AU2, U4AU3, U4AU4, U4AU5, U4AU6, U4AU7, U4AU8, U4AU9, and U4AU10 show the indel efficiencies of experimental groups for which an sgRNA shows the corresponding sequence at the 3′-terminus.

FIG. 7 is a graph showing the results of Experimental Example 10. It shows the indel efficiency of CRISPR/Cas12f1 system for DYtarget2 in HEK293 T cells. None refers to the indel efficiency of an experimental group for which no U was added to the 3′-terminus of an sgRNA. T4AT6 and T4GT6 refer to the experimental groups for which T4AT6 and T4GT6 sequence was added to the 3′-terminus of the protospacer sequence of sgRNA-encoding DNA, respectively.

FIG. 8 is a graph showing the results of Experimental Example 11. It shows the indel efficiency of an engineered CRISPR/Cas12f1 system for DYtarget2 and DYtarget10 in HEK293 T cells. (a) indicates the indel efficiencies of the U4AU6 U-rich Cas12f1 sgRNA for DYtarget2 in HEK293 T cells, according to the indicated lengths of a spacer sequence. The T4AT6 sequence was added to the 3′-terminus of the protospacer sequence of sgRNA. In this regards, 18 mer, 19 mer, 20 mer, 21 mer, 25 mer, and 30 mer on the horizontal axis of the graph indicate the length of the spacer sequence of the sgRNAs. The protospacer sequence is shown in Table 25 of Experimental Example 11. (b) indicates the indel efficiencies of the U4AU6 U-rich Cas12f1 sgRNA for DYtarget10 in HEK293 T cells, according to the indicated lengths of a spacer sequence. The T4AT6 sequence was added to the 3′-terminus of the protospacer sequence of sgRNA. In this regard, 17mer, 18 mer, 19 mer, 20 mer, 21 mer, 25 mer, and 30 mer on the horizontal axis of the graph indicate the length of the spacer sequence of the sgRNAs. The protospacer sequence is shown in Table 24 of Experimental Example 11.

FIG. 9 shows the results of western blot analysis using anti-HA antibody for Experimental Example 12-1. In this regard, it was confirmed that AsCas12f1 and SpCas12f1 effector proteins were expressed in HEK293T cells. The AsCas12f1 and SpCas12f1 proteins show a molecular mass of 56.55 kDa and 64.65 kDa, respectively on an SDS-PAGE gel.

### MODES FOR CARRYING OUT THE INVENTION

### Definition of Terms

About

As used herein, the term “about” refers to an amount, a level, a value, a number, a frequency, a percentage, a dimension, a size, a quantity, a weight, or a length that varies to the degree of 30, 25, 20, 25, 10, 9, 8, 7, 6, 5, 4, 3, 2, or 1% with respect to a reference amount, level, value, number, frequency, percentage, dimension, size, quantity, weight, or length.

Operably Linked

As used herein, the term “operably linked” refers to the state of being linked such that in the gene expression technique, a particular component permits another component to function in an intended manner. For example, the case where a promoter sequence is operably linked to a coding sequence means that the promoter is linked so as to affect the intracellular transcription and/or expression of the coding sequence. Further, the term includes all meanings that can be recognized by those skilled in the art, and may be appropriately interpreted according to the context.

Target gene or target nucleic acid

As used herein, “target gene” or “target nucleic acid” basically refers to an intracellular gene or nucleic acid that is targetable by the CRISPR system. The target gene or target nucleic acid may be used interchangeably and may refer to the same subject. The target gene or target nucleic acid may refer to a unique gene or nucleic acid possessed by a target cell, or an externally derived gene or nucleic acid unless otherwise described, and is not particularly limited as long as it may be the target of gene editing. The target gene or target nucleic acid may be single-stranded DNA, double-stranded DNA, and/or RNA. Further, the term includes all meanings that can be recognized by those skilled in the art, and may be appropriately interpreted according to the context.

Target Sequence

As used herein, “target sequence” refers to a particular sequence that a CRISPR/Cas complex recognizes to cleave a target gene or target nucleic acid. The target sequence may be appropriately selected according to the purpose thereof. Specifically, “target sequence” is a sequence included in a target gene or target nucleic acid sequence, and refers to a sequence that is complementary to a spacer sequence included in a guide RNA or an engineered guide RNA provided in the present specification. In general, the spacer sequence is determined in consideration of a target gene or target nucleic acid sequence and a PAM sequence recognized by an effector protein of a CRISPR/Cas system. The target sequence may refer to only a particular strand that complementarily binds to a guide RNA of the CRISPR/Cas complex, and may also refer to the entire target double strand including the particular strand part, which is appropriately interpreted according to the context. Further, the term includes all meanings that can be recognized by those skilled in the art, and may be appropriately interpreted according to the context.

Vector

As used herein, “vector” collectively refers to all materials capable of carrying a genetic material into a cell, unless otherwise specified. For example, a vector may be a DNA molecule including a genetic material of interest, for example, a nucleic acid encoding an effector protein of the CRISPR/Cas system, and/or a nucleic acid encoding a guide RNA, but is not limited thereto. The term includes all meanings that can be recognized by those skilled in the art, and may be appropriately interpreted according to the context.

Engineered

As used herein, “engineered” is a term used to distinguish a material, a molecule, and the like, which have a structure that is already present in nature, and refers to those in which the material, molecule, and the like are artificially modified. For example, the case of “engineered guide RNA” refers to a guide RNA in which the composition of a guide RNA that is present in nature is artificially modified. Further, the term includes all meanings that can be recognized by those skilled in the art, and may be appropriately interpreted according to the context.

Nuclear Localization Sequence, or Signal (NLS)

As used herein, “NLS” refers to a peptide having a certain length, which serves as a type of “tag” by being attached to a protein to be transported, or a sequence thereof, when a material outside the cell nucleus is transported into the nucleus by a nuclear transport action. Specifically, the NLS may be an NLS of SV40 virus large T-antigen having an amino acid sequence PKKKRKV (SEQ ID NO: 237); an NLS from a nucleoplasmin (for example, a nucleoplasmin bipartite NLS having a sequence KRPAATKKAGQAKKKK(SEQ ID NO: 238)); a c-myc NLS having an amino acid sequence PAAKRVKLD (SEQ ID NO: 239) or RQRRNELKRSP (SEQ ID NO: 240); a hRNPA1 M9 NLS having a sequence NQSSNFGPMKGGNFGGRSSGPYGGGGQYFAKPRNQGGY (SEQ ID NO: 241); a sequence RMRIZFKNKGKDTAELRRRRVEVSVELRKAKKDEQILKRRNV (SEQ ID NO: 242) of an IBB domain from importin-alpha; sequences VSRKRPRP (SEQ ID NO: 243) and PPKKARED (SEQ ID NO: 244) of a myoma T protein; a sequence PQPKKKPL (SEQ ID NO: 245) of human p53; a sequence SALI AP (SEQ ID NO: 246) of mouse c-abl IV; sequences DRLRR (SEQ ID NO: 247) and PKQKKRK (SEQ ID NO: 248) of an influenza virus NS1; a sequence RKLKKKIKKL (SEQ ID NO: 249) of a hepatitis virus delta antigen; a sequence REKKKFLKRR (SEQ ID NO: 250) of a mouse Mx1 protein; a sequence KRKGDEVDGVDEVAKKKSKK (SEQ ID NO: 251) of a human poly (ADP-ribose) polymerase; or an NLS sequence derived from a sequence RKCLQAGMNLEARKTKK (SEQ ID NO: 252) of a steroid hormone receptor (human) glucocorticoid, but is not limited thereto. As used herein, the term “NLS” includes all meanings that can be recognized by those skilled in the art, and may be appropriately interpreted according to the context.

Nuclear Export Sequence, or Signal (NES)

As used herein, “NES” refers to a peptide having a certain length, which serves as a type of “tag” by being attached to a protein to be transported, or a sequence thereof, when a material in the cell nucleus is transported outside the nucleus by a nuclear transport action. As used herein, the term “NES” includes all meanings that can be recognized by those skilled in the art, and may be appropriately interpreted according to the context.

Tag

As used herein, “tag” collectively refers to a functional domain added to facilitate the tracking and/or isolation and purification of a peptide or protein. Specifically, the tag includes a tag protein such as a histidine (His) tag, a V5 tag, a FLAG tag, an influenza hemagglutinin (HA) tag, a Myc tag, a VSV-G tag and a thioredoxin (Trx), an auto-fluorescent protein such as a green fluorescent protein (GFP), a yellow fluorescent protein (YFP), a cyan fluorescent protein (CFP), a blue fluorescent protein (BFP), HcRED, and DsRed, and a reporter gene such as glutathione-S-transferase (GST), horseradish peroxidase (HRP), chloramphenicol acetyltransferase (CAT), beta-galactosidase, beta-glucuronidase, and luciferase, but is not limited thereto. As used herein, the term “tag” includes all meanings that can be recognized by those skilled in the art, and may be appropriately interpreted according to the context.

Background Art—CRISPR/Cas12f1 System

Overview

Various types of CRISPR/Cas systems are present in nature, and new CRISPR/Cas systems are still being discovered. Among them, the CRISPR/Cas12f1 system provided in the present disclosure specifically belongs to the Class 2, Type V classification, and is a CRISPR/Cas system belonging to the Cas 14 family (Harrington et al., Programmed DNA destruction by miniature CRISPR-Cas 14 enzymes, Science 362, 839-842 (2018)). Hereinafter, what classification the CRISPR/Cas12f1 system provided in the present disclosure belongs to will be described.

Class 2 CRISPR/Cas System

The CRISPR/Cas system is roughly divided into Class 1 and Class 2. Among them, the Class 2 CRISPR/Cas system is characterized by the effector endonuclease consisting of a large single protein having multiple domains. Among the Class 2 CRISPR/Cas systems, the Type II CRISPR/Cas9 system is a representative CRISPR/Cas system that has been actively studied for gene editing applications. The CRISPR/Cpf1 system is another well-characterized Class 2 system.

Type V CRISPR/Cas System

The Class 2 CRISPR/Cas system is divided into Type II, V, and VI. Among them, the CRISPR/Cas12f1 system provided in the present disclosure belongs to the Type V CRISPR/Cas system. An effector protein of the Type V CRISPR/Cas system is named Cas12, and is named Cas12a, Cas12b, and the like, according to a detailed classification. The Cas12 protein has one nuclease domain (RuvC-like nuclease), which is a feature distinguishing from Type II effector proteins (for example, Cas9 protein) having two nuclease domains (HNH, and RuvC domain). To date, the type V CRISPR/Cas system is divided into 11 subtypes, of which the CRISPR/Cas12f1 system provided in the present disclosure belongs to V-F1, which is a variant of one of the subtypes V-F (Makarova et. al., Nature Reviews, Microbiology volume 18, 67 (2020).

CRISPR/Cas12f1 System

The CRISPR/Cas12f system belongs to the V-F subtype of the type V CRISPR/Cas system, which is further subdivided into variants of V-F1 to V-F3. The CRISPR/Cas12f system includes a CRISPR/Cas14 system including Cas14a, Cas14b, and Cas14c variants among the effector proteins named Cas14 in a previous study (Harrington et al., Programmed DNA destruction by miniature CRISPR-Cas14 enzymes, Science 362, 839-842 (2018)). Among them, the CRISPR/Cas14a system including the Cas14a effector protein is classified as the CRISPR/Cas12f1 system (Makarova et al., Nature Reviews, Microbiology volume 18, 67 (2020)). However, the CRISPR/Cas12f1 includes not only a Cas14a family (Harrington et al., Programmed DNA destruction by miniature CRISPR-Cas14 enzymes, Science 362, 839-842 (2018)), but also a CRISPR/Cas system in which an effector system is named c2c10 (Karvelis et al., Nucleic Acids Research, Vol. 48, No. 9 5017 (2020), Makarova et al., Nature Reviews, Microbiology volume 18, 67 (2020)). Therefore, in the present disclosure, the CRISPR/Cas12f1 system is a concept that includes both the CRISPR/Cas14a system and the CRISPR/c2c10 system unless otherwise described, and among them, when referring to the CRISPR/Cas14a system, it refers to the “CRISPR/Cas14a system” or the “CRISPR/Cas12f1 system belonging to the Cas14 family”. The term has a meaning that those skilled in the art can appropriately interpret according to the context.

Background Art—Design of CRISPR/Cas System Expression Vector

Overview

In order to use a CRISPR/Cas system for gene editing, a method has been widely used, in which each component of the CRISPRCas system is expressed in the cell by introducing a vector having a sequence encoding each component of the CRISPR/Cas system into the cell. Hereinafter, the components of a vector that allow the CRISPR/Cas system to be expressed in the cell will be described.

Nucleic Acid Encoding Component of CRISPR/Cas System

Since the purpose of the vector is to express each component of the CRISPR Cas system in the cell, the sequence of the vector should essentially include one or more of the nucleic acid sequences encoding each component of the CRISPR/Cas system. Specifically, the sequence of the vector includes a nucleic acid sequence encoding a guide RNA, and/or a Cas protein included in a CRISPR/Cas system to be expressed. In this regard, the sequence of the vector may include not only a nucleic acid sequence encoding a wild-type guide RNA and a wild-type Cas protein, but also a nucleic acid sequence encoding an engineered guide RNA and a codon optimized Cas protein, or a nucleic acid sequence encoding an engineered Cas protein, according to the purpose thereof.

Regulatory/Control Component

In order to express the vector in the cell, one or more regulatory/control components should be included. Specifically, the regulatory/control component may include a promoter, an enhancer, an intron, a polyadenylation signal, a Kozak consensus sequence, an internal ribosome entry site (IRES), a splice acceptor, a 2A sequence and/or a replication origin, but is not limited thereto. In this regard, the replication origin may be an f1 replication origin, an SV40 replication origin, a pMB1 replication origin, an adeno replication origin, an AAV replication origin, and/or a BBV replication origin, but is not limited thereto.

Promoter

In order to express an expression target of the vector in the cell, an RNA transcription factor should be activated in the cell by operably linking a promoter sequence to a sequence encoding each component. The promoter sequence may be designed differently depending on the corresponding RNA transcription factor or expression environment, and is not limited as long as the promoter sequence can appropriately express the component of the CRISPR/Cas system in the cell. The promoter sequence may be a promoter that promotes transcription of an RNA polymerase (for example, RNA Pol I, Pol II, or Pol III). For example, the promoter may be one of an SV40 initial promoter, a mouse mammary tumor virus long terminal repeat (LTR) promoter, an adenovirus major late promoter (Ad MLP), a herpes simplex virus (HSV) promoter, a cytomegalovirus (CMV) promoter such as a CMV immediate early promoter region (CMVIE), a Rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), and a human H1 promoter (H1), but is not limited thereto.

Termination Signal

When the vector sequence includes the promoter sequence, the transcription of a sequence operably linked to the promoter is induced by an RNA transcription factor, and a sequence that induces the transcription termination of the RNA transcription factor is called a termination signal. The termination signal may vary depending on the type of promoter sequence. For example, when the promoter is a U6 or H1 promoter, the promoter recognizes a continuous thymidine sequence (for example, a TTTTTT (T6) sequence) as a termination signal.

Additional Expression Component

The vector may include a nucleic acid sequence encoding an additional expression component intended to be expressed by those skilled in the art, if necessary, in addition to the wild-type CRISPR/Cas system configuration and/or the engineered CRISPR/Cas system. For example, the additional expression component may be one of the tags described in the “Tag” section of the <Description of terms> section, but is not limited thereto. For example, the additional expression component may be a herbicide resistance gene such as glyphosate, glufosinate ammonium or phosphinothricin, and an antibiotic resistance gene such as ampicillin, kanamycin, G418, bleomycin, hygromycin, and chloramphenicol, but is not limited thereto.

Form of Expression Vector

The expression vector may be designed in the form of a linear or circular vector.

Limitations of Related Art

The major limitation of the CRISPR/Cas12f1 system, particularly the CRISPR/Cas14a system belonging to the Cas14 family, is that the gene editing activity in the cell does not appear or is too low. In a prior document reported for the first time about the CRISPR/Cas14 system (Harrington et al., Programmed DNA destruction by miniature CRISPR-Cas14 enzymes, Science 362, 839-842 (2018)), prior researchers have found that the CRISPR/Cas14 system exhibits cleavage activity for single-stranded DNA in prokaryotic cells, but fails to exhibit cleavage activity for double-stranded DNA in the cell. In this regard, prior researchers believed that when considering that the CRISPRCas14 system is a system discovered in archaeons, the CRISPR/Cas system in the early stages of evolution shows only cleavage activity for single-stranded DNA, and then has acquired cleavage activity for double-stranded DNA (for example, the CRISPR/Cas9 system) while undergoing evolution. In another prior document (US 2020/0190494 A1), prior researchers have reported the fact that the CRISPR/Cas14 system exhibits cleavage activity for double-stranded DNA in eukaryotic cells, but the cleavage efficiency is 0.1% or less, which appears to be remarkably low. Therefore, according to the prior documents, the CRISPR/Cas12f1 system belonging to the Cas14 family does not show cleavage activity for double-stranded DNA in the cell, or even when the CRISPR/Cas12f1 system shows cleavage activity, the efficiency thereof is very low, so that the CRISPR/Cas12f1 system had a limitation in that it is difficult to actively utilize the CRISPR/Cas12f1 system in gene editing.

U-Rich Tail Sequence

U-Rich Tail Sequence—Overview An aspect of the present disclosure provides a U-rich tail sequence added to the guide RNA to improve the gene editing efficiency of the CRISPR/Cas12f1 system. The

U-rich tail sequence is basically characterized by the presence of a poly-uridinylate sequence, in which one or more uridines are are attached to the 3′-terminus of crRNA. In an embodiment, the U-rich tail sequence may include a sequence of 1 to 10 repeating uridines. The U-rich tail sequence may further include additional nucleotides in addition to multiple Us depending on the actual usage and expression environments of the engineered CRISPR/Cas12f1 system (for example, eukaryotic or prokaryotic internal environment). In an embodiment, the U-rich tail sequence may include a sequence in which one or more of UV, UUV, UUUV, and/or UUUUV are repeated. In this regard, V is any one of adenosine (A), cytidine (C), and guanosine (G). The U-rich tail sequence is added to the 3′-terminus of the Cas12f1 crRNA. In the present disclosure, the U-rich tail sequence serves to increase the indel efficiency of the engineered CRISPR-Cas12f1 system for a target nucleic acid. In this regard, the target nucleic acid may be single-stranded DNA, double-stranded DNA, and/or RNA. The term “U-rich tail sequence” used herein refers to a poly-uridinylate sequence of an sgRNA, or a DNA sequence that encodes the poly-uridinylate sequence and is appropriately interpreted according to the context. In the present disclosure, the structure of the U-rich tail sequence and the effects thereof are described in detail, which will be further described in the following paragraphs.

Background of Introducing U-Rich Tail Sequence—a Related Study on the CRISPR/Cpf1 System

In the previous study, the present inventors have found that when the U-rich tail sequence is linked to the 3′-terminus of the crRNA sequence included in the CRISPR/Cpf1 system, the nucleic acid cleavage efficiency of the CRISPR/Cpf1 system is remarkably increased (Moon et al., Highly efficient genome editing by CRISPR-Cpf1 using CRISPR RNA with a uridinylate-rich 3′-overhang. Nature Commun 9, 3651 (2018)). The present inventors paid attention to the fact that Cpf1 and Cas12f1 all belong to the Type V CRISPR/Cas system (hereinafter, a CRISPR/Cas12 system), and the CRISPR/Cas12 system shares several features including the RuvC domain architecture. Therefore, we explored whether the indel efficiency of the CRISPR/Cas12f1 system is improved by the U-rich tail of crRNA.

Application of the U-Rich Tail Sequence to the CRISPR/Cas12f1 System

The present disclosure provides evidence that, when linked to the 3′-terminus of the Cas12f1 crRNA, the U-rich tail sequence remarkably increases the indel efficiency of the CRISPR/Cas12f1 system. Additionally, various configurations of 3′-terminal sequences of crRNA were investigated with respect to the effects on the indel efficiency, and an optimal structure for the U-rich tail sequence is provided in the present disclosure.

Background of Introducing U-Rich Tail Sequence—not Applicable to all CRISPR/Cas12 Systems

However, as a result of introducing the U-rich tail sequence into other CRISPR/Cas12 systems, it was found that just belonging to the Type V CRISPR/Cas system does not enhance the gene editing efficiency of the CRISPR/Cas12 complex (see Experimental Example 00). It can be inferred that this brings different results for the U-rich tail sequence because even the same Type V group shows different aspects in terms of the presence of tracrRNA, the structure of a Cas protein, and the like. As far as the present inventors know, it has not been clarified by what function and mechanism the U-rich tail sequence added to 3′-terminus of the crRNA increases the nucleic acid cleavage efficiency in the CRISPR Cas system to date, and it is also difficult to determine whether the U-rich tail sequence added to 3′-terminus of the crRNA affects gene cleavage activity based on the structure of the guide RNA and/or Cas protein alone. Therefore, clarifying the role of the U-rich tail sequence and its effect on the CRISPR/Cas system will be a topic for future research. Hereinafter, the U-rich tail sequence introduced into the CRISPR/Cas12f1 system, particularly the system belonging to the Cas14 family will be described in more detail.

U-Rich Tail Sequence Structure—Continuous Uridine Sequence

One of the important points in designing the U-rich tail sequence is to abundantly include a sequence in which one or more uridines are continuous. Through experiments, the present inventors have found that the gene editing efficiency of the CRISPR/Cas12f1 complex was improved when the U-rich tail sequence, which is a sequence in which one or more uridines are continuous, was introduced into the CRISPR/Cas12f1 system. Therefore, the U-rich tail sequence provided in the present disclosure includes a sequence in which one or more uridines are continuous. In an embodiment, the U-rich tail sequence may include a sequence in which 1, 2, 3, 4, 5, 6, 7, 8, 9, 10, 11, 12, 13, 14, 15, 16, 17, 18, 19, or 20 uridines are contiguous.

U-Rich Tail Sequence Structure—Problems that May Occur when a Continuous Uridine Sequence is Included Alone

When the U-rich tail sequence includes a long continuous (for example, a sequence in which 5 or more uridines are continuous) uridine sequence, the following problems may occur when crRNA is expressed by introducing a vector or the like into the cell. When designing a vector expressing the aforementioned continuous uridine sequence, a continuous thymidine sequence corresponding to the continuous uridine sequence is inevitably included in the vector. In this regard, since some (for example, a U6 promoter, and the like) of the promoters used to express the vector in eukaryotic cells use a continuous thymidine sequence, for example, a T6 sequence as a termination signal, the continuous thymidine sequence corresponding to the continuous uridine sequence may be recognized as a termination signal. When the continuous uridine sequence includes 1 to 4 uridines, a sequence of 1 to 4 continuous thymidines is included in the vector, which does not act as a termination signal, so that any problems may occur in expressing the U-rich tail sequence including up to 4 contiguous thymidines. However, when the continuous uridine sequence includes 6 or more uridines, a T6 sequence is included in the vector, which serves as a termination signal, so that a U-rich tail sequence including 5 or more contiguous uridines is not likely to be expressed as intended. Therefore, in order to include abundant uridines in the U-rich tail sequence while using a promoter that recognizes the continuous thymidine sequence as a termination sequence, a structure in which 6 or more uridines are contiguous needs to be avoided.

U-Rich Tail Sequence Structure—Modified Continuous Uridine Sequence

In order to solve the problem as described above, the U-rich tail sequence provided in the present disclosure may include a modified uridine repeat sequence in which one ribonucleoside (A, C, G) is incorporated in every 1 to 5 uridines. In an embodiment, the U-rich tail sequence may include a sequence in which one or more of UV, UUV, UUUV, UUUUV, and/or UUUUUV are repeated. In this regard, the V is one of adenosine (A), cytidine (C), and guanosine (G). The modified continuous uridine sequence is useful in designing a vector expressing an engineered crRNA.

Example of U-Rich Tail Sequence—Uₓ Form

In an embodiment, the U-rich tail sequence may be represented by Uₓ. In an embodiment, x may be 1, 2, 3, 4, 5, 6, 7, 8, 9, 10, 11, 12, 13, 14, 15, 16, 17, 18, 19, or 20. In an embodiment, x may be an integer within the numerical range between two of these numbers. For example, x may be an integer between 1 to 6. In an embodiment, x may be an integer between 1 to 20. In an embodiment, x may be an integer of 20 or more.

Example of U-Rich Tail Sequence—(UₐN)ₙU_{b} Form

In an embodiment, the U-rich tail sequence may be represented by (UₐN)ₙU_{b}. In this regard, N is one of adenosine (A), uridine (U), cytidine (C), and guanosine (G). In this regard, a is an integer between 1 to 5, and n is an integer of 0 or more. In an embodiment, n may be an integer between 0 to 2. In an embodiment, b may be 1, 2, 3, 4, 5, 6, 7, 8, 9, or 10. In an embodiment, b may be an integer within the numerical range between two of these numbers. For example, b may be an integer between 1 to 6.

Example Embodiment of U-Rich Tail Sequence—(UₐV)ₙ Form

In an embodiment, the U-rich tail sequence may be represented by (UₐV)ₙ. In this regard, V is one of adenosine (A), cytidine (C), and guanosine (G). In this regard, a may be an integer between 1 to 4, and n may be an integer of 1 or more.

In an embodiment, the U-rich tail sequence may be represented by (UₐM)ₙ. In this regard, M is adenosine (A), or cytidine (C). In this regard, a may be an integer between 1 to 4, and n may be an integer of 1 or more.

In an embodiment, the U-rich tail sequence may be represented by (UₐR)ₙ. In this regard, R is adenosine (A), or guanosine (G). In this regard, a may be an integer between 1 to 4, and n may be an integer of 1 or more.

In an embodiment, the U-rich tail sequence may be represented by (UₐS)ₙ. In this regard, S is cytidine (C), or guanosine (G). In this regard, a may be an integer between 1 to 4, and n may be an integer of 1 or more.

In an embodiment, the U-rich tail sequence may be represented by (UₐA)ₙ. In this regard, a may be an integer between 1 to 4, and n may be an integer of 1 or more.

In an embodiment, the U-rich tail sequence may be represented by (UₐC)ₙ. In this regard, a may be an integer between 1 to 4, and n may be an integer of 1 or more.

In an embodiment, the U-rich tail sequence may be represented by (UₐC)ₙ. In this regard, a may be an integer between 1 to 4, and n may be an integer of 1 or more.

Example of U-Rich Tail Sequence—(UₐV)ₙU_{b} Form

In an embodiment, the U-rich tail sequence may be represented by (UₐV)ₙU_{b}. In this regard, V is one of adenosine (A), cytidine (C), and guanosine (G). In this regard, a is an integer between 1 to 4, and n is an integer of 0 or more. In an embodiment, n may be 1 or 2. In an embodiment, b may be 1, 2, 3, 4, 5, 6, 7, 8, 9, 10, 11, 12, 13, 14, 15, 16, 17, 18, 19, or 20. In an embodiment, b may be an integer within the numerical range between two of these numbers. For example, b may be an integer between 1 to 6. In an embodiment, b may be an integer between 1 to 20. In an embodiment, b may be an integer of 20 or more.

In an embodiment, the U-rich tail sequence may be represented by (UₐM)ₙ. In this regard, M is adenosine (A), or cytidine (C). In this regard, a is an integer between 1 to 4, and n is an integer of 0 or more. In an embodiment, n may be 1 or 2. In an embodiment, b may be 1, 2, 3, 4, 5, 6, 7, 8, 9, 10, 11, 12, 13, 14, 15, 16, 17, 18, 19, or 20. In an embodiment, b may be an integer within the numerical range between two of these numbers. In an embodiment, b may be an integer between 1 to 6. In an embodiment, b may be an integer between 1 to 20. In an embodiment, b may be an integer of 20 or more.

In an embodiment, the U-rich tail sequence may be represented by (UₐR)ₙ. In this regard, R is adenosine (A), or guanosine (G). In this regard, a is an integer between 1 to 4, and n is an integer of 0 or more. In an embodiment, n may be 1 or 2. In an embodiment, b may be 1, 2, 3, 4, 5, 6, 7, 8, 9, 10, 11, 12, 13, 14, 15, 16, 17, 18, 19, or 20. In an embodiment, b may be an integer within the numerical range between two of these numbers. In an embodiment, b may be an integer between 1 to 6. In an embodiment, b may be an integer between 1 to 20. In an embodiment, b may be an integer of 20 or more.

In an embodiment, the U-rich tail sequence may be represented by (UₐS)ₙ. In this regard, S is cytidine (C), or guanosine (G). In this regard, a is an integer between 1 to 4, and n is an integer of 0 or more. In an embodiment, n may be 1 or 2. In an embodiment, b may be 1, 2, 3, 4, 5, 6, 7, 8, 9, 10, 11, 12, 13, 14, 15, 16, 17, 18, 19, or 20. In this regard, b may be an integer within the numerical range between two of these numbers. In an embodiment, b may be an integer between 1 to 6. In an embodiment, b may be an integer between 1 to 20. In an embodiment, b may be an integer of 20 or more.

In an embodiment, the U-rich tail sequence may be represented by (UₐA)ₙU_{b}. In this regard, a is an integer between 1 to 4, and n is an integer of 0 or more. In an embodiment, n may be 1 or 2. In an embodiment, b may be 1, 2, 3, 4, 5, 6, 7, 8, 9, 10, 11, 12, 13, 14, 15, 16, 17, 18, 19, or 20. In this regard, b may be an integer within the numerical range between two of these numbers. In an embodiment, b may be an integer between 1 to 6. In an embodiment, b may be an integer between 1 to 20. In an embodiment, b may be an integer of 20 or more.

In an embodiment, the U-rich tail sequence may be represented by (UₐC)ₙU_{b}. In this regard, a is an integer between 1 to 4, and n is an integer of 0 or more. In an embodiment, n may be 1 or 2. In an embodiment, b may be 1, 2, 3, 4, 5, 6, 7, 8, 9, 10, 11, 12, 13, 14, 15, 16, 17, 18, 19, or 20. In this regard, b may be an integer within the numerical range between two of these numbers. In an embodiment, b may be an integer between 1 to 6. In an embodiment, b may be an integer between 1 to 20. In an embodiment, b may be an integer of 20 or more.

In an embodiment, the U-rich tail sequence may be represented by (UₐG)ₙU_{b}. In this regard, a is an integer between 1 to 4, and n is an integer of 0 or more. In an embodiment, n may be 1 or 2. In an embodiment, b may be 1, 2, 3, 4, 5, 6, 7, 8, 9, 10, 11, 12, 13, 14, 15, 16, 17, 18, 19, or 20. In this regard, b may be an integer within the numerical range between two of these numbers. In an embodiment, b may be an integer between 1 to 6. In an embodiment, b may be an integer between 1 to 20. In an embodiment, b may be an integer of 20 or more.

Example of U-Rich Tail Sequence—a Combination of Uₓ and (UₐV)ₙ

In an embodiment, the U-rich tail sequence may be in a combined form of a sequence represented by Uₓ and a sequence represented by (UₐV)ₙ. In an embodiment, the U-rich tail sequence may be represented by (U)ₙ₁-V1-(U)ₙ₂-V2-Uₓ. In this regard, V1 and V2 may each be one of adenosine (A), cytidine (C), and guanosine (G). In this regard, n1 and n2 may each be an integer between 1 to 4. In this regard, x may be an integer between 1 to 20.

U-Rich Tail Sequence—Total Length

In an embodiment, the length of the U-rich tail sequence may be 1 nt, 2 nt, 3 nt, 4 nt, 5 nt, 6 nt, 1 nt, 8 nt, 9 nt, 10 nt, 11 nt, 12 nt, 13 nt, 14 nt, 15 nt, 16 nt, 17 nt, 18 nt, 19 nt, or 20 nt. In an embodiment, the length of the U-rich tail sequence may be 20 nt or more.

U-Rich Tail Sequence—Example Sequence

In an embodiment, the U-rich tail sequence may be U, UU, UUU, UUUU, UUUUU, UUUUUU, UUUAUUU, UUUAUUUAUUU (SEQ ID NO: 11), UUUUAU, UUUUAUU, UUUUAUUU, UUUUAUUUU, UUUUAUUUUU (SEQ ID NO: 4), UUUUAUUUUUU (SEQ ID NO: 5), UUUGUUU, UUUGUUUGUUU (SEQ ID NO: 29), UUUUGU, UUUUGUU, UUUUGUUU, UUUUGUUUU, UUUUGUUUUU (SEQ ID NO: 22), or UUUUGUUUUUU (SEQ ID NO: 23).

In an embodiment, the U-rich tail sequence may be any one selected from the group consisting of SEQ ID NOS: 3 to 57.

In an embodiment, the U-rich tail sequence may be UUUUUU, UUUUAUUUUUU, or UUUUGUUUUUU.

Advantage 1 of Introducing U-Rich Tail Sequence—Increase in Gene Editing Efficiency

When the U-rich tail sequence provided in the present disclosure is introduced into the CRISPR/Cas12f1 system, the gene editing efficiency of the resulting engineered CRISPR/Cas12f1 complex may be remarkably increased. Specifically, the engineered CRISPR/Cas12f1 complex exhibits the cleavage activity on double-stranded DNA which cannot be cleaved by the wild-type CRISPR/Cas12f1 complex in eukaryotic cells, or a substantial increase in the cleavage activity on double-stranded DNA in eukaryotic cells compared to the wild-type CRISPR/Cas12f1 complex. This was experimentally proven by the present inventors and is explained in detail in the part of the Experimental Examples of the present disclosure.

Advantage 2 of Introducing U-Rich Tail Sequence—Advantages of Cas12f1 can be Used

As described above, the Cas12f1 protein has a remarkably smaller size than Cas9 and Cpf1, which are currently actively studied. Therefore, the lengths of the sequences encoding the Cas12f1 protein and guide RNA thereof are so short that the sequences can all be inserted into one AAV vector. Furthermore, even when a functional domain such as a base editor or a prime editor is added to the Cas12f1 protein, the sequences can all be included in one AAV vector. Since the AAV vector has high intracellular delivery efficiency and is approved as a gene therapy agent for humans, when the CRISPR/Cas system or a CRISPR/Cas system to which an appropriate functional domain is added is entirely included in one AAV vector, the AAV vector can be utilized for a wider gene editing technology. The total sequence of the CRISPR/Cas9 system is so long that the CRISPR/Cas9 system cannot be inserted into one AAV vector. In this aspect, the CRISPRCas12f1 system may be quite attractive as a gene editing tool. However, prior studies show that Cas12f1 proteins, particularly proteins belonging to the Cas14 family, cannot cleave double-stranded DNA or have significantly reduced cleavage efficiency. However, the present inventors have shown that the U-rich tail sequence can be introduced into the CRISPR/Cas12f1 system to significantly increase the gene editing efficiency of the CRISPR/Cas12f1 complex. In conclusion, since an engineered CRISPR/Cas12f1 complex into which the U-rich tail sequence provided in the present disclosure is introduced exhibits high nucleic acid cleavage efficiency while having the advantages of the aforementioned Cas12f1 protein, the engineered CRISPR/Cas12f1 complex can be applied to various gene editing techniques.

Engineered CRISPR RNA (crRNA) for CRISPR/Cas12f1 System

Engineered crRNA—Overview

An aspect of the present disclosure provides an engineered CRISPR RNA for the CRISPR/Cas12f1 system (hereinafter, crRNA). The engineered crRNA is a crRNA in which a U-rich tail sequence is linked to 3′-terminus of the crRNA sequence of the CRISPR/Cas12f1 system. In this regard, the case where “CRISPR RNA” or “crRNA” is described in the present disclosure without any other modification is a concept distinguished from the engineered crRNA, and basically refers to a CRISPR RNA having a configuration which is present in nature, particularly the CRISPR RNA included in the CRISPR/Cas12f1 system. The U-rich tail sequence has the same features and structure as those described in the <U-rich tail sequence> section. The crRNA includes a CRISPR RNA repeat sequence and a spacer sequence. The spacer sequence is a sequence associated with a target sequence included in a target nucleic acid to be edited by the engineered CRISPR/Cas12f1 complex. The sequence of the engineered crRNA provided in the present disclosure includes a CRISPR RNA repeat sequence, a spacer sequence, and a U-rich tail sequence.

In an embodiment, in the sequence of the engineered crRNA, the CRISPR RNA repeat sequence, the spacer sequence, and the U-rich tail sequence may be linked sequentially in the 5′-to-3′ direction.

In an embodiment, the spacer sequence may be a sequence which is complementary to the target sequence of the target nucleic acid.

In an embodiment, the U-rich tail sequence may be represented by (UₐN)ₙU_{b}. In this regard, N is one of adenosine (A), uridine (U), cytidine (C), and guanosine (G). In this regard, a is an integer between 1 to 4 inclusive, n is an integer selected from 0, 1, and 2, and b is an integer between 1 to 10 inclusive.

In an embodiment, the U-rich tail sequence may be represented by (UₐV)ₙU_{b}. In this regard, a, n, and b may be an integer, a may be 1 to 4, n may be 0 or more, and b may be 1 to 10. Additionally, an aspect of the present disclosure provides a DNA encoding the engineered crRNA. Hereinafter, the configuration of the engineered crRNA will be described in more detail.

CRISPR RNA Repeat Sequence—Definition

A CRISPR RNA repeat sequence is a sequence determined by the type of Cas12f1 protein of the CRISPR/Cas12f1 system, and refers to a sequence linked to 5′ end of the spacer sequence. The CRISPR RNA repeat sequence may vary depending on the type of Cas12f1 protein included in the CRISPR/Cas12f1 system. The CRISPR RNA repeat sequence may interact with at least some of a tracrRNA of a CRISPR/Cas12f1 system, and may also interact with a Cas12f1 protein.

CRISPR RNA Repeat Sequence—Example of Sequence

In an embodiment, the CRISPR RNA repeat sequence may be the sequence of SEQ ID NO: 58.

CRISPR RNA Repeat Sequence—Sequence can be Modified

As used herein, “CRISPR RNA repeat sequence” refers to a CRISPR RNA repeat sequence commonly found in nature, but is not limited thereto, and may refer to a CRISPR RNA repeat sequence in which part or all of the CRISPR RNA repeat sequence found in nature is modified within a range not impairing the function of the CRISPR/Cas12f1 system.

In an embodiment, the CRISPR RNA repeat sequence may be a CRISPR RNA repeat sequence in which all or part of the wild-type CRISPR RNA is modified.

In an embodiment, the CRISPR RNA repeat sequence may be a sequence which is 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50% identical or homologous to a wild-type CRISPR RNA repeat sequence. In an embodiment, the CRISPR RNA repeat sequence may be a sequence which is identical to a wild-type CRISPR RNA repeat sequence within the numerical range between two of these values. For example, the CRISPR RNA repeat sequence can be a sequence which is 90% to 100% identical to a wild-type CRISPR RNA repeat sequence. In an embodiment, the CRISPR RNA repeat sequence may be a sequence which is 60% to 80% identical to a wild-type CRISPR RNA repeat sequence.

In an embodiment, the wild-type CRISPR RNA repeat sequence may be the sequence of SEQ ID NO: 58.

Spacer Sequence—Definition

The spacer sequence is a sequence which plays a central role in allowing the CRISPR/Cas complex to exhibit target-specific gene editing activity. As used herein, a spacer sequence refers to a spacer sequence in a crRNA (or an engineered crRNA) included in the CRISPR/Cas12f1 complex, unless otherwise specified. The spacer sequence is designed to locate a target sequence included in the sequence of a target nucleic acid to be edited using the CRISPR/Cas12f1 complex. In other words, the engineered crRNA sequences provided in the present disclosure may have various spacer sequences depending on the target sequence. In this regard, the target nucleic acid may be single-stranded DNA, double-stranded DNA, or RNA.

Spacer Sequence—Relationship Between Target Nucleic Acid and Target Sequence

The spacer sequence is a sequence which is complementary to a target sequence, and is linked to a 3′-terminus side of a CRISPR RNA repeat sequence. The spacer sequence is a sequence which is homologous to a protospacer sequence adjacent to a protospacer adjacent motif (PAM) sequence recognized by the Cas12f1 protein, and has a sequence in which thymidine of the protospacer sequence is substituted with uridine. In this regard, the target sequence and the protospacer sequence are determined in a sequence adjacent to the PAM sequence included in the target nucleic acid, and accordingly, the spacer sequence is determined.

In an embodiment, the spacer sequence portion of the crRNA may complementarily bind to the target nucleic acid. In an embodiment, the spacer sequence portion of the crRNA may complementarily bind to the target sequence portion of the target nucleic acid. In an embodiment, when the target nucleic acid is double-stranded DNA, the spacer sequence may be a sequence which is complementary to a target sequence included on a target strand of the double-stranded DNA. In an embodiment, when the target nucleic acid is double-stranded DNA, the spacer sequence may be a sequence which is homologous to a protospacer sequence included on a non-target strand of the double-stranded DNA. Specifically, the spacer sequence has the same base sequence as the protospacer sequence, but may have a sequence in which each of the thymidines (T) included in the base sequence is substituted with uridine (U). In an embodiment, the spacer sequence may be an RNA sequence corresponding to the DNA sequence of the protospacer.

Spacer Sequence—Length of Sequence

In an embodiment, the length of the spacer sequence may be 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, or 40 nt. In an embodiment, the spacer sequence may have a length within the numerical range between two of these numbers. For example, the length of the spacer sequence may be 17 nt to 23 nt. In an embodiment, the length of the spacer sequence may be 17 nt to 30 nt.

U-Rich Tail Sequence

The engineered crRNA is characterized in that the U-rich tail sequence is linked to the 3′-terminus portion of the spacer sequence included in the crRNA sequence. The U-rich tail sequence has the same configuration and features as those described in the <U-rich tail sequence> section.

DNA Encoding Engineered crRNA

An aspect of the present disclosure provides a DNA encoding the engineered crRNA. In an embodiment, the DNA sequence may have a sequence in which all uridines are substituted with thymidine in the sequence of the engineered crRNA.

Use of Engineered crRNA

The engineered crRNA may constitute an engineered guide RNA for the CRISPR/Cas12f1 system along with a tracrRNA for Cas12f1. The engineered guide RNA including the engineered crRNA may bind to the Cas12f1 protein to form a CRISPR/Cas12f1 complex.

Engineered Guide RNA for CRISPR/Cas12f1 System

Engineered Guide RNA—Overview

An aspect of the present disclosure provides an engineered guide RNA for the CRISPR/Cas12f1 system. The sequence of the engineered guide RNA includes a tracrRNA sequence, a crRNA sequence, and a U-rich tail sequence. In this regard, the U-rich tail sequence is linked to 3′-terminus of the crRNA sequence. In other words, the sequence of the engineered guide RNA includes a tracrRNA sequence and an engineered crRNA sequence. The crRNA includes a CRISPR RNA repeat sequence and a spacer sequence. In this regard, the CRISPR RNA repeat sequence and the spacer sequence have the same features and structure as each component described in the <Engineered CRISPR RNA for CRISPR/Cas12f1 system> section. The U-rich tail has the same features and structure as those described in the <U-rich tail sequence> section.

In an embodiment, the U-rich tail sequence may be represented by (UₐN)ₙU_{b}. In this regard, N is one of adenosine (A), uridine (U), cytidine (C), and guanosine (G). In this regard, a is an integer between 1 to 4, n is an integer selected from 0, 1, and 2, and b is an integer between 1 to 10 inclusive.

In an embodiment, the U-rich tail sequence may be represented by (UₐV)ₙU_{b}, and in this regard, a, n, and b are an integer, a may be between 1 to 4 inclusive, n may be 0 or more, and b may be between 1 to 10 inclusive.

At least a part of the tracrRNA included in the engineered guide RNA, and at least a part of the crRNA, may have a sequence which is complementary to each other to form a double-stranded RNA. Specifically, at least a part of the tracrRNA, and at least a part of the CRISPR RNA repeat sequence included in the crRNA sequence may be complementary to each other to form a double-stranded RNA. The engineered guide RNA may bind to the Cas12f1 protein to form a CRISPR/Cas12f1 complex, and recognize a target sequence which is complementary to a spacer sequence included in the crRNA sequence to allow the CRISRP/Cas12f1 complex to edit a target nucleic acid including the target sequence.

tracrRNA—Definition

A tracrRNA, along with crRNA, is an important component which constitutes the guide RNA of the CRISPR/Cas system. As used herein, the tracrRNA refers to a tracrRNA which constitutes the guide RNA of the CRISPR/Cas12f1 system, unless otherwise described. At least a part of the tracrRNA may complementarily bind to at least a part of crRNA to form a double-strand. More specifically, a partial sequence of the tracrRNA has a sequence which is complementary to all or part of the CRISPR RNA repeat sequence included in the crRNA.

tracrRNA—Complementarity of tracrRNA and CRISPR RNA Repeat Sequence

In an embodiment, the sequence of the tracrRNA may include a sequence which is 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to the CRISPR RNA repeat sequence. In an embodiment, the sequence of the tracrRNA may include a sequence which is complementary to the CRISPR RNA repeat sequence within the numerical range between two of these numbers. For example, the sequence of the tracrRNA may include a sequence which is 60% to 90% complementary to the CRISPR RNA repeat sequence. In an embodiment, the sequence of the tracrRNA may include a sequence which is 70% to 100% complementary to the CRISPR RNA repeat sequence.

tracrRNA—Number of Mismatches in tracrRNA and CRISPR RNA Repeat Sequence

In an embodiment, the sequence of the tracrRNA may include a complementary sequence with 0, 1, 2, 3, 4, 5, 6, 7, 8, 9, 10, 11, 12, 13, 14, 15, 16, 17, 18, 19, or 20 mismatches with the CRISPR RNA repeat sequence. In an embodiment, the complementary sequence of the tracrRNA may have mismatches with the CRISPR RNA repeat sequence within the numerical range selected in the immediately preceding sentence. For example, the tracrRNA may include a complementary sequence with 0 to 8 mismatches with the CRISPR RNA repeat sequence. In an embodiment, the tracrRNA sequence may include a complementary sequence with 8 to 12 mismatches with the CRISPR RNA repeat sequence.

tracrRNA—Example of Sequence

In an embodiment, the tracrRNA sequence may be the sequence of SEQ ID NO: 60.

tracrRNA—Sequence can be Modified

As used herein, “tracrRNA” refers to a tracrRNA commonly found in nature, but is not limited thereto, and may refer to a tracrRNA in which part or all of the tracrRNA sequence found in nature is modified within a range not impairing the function of the CRISRP/Cas12f1 system.

In an embodiment, the tracrRNA sequence may be a tracrRNA sequence in which all or part of a wild type tracrRNA sequence is modified.

In an embodiment, the tracrRNA repeat sequence may be a sequence which is 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50% identical or homologous to a wild-type tracrRNA repeat sequence. In an embodiment, the tracrRNA sequence may be a sequence which is identical to a wild-type tracrRNA sequence within the numerical range between two of these numbers. For example, the tracrRNA sequence may be a sequence which is 90% to 100% identical to a wild-type tracrRNA sequence. In an embodiment, the tracrRNA sequence may be a sequence which is 60% to 80% identical to a wild-type tracrRNA sequence.

In an embodiment, the wild-type tracrRNA sequence may be the sequence of SEQ ID NO: 60.

Dual Guide RNA—Definition and Structure

The engineered guide RNA provided in the present disclosure may be a dual guide RNA. This means that the tracrRNA and the crRNA form separate RNA molecules. A part of the tracrRNA and a part of the crRNA complementarily bind to each other to form a double strand. Specifically, in the dual guide RNA, a part including a 3′-terminus of the tracrRNA and a part including the CRISPR RNA repeat sequence of the crRNA may form a double strand.

In an embodiment, the engineered guide RNA may be a dual guide RNA. In an embodiment, when the engineered guide RNA is a dual guide RNA, the tracrRNA and the crRNA are separate RNA molecules.

Dual Guide RNA—Example of Sequence

In an embodiment, the tracrRNA may be the sequence of SEQ ID NO: 60. In an embodiment, the CRISPR RNA repeat sequence included in the crRNA may be the sequence of SEQ ID NO: 58. In an embodiment, at least a part of a trancrRNA having the sequence of SEQ ID NO: 60 and the CRISPR RNA repeat sequence having the sequence of SEQ ID NO: 58 may complementarily bind to each other to form a double strand.

Single Guide RNA—Definition

In order to facilitate the formation of a guide RNA, the design of the tracrRNA and the crRNA into one molecule is referred to as a single guide RNA. The engineered guide RNA provided in the present disclosure may be a single guide RNA. When the engineered guide RNA is a single guide RNA, the single guide RNA is one molecule of RNA including all of the tracrRNA, the crRNA, and the U-rich tail. In an embodiment, when the engineered guide RNA is a single guide RNA, the sequence of the tracrRNA and the sequence of the crRNA may both be included in the sequence of one molecule of RNA.

Single Guide RNA—Example of Linker and Sequence

In an embodiment, when the engineered guide RNA is a single guide RNA, the sequence of the engineered guide RNA further comprises a linker sequence, and the tracrRNA sequence and the crRNA sequence may be linked via the linker sequence. In an embodiment, the linker sequence may be 5′-gaaa-3′.

Single Guide RNA—Structure

In the sequence of the single guide RNA, a tracrRNA sequence, a linker sequence, a crRNA sequence, and a U-rich tail sequence are linked sequentially in the 5′-to-3′ direction. A part of the tracrRNA sequence and all or part of the CRISPR RNA repeat sequence included in the crRNA sequence have a sequence which is complementary to each other. Therefore, a part of the tracrRNA, and the CRISPR RNA repeat sequence part in the crRNA complementarily bind to each other to form a double-stranded RNA.

Single Guide RNA—Example of Sequence

The single guide RNA may have a sequence selected from the group consisting of SEQ ID NOS: 75 to 125 and 261 to 280.

Scaffold Sequence

When the sequences of the engineered guide RNA provided in the present disclosure are functionally divided, the sequences may be divided into 1) a sequence portion which interacts with a Cas12f1 protein to form the CRISPR/Cas12f1 complex, 2) a sequence portion which allows the CRISPR/Cas12f1 complex to find a target nucleic acid, and 3) a U-rich tail sequence portion. In this regard, the sequence portion which interacts with the Cas12f1 protein to form the CRISPR/Cas12f1 complex may be said to be a scaffold sequence. In this regard, the scaffold sequence collectively refers to a portion which interacts with the Cas12f1 protein, and is not limited to the RNA sequence of one molecule. Specifically, the scaffold sequence may include sequences of two or more molecules of RNA.

In an embodiment, when the engineered guide RNA is a dual guide RNA, the scaffold sequence may include a tracrRNA sequence and a CRISPR RNA repeat sequence included in a crRNA among the engineered guide RNA sequences. In an embodiment, the tracrRNA sequence may be a tracrRNA sequence in which all or part of the naturally occurring tracrRNA sequence is modified. In an embodiment, the CRISPR RNA repeat sequence may be a CRISPR RNA sequence in which all or part of a CRISPR RNA repeat sequence found in nature is modified.

In an embodiment, when the engineered guide RNA is a single guide RNA, the scaffold sequence may include the tracrRNA sequence, the linker sequence, and a CRISPR RNA repeat sequence included in the crRNA sequence. In an embodiment, the tracrRNA sequence may be a tracrRNA sequence in which all or part of the naturally occurring tracrRNA sequence is modified. In an embodiment, the CRISPR RNA repeat sequence may be a CRISPR RNA sequence in which all or part of a CRISPR RNA repeat sequence found in nature is modified.

In an embodiment, the scaffold sequence may be a sequence selected from the group consisting of SEQ ID NOS: 253 and 254. In an embodiment, the scaffold sequence may be a sequence which is 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50% identical or homologous to the sequence selected from the group consisting of SEQ ID NOS: 253 and 254. In an embodiment, the scaffold sequence may be a sequence which is identical to the sequence selected from the group consisting of SEQ ID NOS: 253 and 254 within the numerical range between two of these numbers. For example, the scaffold sequence may be a sequence which is 90% to 100% identical to the scaffold sequence selected from the group consisting of SEQ ID NOS: 253 and 254. In an embodiment, the scaffold sequence may be a sequence which is 60% to 80% identical to the scaffold sequence selected from the group consisting of SEQ ID NOS: 253 and 254.

Engineered CRISPR/Cas12f1 Complex

CRISPR/Cas12f1 Complex—Overview

An aspect of the present disclosure provides an engineered CRISPR/Cas12f1 complex. The engineered CRISPR/Cas12f1 complex comprises a Cas12f1 protein and an engineered guide RNA. In an embodiment, the Cas12f1 protein may be derived from the Cas14 family. The engineered guide RNA sequence includes a tracrRNA sequence, a crRNA sequence, and a U-rich tail sequence. In other words, the engineered guide RNA sequence includes a scaffold sequence, a spacer sequence, and a U-rich tail sequence. The U-rich tail sequence may be linked to 3′-terminus of the crRNA sequence. The U-rich tail has the same features and structure as those described in the <U-rich tail sequence> section.

In an embodiment, the U-rich tail sequence may be represented by (UₐN)ₙU_{b}. In this regard, N is one of adenosine (A), uridine (U), cytidine (C), and guanosine (G). In this regard, a is an integer between 1 to 4, n is an integer selected from 0, 1, and 2, and b is an integer between 1 to 10 inclusive.

In an embodiment, the U-rich tail sequence may be represented by (UₐV)ₙU_{b}, and in this regard, a, n, and b are an integer, a may be between 1 to 4, n may be 0 or more, and b may be between 1 to 10.

The scaffold sequence may interact with the Cas12f1 protein, and plays an important role when the engineered guide RNA and the Cas12f1 protein form a complex.

Cas12f1 Protein—Overview

The engineered CRISPR/Cas12f1 complex may include a Cas12f1 protein. Basically, the Cas12f1 protein may be a wild-type Cas12f1 protein which is present in nature. The sequence encoding the Cas12f1 protein may be a human codon-optimized Cas12f1 sequence for the wild-type Cas12f1 protein. Further, the Cas12f1 protein may have the same function as a wild-type Cas12f1 protein which is present in nature. However, unless otherwise specified, as used herein, the “Cas12f1 protein” may refer to not only a wild-type or codon-optimized Cas12f1 protein but also a modified Cas12f1 protein or Cas12f1 fusion protein. In addition, the Cas12f1 protein may collectively refer to not only a Cas12f1 protein having the same function as a wild-type Cas12f1 protein, but also a Cas12f1 protein in which all or part of the function is modified, a Cas12f1 protein in which all or part of the function is lost, and/or a Cas12f1 protein to which an additional function is added. The meaning of the Cas12f1 protein may be appropriately interpreted depending on the context, and is interpreted in the broadest sense unless there are special cases. Hereinafter, the configuration or function of the Cas12f1 protein will be described in detail.

Cas12f1 Protein—Wild-Type Cas12f1 Protein

The engineered CRISPR/Cas12f1 complex may include a Cas12f1 protein. In an embodiment, the Cas12f1 protein may be a wild-type Cas12f1 protein. In an embodiment, the Cas12f1 protein may be derived from the Cas14 family (Harrington et al., Programmed DNA destruction by miniature CRISPR-Cas14 enzymes, Science 362, 839-842 (2018)). In an embodiment, the Cas12f1 protein may be a Cas14a protein derived from uncultured archaeons (Harrington et al., Programmed DNA destruction by miniature CRISPR-Cas14 enzymes, Science 362, 839-842 (2018)). In an embodiment, the Cas12f1 protein may be a Cas14a1 protein.

Cas12f1 Protein—Modified Cas12f1 Protein

The engineered CRISPR/Cas12f1 complex may include a modified Cas12f1 protein. The modified Cas12f1 refers to a Cas12f1 in which at least some sequences of wild-type or codon-optimized Cas12f1 protein sequences are modified. The Cas12f1 protein modification may consist of individual amino acid units, and may consist of functional domain units of the protein. In an embodiment, the modification of the protein may be a modification in which one or more amino acids, peptides, polypeptides, proteins, and/or domains are individually substituted for, deleted from, and/or added to a wild-type or codon-optimized Cas12f1 protein sequence. In an embodiment, the Cas12f1 protein may be a Cas12f1 protein which one or more amino acids, peptides, and/or polypeptides in a RuvC domain included in a wild-type Cas12f1 protein are substituted for, deleted from, and added to.

Cas12f1 Protein—Cas12f1 Fusion Protein

The engineered CRISPR/Cas12f1 complex provided in the present disclosure may include a Cas12f1 fusion protein. In this regard, the Cas12f1 fusion protein refers to a protein in which a wild-type or modified Cas12f1 protein is fused with an additional amino acid, peptide, polypeptide, protein, or domain.

In an embodiment, a Cas12f1 protein may be a fusion of a wild-type Cas12f1 protein with a base editor and/or reverse transcriptase. In an embodiment, the base editor may be adenosine deaminase, and/or cytosine deaminase. In an embodiment, the reverse transcriptase may be a Moloney Murine Leukemia Virus (M-MLV) reverse transcriptase, or a variant thereof. In this regard, a Cas12f1 protein fused with the reverse transcriptase may function as a prime editor.

In an embodiment, a Cas12f1 protein may be a fusion of a wild-type Cas12f1 protein with various enzymes capable of participating in the intracellular gene expression process. In this regard, the Cas12f1 protein fused with the enzymes may bring about various quantitative and qualitative changes in gene expression in the cell. In an embodiment, the enzyme may be DNMT, TET, KRAB, DHAC, LSD, and/or p300.

Cas12f1 Protein—Change in Function

The Cas12f1 protein included in the engineered CRISPR/Cas12f1 complex provided in the present disclosure may have similar functions to the wild-type Cas12f1 protein. The Cas12f1 protein included in the engineered CRISPR/Cas12f1 complex provided in the present disclosure may be a Cas12f1 protein with a changed function when compared to the wild-type Cas12f1 protein. Specifically, the change may be a modification of all or part of the function, a loss of all or part of the function, or an addition of additional functions. In an embodiment, the Cas12f1 protein is not particularly limited as long as it has a modification applicable to the Cas protein of the CRISPR/Cas system by those skilled in the art. In this regard, the change may be made by a known technique. In an embodiment, the Cas12f1 protein may be modified to cleave only one strand of the double strands of a target nucleic acid. Furthermore, the Cas12f1 protein may be changed such that only one strand of the double strands of the target nucleic acid can be cleaved and base editing or prime editing can be performed on the uncleaved strand. In an embodiment, the Cas12f1 protein may be changed such that both of the double strands of the target nucleic acid cannot be cleaved. Furthermore, the Cas12f1 protein may be changed such that both of the double strands of the target nucleic acid cannot be cleaved, and base editing, prime editing, or gene expression regulation function can be performed on the target nucleic acid.

Cas12f1 Protein—Example of Other Modifications

In an embodiment, the Cas12f1 protein may include a nuclear localization sequence (NLS), or a nuclear export sequence (NES). Specifically, the NLS may be any one of those exemplified in the NLS section in the <Definition of terms>, but is not limited thereto. In an embodiment, the Cas12f1 protein may include a tag. Specifically, the tag may be any one of those exemplified in the tag section in the <Definition of terms>, but is not limited thereto.

Cas12f1 Protein—PAM Sequence

Two conditions are required for the CRISPR/Cas12f1 complex to cleave a target gene or target nucleic acid. First, there must be a base sequence having a certain length, which the Cas12f1 protein can recognize in the target gene or the target nucleic acid. Second, there must be a sequence capable of complementarily binding to a spacer sequence included in a guide RNA around the base sequence having a certain length. When the two conditions are satisfied to allow 1) the Cas12f1 protein recognize the base sequence having a certain length and 2) the spacer sequence portion complementarily bind to the sequence portion around the base sequence having a certain length, the target gene or the target nucleic acid is cleaved. In this regard, the base sequence having a certain length recognized by the Cas12f1 protein is called a protospacer adjacent motif (PAM) sequence. The PAM sequence is a unique sequence determined according to the Cas12f1 protein, and is accompanied by a constraint that the target sequence must be determined in the sequences adjacent to the PAM sequence, when the target sequence of the CRISPR Cas12f1 complex is determined.

Cas12f1 Protein—Example of PAM Sequence

In an embodiment, the PAM sequence of the Cas12f1 protein may be a T-rich sequence. In an embodiment, the PAM sequence of the Cas12f1 protein may be TTTN in the 5′-to-3′ direction. In this regard, N is one of deoxythymidine (T), deoxyadenosine (A), deoxycytidine (C), or deoxyguanosine (G). In an embodiment, the PAM sequence of the Cas12f1 protein may be TTTA, TTTT, TTTC, or TTTG in the 5′-to-3′ direction. In an embodiment, the PAM sequence of the Cas12f1 protein may be TTTA or TTTG in the 5′-to-3′ direction. In an embodiment, the PAM sequence of the Cas12f1 protein may be different from a PAM sequence of a wild-type Cas12f1 protein.

Cas12f1 Protein—Example Sequence

In an embodiment, the Cas12f1 protein may have an amino acid sequence selected from the group consisting of SEQ ID NOS: 281, 317, 319, 321, and 323.

In an embodiment, the DNA sequence encoding the Cas12f1 protein may be a human codon-optimized sequence.

In an embodiment, the DNA sequence encoding the Cas12f1 protein may be a DNA sequence selected from the group consisting of SEQ ID NOS: 1, 2, 316, 318, 320, and 322.

Engineered Guide RNA

An engineered guide RNA constituting the CRISPR/Cas12f1 complex provided in the present disclosure have the same features and structure as those described in the <Engineered guide RNA for CRISPR/Cas12f1 system> section.

CRISPR/Cas12f1 Complex—Example of Configuration

In an embodiment, the Cas12f1 protein may have an amino acid sequence of SEQ ID NO: 281, the engineered guide RNA may have an amino acid sequence selected from the group consisting of SEQ ID NOS: 75 to 125, and the Cas12f1 protein and the engineered guide RNA may bind to each other to form a CRISPR/Cas12f1 complex.

In an embodiment, the Cas12f1 protein may have an amino acid sequence selected from the group consisting of SEQ ID NOS: 317, 319, 321, and 323, the engineered guide RNA may have a sequence selected from the group consisting of SEQ ID NOS: 75 to 125 and 261 to 280, and the CRISPR/Cas12f1 complex formed by binding the Cas12f1 protein and the engineered guide RNA may have a base editing function.

Vector for Expressing CRISPR/Cas12f1 System

Overview

An aspect of the present disclosure provides a vector for expressing the components of a CRISPR/Cas12f1 system. The vector is constructed to express the Cas12f1 protein and/or the engineered guide RNA. The sequence of the vector may include a nucleic acid sequence encoding one of the components of the CRISPR/Cas12f1 system, or may include a nucleic acid sequence encoding two or more components. The sequence of the vector includes a nucleic acid sequence encoding the Cas12f1 protein and/or a nucleic acid sequence encoding the engineered guide RNA. The sequence of the vector includes one or more promoter sequences. The promoter is operably linked to a nucleic acid sequence encoding the Cas12f1 protein and/or a nucleic acid sequence encoding the engineered guide RNA to promote transcription of the nucleic acid sequence in the cell. The Cas12f1 protein has the same features and configuration as those of the Cas12f1 protein, the modified Cas12f1 protein, or the Cas12f1 fusion protein described in the <Engineered CRISPR/Cas12f1 complex> section. The engineered guide RNA has the same features and configuration as those of the engineered guide RNA described in the <Engineered guide RNA for CRISPR/Cas12f1 system> section.

The sequence of the vector may include a nucleic acid sequence encoding the Cas12f1 protein and a nucleic acid sequence encoding the engineered guide RNA. In an embodiment, the sequence of the vector may include a first sequence including a nucleic acid sequence encoding the Cas12f1 protein and a second sequence including a nucleic acid sequence encoding the engineered guide RNA. The sequence of the vector includes a promoter sequence for expressing the nucleic acid sequence encoding the Cas12f1 protein in the cell, and a promoter sequence for expressing the nucleic acid sequence encoding the engineered guide RNA in the cell, and the promoters are operably linked to each expression target. In an embodiment, the sequence of the vector may include a first promoter sequence operably linked to the first sequence, and a second promoter sequence operably linked to the second sequence.

The sequence of the vector may include a nucleic acid sequence encoding the Cas12f1 protein and a nucleic acid sequence encoding two or more different engineered guide RNAs. In an embodiment, the sequence of the vector may include a first sequence including a nucleic acid sequence encoding the Cas12f1 protein, a second sequence including a nucleic acid sequence encoding the engineered guide RNA, and a third sequence including a nucleic acid sequence encoding the second engineered guide RNA. Furthermore, the sequence of the vector may include a first promoter sequence operably linked to the first sequence, a second promoter sequence operably linked to the second sequence, and a third promoter sequence operably linked to the third sequence.

Expression Target—Cas12f1 Protein

The vector may be constructed to express a Cas12f1 protein. In this regard, the Cas12f1 protein has the same configuration and features as those described in the <Engineered CRISPR/Cas12f1 complex> section. In an embodiment, the vector may be constructed to express a wild-type Cas12f1 protein. In this regard, the wild-type Cas12f1 protein may be Cas14a1. In an embodiment, the vector may be constructed to express a Cas12f1 protein which has been changed to cleave only one strand of the double strands of the target nucleic acid. Furthermore, the changed Cas12f1 protein may be changed such that only one strand of the double strands of the target nucleic acid can be cleaved and base editing or prime editing can be performed on the uncleaved strand. In an embodiment, the Cas12f1 protein may have been changed such that both of the double strands of the target nucleic acid cannot be cleaved. Furthermore, the Cas12f1 protein may be changed such that both of the double strands of the target nucleic acid cannot be cleaved, and base editing, prime editing, or gene expression regulation function can be performed on the target nucleic acid.

Expression Target—Engineered Guide RNA

The vector may be constructed to express an engineered guide RNA. The engineered guide RNA has the same features and configuration as those of the engineered guide RNA described in the <Engineered guide RNA for CRISPR/Cas12f1 system> section. In an embodiment, the vector may be constructed to express an engineered guide RNA. In an embodiment, the engineered guide RNA sequence may include a scaffold sequence, a spacer sequence, and a U-rich tail sequence. In an embodiment, the engineered guide RNA sequence may include a tracrRNA sequence, a crRNA sequence, and a U-rich tail sequence. In an embodiment, the U-rich tail sequence may be represented by (UₐN)ₙU_{b}. In this regard, N is one of adenosine (A), uridine (U), cytidine (C), and guanosine (G). In this regard, a is an integer between 1 to 4 inclusive, n is an integer selected from 0, 1, and 2, and b is an integer between 1 to 10 inclusive. In an embodiment, the U-rich tail sequence may be represented by (UₐV)ₙU_{b}. In this regard, a, n, and b may be an integer, a may be 1 to 4, n may be 0 or more, and b may be 1 to 10.

The vector may be constructed to express two or more engineered guide RNAs. In an embodiment, the vector may be constructed to express a first engineered guide RNA and a second engineered guide RNA. In an embodiment, the first engineered guide RNA sequence may include a first scaffold sequence, a first spacer sequence, and a first U-rich tail sequence, and the second engineered guide RNA sequence may include a second scaffold sequence, a second spacer sequence, and a second U-rich tail sequence.

Expression Target—Additional Component

The vector may be constructed to express an additional component such as NLS and a tag protein in addition to the above-described expression targets. In an embodiment, the additional component may be expressed independently from the Cas12f1, the modified Cas12f1, and/or the engineered guide RNA. In an embodiment, the additional component may be expressed while being linked to the Cas12f1, the modified Cas12f1, and/or the engineered guide RNA. In this regard, the additional component may be a component which is generally expressed when the CRISPR/Cas system is intended to be expressed, and a known technique may be referenced. The additional component may be one or more of the components described in the <Related Art—design of CRISPR/Cas system expression vector> section.

Vector Component—Cas12f1 Protein Expression Sequence

The vector sequence may include a nucleic acid sequence encoding the Cas12f1 protein. In this regard, the Cas12f1 protein has the same configuration and features as those described in the <Engineered CRISPR/Cas12f1 complex> section. In an embodiment, the sequence of the vector may include a sequence encoding a wild-type Cas12f1 protein. In this regard, the wild-type Cas12f1 protein may be Cas14a1. In an embodiment, the sequence of the vector may include a human codon-optimized nucleic acid sequence encoding a Cas12f1 protein. In this regard, the human codon-optimized nucleic acid sequence encoding the Cas12f1 protein may be a human codon-optimized nucleic acid sequence encoding a Cas14a1 protein. In an embodiment, the sequence of the vector may be a sequence encoding a Cas12f1 protein or a Cas12f1 fusion protein. In an embodiment, the sequence of the vector may include a sequence encoding a Cas12f1 protein changed such that only one strand of the double strands of the target nucleic acid can be cleaved and base editing or prime editing can be performed on the uncleaved strand. In an embodiment, the Cas12f1 protein may include a sequence encoding a Cas12f1 protein changed such that both of the double strands of the target nucleic acid cannot be cleaved, and base editing, prime editing, or gene expression regulation function can be performed on the target nucleic acid.

Vector Component—Engineered Guide RNA Expression Sequence

In an embodiment, the sequence of the vector may include a sequence encoding an engineered guide RNA. In an embodiment, the sequence of the engineered guide RNA may include a scaffold sequence, a spacer sequence, and a U-rich tail sequence. In an embodiment, the sequence of the engineered guide RNA sequence may include a tracrRNA sequence, a crRNA sequence, and a U-rich tail sequence. In an embodiment, the U-rich tail sequence may be represented by (UₐN)ₙU_{b}. In this regard, N is one of adenosine (A), uridine (U), cytidine (C), and guanosine (G). In this regard, a is an integer between 1 to 4 inclusive, n is an integer selected from 0, 1, and 2, and b is an integer between 1 to 10 inclusive. In an embodiment, the U-rich tail sequence may be represented by (UₐV)ₙU_{b}. In this regard, a, n, and b may be an integer, a may be 1 to 4, n may be 0 or more, and b may be 1 to 10.

In an embodiment, the sequence of the vector may include a sequence encoding two or more different engineered guide RNAs. In an embodiment, the sequence of the vector may include a sequence encoding a first engineered guide RNA, and a sequence encoding a second engineered guide RNA. In this regard, a spacer sequence included in the first engineered guide RNA sequence may be different from a spacer sequence included in the second engineered guide RNA sequence. Furthermore, a U-rich tail sequence included in the first engineered guide RNA sequence may be different from a U-rich tail sequence included in the second engineered guide RNA sequence. In an embodiment, the first engineered guide RNA sequence may include a first scaffold sequence, a first spacer sequence, and a first U-rich tail sequence, and the second engineered guide RNA sequence may include a second scaffold sequence, a second spacer sequence, and a second U-rich tail sequence.

Vector Component—Promoter Sequence

The vector sequence includes a promoter sequence operably linked to a sequence encoding each component. Specifically, the promoter sequence may be one of the promoters disclosed in the promoter portion in the <Related Art—design of CRISPR/Cas system expression vector> section, but is not limited thereto.

In an embodiment, the vector sequence may include a sequence encoding a Cas12f1 protein, and a promoter sequence. In this regard, the promoter sequence is operably linked to a sequence encoding the Cas12f1 protein. In an embodiment, the vector sequence may include a sequence encoding an engineered guide RNA, and a promoter sequence. In this regard, the promoter sequence is operably linked to a sequence encoding the engineered guide RNA. In an embodiment, the vector sequence may include a sequence encoding a Cas12f1 protein, a sequence encoding an engineered guide RNA, and a promoter sequence. In this regard, the promoter sequence is operably linked to a sequence encoding the Cas12f1 protein and a sequence encoding the engineered guide RNA, and a transcription factor activated by the promoter sequence expresses the Cas12f1 protein and the engineered guide RNA.

Vector Configuration—Two or More Promoter Sequences can be Included

In an embodiment, the vector sequence may include a first sequence encoding a Cas12f1 protein, a first promoter sequence, a second sequence encoding an engineered guide RNA, and a second promoter sequence. In this regard, the first promoter sequence is operably linked to the first sequence, the second promoter sequence is operably linked to the second sequence, transcription of the first sequence is induced by the first promoter sequence, and transcription of the second sequence is induced by the second promoter sequence. In this regard, the first promoter and the second promoter may be the same type of promoter. In this regard, the first promoter and the second promoter may be different types of promoters.

In an embodiment, the vector sequence may include a first sequence encoding a Cas12f1 protein, a first promoter sequence, a second sequence encoding a first engineered guide RNA, a third sequence including a sequence that encodes a second engineered guide RNA, and a third promoter sequence. In this regard, the first promoter sequence is operably linked to the first sequence, the second promoter sequence is operably linked to the second sequence, the third promoter sequence is operably linked to the third sequence, transcription of the first sequence is induced by the first promoter sequence, transcription of the second sequence is induced by the second promoter sequence, and transcription of the third sequence is induced by the third promoter sequence. In this regard, the second promoter and the third promoter may be the same type of promoter. Specifically, the second promoter sequence and the third promoter sequence may be a U6 promoter sequence, but are not limited thereto. In this regard, the second promoter and the third promoter may be different types of promoters. Specifically, the second promoter sequence and the third promoter sequence may be a U6 promoter sequence and a promoter sequence, respectively, but are not limited thereto.

Vector Component—Termination Signal

The vector may include a termination signal operably linked to the promoter sequence. In this regard, the termination signal may be one of the termination signals disclosed in the termination signal portion in the <Related Art—design of CRISPR/Cas system expression vector> section, but is not limited thereto. The termination signal may vary depending on the type of promoter sequence. In an embodiment, when the vector sequence includes a U6 promoter sequence, a continuous thymidine sequence operably linked to the U6 promoter sequence may act as a termination signal. In an embodiment, the thymidine sequence may be a sequence in which five or more thymidines are continuously linked. In an embodiment, when the vector sequence includes a H1 promoter sequence, a continuous thymidine sequence operably linked to the H1 promoter sequence may act as a termination signal. In an embodiment, the thymidine sequence may be a sequence in which five or more thymidines are continuously linked.

Vector Component—Relationship Between Sequence Encoding U-Rich Tail and Termination Signal

The sequence of the engineered guide RNAs provided in the present disclosure includes a U-rich tail sequence at 3′-terminus thereof. Therefore, the sequence encoding the engineered guide RNA will include a T-rich sequence corresponding to the U-rich tail sequence at 3′-terminus thereof. As described above, since some promoter sequences recognize a continuous thymidine sequence, for example, a sequence in which five or more thymidines are continuously linked as a termination signal, the T-rich sequence may be recognized as a termination signal in some cases. In other words, when the vector sequence according to the present disclosure includes a sequence encoding an engineered guide RNA, the sequence encoding the U-rich tail sequence included in the engineered guide RNA sequence may be used as a termination signal. In an embodiment, when the vector sequence a U6 or H1 promoter sequence, and includes a sequence encoding an engineered guide RNA operably linked to the U6 or H1 promoter sequence, a sequence portion encoding a U-rich tail sequence included in the engineered guide RNA sequence may be recognized as a termination signal. In this regard, the U-rich tail sequence includes a sequence in which five or more uridines are continuously linked.

Vector Component—Other Components

The vector sequence may include a component required depending on the purpose, in addition to the configuration. In an embodiment, the vector sequence may include a regulatory/control component sequence and/or an additional component sequence. In an embodiment, the additional component may be added for the purpose of distinguishing transfected cells from non-transfected cells. In this regard, the regulatory/control component sequence and the additional component may be one of those disclosed in the <Related Art—design of CRISPR/Cas system expression vector>, but is not limited thereto.

Type of Vector—Viral Vector

The vector may be a viral vector. In an embodiment, the viral vector may be one or more selected from the group consisting of a retrovirus, a lentivirus, an adenovirus, an adeno-associated virus (AAV), a vaccinia virus, a poxvirus and a herpes simplex virus. In an embodiment, the viral vector may be adeno-associated virus (AAV).

Type of Vector—Non-Viral Vector

The vector may be a non-viral vector. In an embodiment, the non-viral vector may be one or more selected from the group consisting of a plasmid, a phage, naked DNA, a DNA complex, and mRNA. In an embodiment, the plasmid may be selected from the group consisting of the pcDNA series, pSC101, pGV1106, pACYC177, ColE1, pKT230, pME290, pBR322, pUC8/9, pUC6, pBD9, pHC79, pIJ61, pLAFR1, pHV14, the pGEX series, the pET series, and pUC19. In an embodiment, the phage may be selected from the group consisting of λgt4λB, λ-Charon, λΔz1, and M13. In an embodiment, the vector may be a PCR amplicon.

Vector Form—Circular or Linear Vector

The vector may be in a circular or linear form. When the vector is a linear vector, RNA transcription is terminated at a 3′-terminus thereof, even though the linear vector sequence does not include a termination signal separately. In comparison, when the vector is a circular vector, RNA transcription is not terminated unless the circular vector sequence includes a termination signal separately. Therefore, when a circular vector is used as the vector, a termination signal corresponding to a transcription factor related to each promoter sequence must be included in order to express an intended target. In an embodiment, the vector may be a linear vector. In an embodiment, the vector may be a linear amplicon. In an embodiment, the vector may be a linear vector including a sequence selected from the group consisting of SEQ ID NOS: 1, 2, and 127 to 177. In an embodiment, the vector may be a circular vector. In an embodiment, the vector may be a circular vector including a sequence selected from the group consisting of SEQ ID NOS: 1, 2, and 127 to 177.

Vector—Example of Sequence

In an embodiment, the vector sequence may include one or more sequences selected from the group consisting of SEQ ID NOS: 1, 2, and 127 to 177. In an embodiment, the vector sequence may include one or more sequences selected from the group consisting of SEQ ID NOS: 1, 2, 316, 318, 320, and 322.

Chemical Modification of Nucleic Acid

An aspect of the present disclosure provides a component including or consisting of an engineered crRNA or a nucleic acid encoding the engineered crRNA, an engineered guide RNA or a nucleic acid encoding the engineered guide RNA, and/or a nucleic acid such as a vector for expressing a component of a CRISPR/Cas12f1 system. In this regard, the “nucleic acid” used herein may be DNA or RNA present in nature, and may be a modified nucleic acid in which part or all of the nucleic acid is chemically modified. In an embodiment, the nucleic acid may be DNA and/or RNA present in nature. In an embodiment, the nucleic acid may be a nucleic acid in which one or more nucleotides are chemically modified. In this regard, the chemical modification includes all nucleic acid modifications known in the art. Specifically, the chemical modification may include all of the nucleic acid modifications described in (WO 2019089820 A1), but is not limited thereto.

Gene Editing Method Using Engineered crRNA

Overview

An aspect of the present disclosure provides a method for editing a target gene or a target nucleic acid in a target cell using an engineered crRNA. The target gene or the target nucleic acid includes a target sequence. The target nucleic acid may be single-stranded DNA, double-stranded DNA, and/or RNA. The gene editing method includes: delivering an engineered guide RNA, a Cas12f1 protein, or a nucleic acid encoding each of the engineered guide RNA and the Cas12f1 protein into a target cell including a target gene or a target nucleic acid. As a result, an engineered CRISPR/Cas12f1 complex is injected into the target cell, or the formation of the engineered CRISPR/Cas12f1 complex is induced, and a target gene is edited by the engineered CRISPR/Cas12f1 complex. The engineered guide RNA has the same features and structure as those described in the <Engineered guide RNA for CRISPR/Cas12f1 system> section. The Cas12f1 protein has the same features and configuration as the Cas12f1 protein and/or the modified Cas12f1 protein described in the <Engineered CRISPR/Cas12f1 complex> section.

In an embodiment, the gene editing method may include: delivering an engineered guide RNA or a nucleic acid encoding the engineered guide RNA, and a Cas12f1 protein or a nucleic acid encoding the Cas12f1 protein into a target cell. In this regard, the engineered guide RNA sequence includes a scaffold sequence, a spacer sequence, and a U-rich tail sequence. In this regard, the spacer sequence may complementarily bind to a target gene or a target nucleic acid included in the target cell. In an embodiment, the U-rich tail sequence may be represented by (UₐV)ₙU_{b}. In this regard, a, n, and b are an integer, a is 1 to 4, n is 0 or more, and b is 1 to 10 inclusive. In an embodiment, the U-rich tail sequence may be represented by (UₐN)ₙU_{b}. In this regard, N is one of adenosine (A), uridine (U), cytidine (C), and guanosine (G). In this regard, a is an integer between 1 to 4 inclusive, n is an integer selected from 0, 1, and 2, and b is an integer between 1 to 10 inclusive.

Target Cell

In an embodiment, the target cell may be a prokaryotic cell. In an embodiment, the target cell may be a eukaryotic cell. Specifically, the eukaryotic cell may be a plant cell, an animal cell, and/or a human cell, but is not limited thereto.

Determination of Target Sequence

A target gene or nucleic acid and a target sequence to be edited using a CRISPR Cas12f1 complex may be determined in consideration of the purpose of gene editing, the target cell environment, the PAM sequence recognized by the Cas12f1 protein, and/or other variables. In this regard, the method is not particularly limited as long as a target sequence having an appropriate length can be determined, and a known technique may be utilized.

Determination of Spacer Sequence According to Target Sequence

Once the target sequence is determined, the corresponding spacer sequence is designed. The spacer sequence is designed as a sequence capable of complementarily binding to the target sequence. In an embodiment, the spacer sequence is designed as a sequence capable of complementarily binding to the target gene. In an embodiment, the spacer sequence is designed so as to be capable of complementarily binding to the target nucleic acid. In an embodiment, the spacer sequence is designed as a sequence which is complementary to a target sequence included in a target strand sequence of the target nucleic acid. In an embodiment, the spacer sequence is designed as an RNA sequence corresponding to a DNA sequence of a protospacer included in a non-target strand sequence of the target nucleic acid. Specifically, the spacer sequence has the same base sequence as the protospacer sequence, but is designed as a sequence in which each of the thymidines included in the base sequence is substituted with uridine.

Complementarity of Target Sequence and Spacer Sequence

In an embodiment, the spacer sequence may be a sequence which is 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to the target sequence. In an embodiment, the spacer sequence may be a sequence which is complementary to the target sequence within the numerical range selected in the immediately preceding sentence. For example, the spacer sequence may be a sequence which is 60% to 90% complementary to the target sequence. In an embodiment, the spacer sequence may be a sequence which is 90% to 100% complementary to the target sequence.

Number of Mismatches Between Target Sequence and Spacer Sequence

In an embodiment, the spacer sequence may be a complementary sequence with 0, 1, 2, 3, 4, 5, 6, 7, 8, 9, or 10 mismatches with the target sequence. In an embodiment, the spacer sequence may have mismatches within the numerical range of two of these values. For example, the spacer sequence may have 1 to 5 mismatches with the target sequence. In an embodiment, the spacer sequence may have 6 to 10 mismatches with the target sequence.

Use of CRISPR/Cas12f1 Complex

The gene editing method provided in the present disclosure uses the fact that the engineered CRISPR/Cas12f1 complex has the activity of cleaving a gene or a nucleic acid in a target-specific manner. The engineered CRISPR/Cas12f1 complex has the same features and configuration as the engineered CRISPR/Cas12f1 complex described in the <Engineered CRISPR/Cas12f1 complex> section.

Delivery of Each Component of CRISRP/Cas12f1 Complex into Cell

In the gene editing method provided in the present disclosure, it is assumed that a CRISPR/Cas12f1 complex engineered within the target cell is brought into contact with a target gene or nucleic acid. Therefore, in order to induce contact of the engineered CRISPR/Cas12f1 complex with the target gene or the target nucleic acid, the gene editing method includes: delivering each component of the engineered CRISPRCas12f1 complex into a target cell. In an embodiment, the gene editing method may include delivering an engineered guide RNA or a nucleic acid encoding the engineered guide RNA and a Cas12f1 protein or a nucleic acid encoding the Cas12f1 protein into a target cell. In an embodiment, the gene editing method may include delivering an engineered guide RNA and a Cas12f1 protein into a target cell. In an embodiment, the gene editing method may include delivering a nucleic acid encoding an engineered guide RNA and a Cas12f1 protein into a target cell. In an embodiment, the gene editing method may include delivering an engineered guide RNA and a nucleic acid encoding a Cas12f1 protein into a target cell. In an embodiment, the gene editing method may include delivering a nucleic acid encoding an engineered guide RNA and a nucleic acid encoding a Cas12f1 protein into a target cell. An engineered guide RNA or a nucleic acid encoding the engineered guide RNA and a Cas12f1 protein or a nucleic acid encoding the Cas12f1 protein may be delivered in various delivery forms into a target cell using various delivery methods.

Delivery Form—RNP

As a delivery form, ribonucleoprotein particles (RNPs) in which an engineered guide RNA and a Cas12f1 protein are combined may be used. In an embodiment, the gene editing method may include injecting a CRISPR/Cas12f1 complex in which an engineered guide RNA and a Cas12f1 protein are combined into a target cell.

Delivery Form—Non-Viral Vector

As another delivery form, a non-viral vector including a nucleic acid sequence encoding an engineered guide RNA and a nucleic acid sequence encoding a Cas12f1 protein may be used. In an embodiment, the gene editing method may include injecting a non-viral vector including a nucleic acid sequence encoding an engineered guide RNA and a nucleic acid sequence encoding a Cas12f1 protein into a target cell. Specifically, the non-viral vector may be a plasmid, naked DNA, a DNA complex, or mRNA, but is not limited thereto. In an embodiment, the gene editing method may include injecting a first non-viral vector including a nucleic acid sequence encoding an engineered guide RNA and a second non-viral vector including a nucleic acid sequence encoding a Cas12f1 protein into a target cell. Specifically, the first non-viral vector and the second non-viral vector may be each one selected from the group consisting of a plasmid, naked DNA, a DNA complex, and mRNA, but are not limited thereto.

Delivery Form—Viral Vector

As still another delivery form, a viral vector including a nucleic acid sequence encoding an engineered guide RNA and a nucleic acid sequence encoding a Cas12f1 protein may be used. In an embodiment, the gene editing method may include: injecting a nucleic acid sequence encoding an engineered guide RNA and a nucleic acid sequence encoding a Cas12f1 protein into a target cell. Specifically, the viral vector may be one selected from the group consisting of a retrovirus, a lentivirus, an adenovirus, an adeno-associated virus (AAV), a vaccinia virus, a poxvirus, and a herpes simplex virus, but is not limited thereto. In an embodiment, the viral vector may be adeno-associated virus (AAV).

In an embodiment, the gene editing method may include: injecting a first viral vector including a nucleic acid sequence encoding an engineered guide RNA and a second viral vector including a nucleic acid sequence encoding a Cas12f1 protein into a target cell. Specifically, the first viral vector and the second viral vector may be each one selected from the group consisting of a retrovirus, a lentivirus, an adenovirus, an adeno-associated virus (AAV), a vaccinia virus, a poxvirus and a herpes simplex virus, but are not limited thereto.

Delivery Method—General Delivery Means

The delivery method is not particularly limited as long as it can deliver an engineered guide RNA or a nucleic acid encoding the engineered guide RNA, and a Cas12f1 protein or a nucleic acid encoding the Cas12f1 protein in an appropriate delivery form into a cell. In an embodiment, the delivery method may be electroporation, a gene gun method, ultrasonic perforation, magnetofection, and/or transient cell compression or squeezing.

Delivery Method—Nanoparticles

The delivery method may be delivering at least one component included in the CRISPR/Cas12f1 system using nanoparticles. In this regard, the delivery method may be a known method which can be appropriately selected by those skilled in the art. For example, the nanoparticle delivery method may be the method disclosed in (WO 2019/089820A1), but is not limited thereto.

In an embodiment, the delivery method may be delivering a Cas12f1 protein or a nucleic acid encoding the Cas12f1 protein and/or an engineered guide RNA or a nucleic acid encoding the engineered guide RNA using nanoparticles. In an embodiment, the delivery method may be delivering a Cas12f1 protein or a nucleic acid encoding the Cas12f1 protein, a first engineered guide RNA or a nucleic acid encoding the first engineered guide RNA and/or a second engineered guide RNA or a nucleic acid encoding the second engineered guide RNA using nanoparticles. In this regard, the delivery method may be a cationic liposome method, a lithium acetate-DMSO method, lipid-mediated transfection, a calcium phosphate precipitation method, lipofection, polyethyleneimine (PEI)-mediated transfection, DEAE-dextran-mediated transfection, and/or nanoparticle-mediated nucleic acid delivery (see Panyam et., al Adv Drug Deliv Rev. 2012 Sep. 13. pii: 50169-409X(12)00283-9. doi: 10.1016/j.addr.2012.09.023), but is not limited thereto. In this regard, the component of the CRISPR/Cas12f1 system may be in the form of a RNP, a non-viral vector, and/or a viral vector. For example, the component of the CRISPR/Cas12f1 system may be in the form of mRNA encoding each component, but is not limited thereto.

Delivery Form and Method—Possible Combination

The gene editing method includes: delivering an engineered guide RNA or a nucleic acid encoding the engineered guide RNA, and a Cas12f1 protein or a nucleic acid encoding the Cas12f1 protein into a cell, and in this regard, the delivery form and/or delivery method of the components may be the same as or different from each other. In an embodiment, the gene editing method may include: delivering an engineered guide RNA or a nucleic acid encoding the engineered guide RNA in a first delivery form and delivering a Cas12f1 protein or a nucleic acid encoding the Cas12f1 protein in a second delivery form. In this regard, the first delivery form and the second delivery form may be each any one of the above-described delivery forms. In an embodiment, the gene editing method may include: delivering an engineered guide RNA or a nucleic acid encoding the engineered guide RNA by a first delivery method, and delivering a Cas12f1 protein or a nucleic acid encoding the Cas12f1 protein by a second delivery method. In this regard, the first delivery method and the second delivery method may be each any one of the above-described delivery methods.

Delivery Order

The gene editing method includes: delivering an engineered guide RNA or a nucleic acid encoding the engineered guide RNA, and a Cas12f1 protein or a nucleic acid encoding the Cas12f1 protein into a cell, and in this regard, the components may be simultaneously delivered into the cell, but may be sequentially delivered with a time interval there between.

In an embodiment, the gene editing method may include: simultaneously delivering an engineered guide RNA or a nucleic acid encoding the engineered guide RNA and a Cas12f1 protein or a nucleic acid encoding the Cas12f1 protein. In an embodiment, the gene editing method may include: delivering an engineered guide RNA or a nucleic acid encoding the engineered guide RNA into the cell, and then delivering a Cas12f1 protein or a nucleic acid encoding the Cas12f1 protein into the cell after a time interval. In an embodiment, the gene editing method may include: delivering a Cas12f1 protein or a nucleic acid encoding the Cas12f1 protein into the cell, and then delivering an engineered guide RNA into the cell after a time interval. In an embodiment, the gene editing method may include: delivering a nucleic acid encoding a Cas12f1 protein into the cell, and then delivering an engineered guide RNA in the cell after a time interval.

Delivery of a Plurality of Engineered Guide RNAs or Nucleic Acid Encoding the Plurality of Engineered Guide RNAs

The gene editing method provided in the present disclosure may include delivering a Cas12f1 protein or a nucleic acid encoding the Cas12f1 protein, and two or more engineered guide RNAs or a nucleic acid encoding the two or more engineered guide RNAs into a target cell. By the method, two or more CRISPR/Cas12f1 complexes targeting different sequences may be injected into a target cell, or may be formed in the target cell. As a result, two or more target genes or target nucleic acids included in the cell may be edited. In an embodiment, the gene editing method include: delivering a Cas12f1 protein or a nucleic acid encoding the Cas12f1 protein, a first engineered guide RNA or a nucleic acid encoding the first engineered guide RNA, and a second engineered guide RNA or a nucleic acid encoding the second engineered guide RNA into a target cell including gene or nucleic acid. In this regard, each of the components may be delivered into the cell using one or more of the delivery forms and methods described above. In this regard, two or more components may be simultaneously delivered into the cell, or may be sequentially delivered into the cell with a time interval therebetween.

Bringing CRISPR/Cas12f1 Complex and Target Nucleic Acid in Contact with Each Other

In the gene editing method provided in the present disclosure, editing of a target gene or target nucleic acid is performed while the engineered CRISPR/Cas12f1 complex in the target cell is being brought into contact with the target gene or the target nucleic acid. Therefore, the gene editing method may include bringing an engineered CRISPR/Cas12f1 complex into contact in a target cell, or inducing the contact. In an embodiment, the gene editing method may include: bringing an engineered CRISPR/Cas12f1 complex into contact with a target nucleic acid in a target cell. In an embodiment, the gene editing method may include: inducing a contact of an engineered CRISPR/Cas12f1 complex with a target nucleic acid in a target cell. In this regard, the induction is not particularly limited as long as it is a method for bringing the engineered CRISPR/Cas12f1 complex into contact with the target nucleic acid in the cell. In an embodiment, the induction may include: delivering an engineered guide RNA or a nucleic acid encoding the engineered guide, and a Cas12f1 protein or a nucleic acid encoding the Cas12f1 protein into the cell.

Gene Editing Results—Indel

As a result of performing the gene editing method provided in the present disclosure, an indel may be generated in a target gene or target nucleic acid. In this regard, the indel may occur inside and/or outside a target sequence portion and/or a protospacer sequence portion. The indel refers to a mutation in a nucleotide sequence of a nucleic acid, in which a part of the nucleotide sequence is deleted, an arbitrary nucleotide is inserted, and/or the insertion and the deletion are combined from the pre-edited sequence. In general, the occurrence of an indel in the target gene or the target nucleic acid sequence inactivates the corresponding gene or nucleic acid. In an embodiment, as a result of performing the gene editing method, one or more nucleotides in the target gene or the target nucleic acid may be deleted and/or added.

Gene Editing Results—Base Editing

As a result of performing the gene editing method provided in the present disclosure, base editing may occur within the target gene or the target nucleic acid. This means that unlike an indel, in which a target gene or any base in the target nucleic acid is deleted or added, one or more specific bases in the nucleic acid are changed as intended. In other words, base editing causes a predetermined point mutation at a specific position in the target gene or nucleic acid. In an embodiment, as a result of performing the gene editing method, one or more bases in the target gene or the target nucleic acid may be substituted with other bases.

Gene Editing Results—Insertion

As a result of performing the gene editing method provided in the present disclosure, a knock-in may occur in the target gene or the target nucleic acid. The knock-in means inserting an additional nucleic acid sequence into the target gene or the target nucleic acid sequence. In addition to the CRISPR/Cas12f1 complex, a donor including the additional nucleic acid sequence is further required for the knock-in to occur. When the CRISPR/Cas12f1 complex cleaves a target gene or target nucleic acid in the cell, repair of the cleaved target gene or target nucleic acid will occur. In this regard, the donor is involved in the repair process, so that the additional nucleic acid sequence is inserted into the target gene or the target nucleic acid. In an embodiment, the gene editing method may further include: delivering a donor into a target cell. In this regard, the donor includes an additional target nucleic acid, and the donor induces the insertion of the additional target nucleic acid into the target gene or the target nucleic acid. In this regard, when the donor is delivered into a target cell, the above-described delivery form and/or delivery method may be used.

Gene Editing Results—Deletion

As a result of performing the gene editing method provided in the present disclosure, all or part of the target gene or the target nucleic acid sequence may be deleted. The deletion means deleting a partial base sequence in the target gene or the target nucleic acid. In an embodiment, the gene editing method includes: introducing a Cas12f1 protein or a nucleic acid encoding the Cas12f1 protein, a first engineered guide RNA or a nucleic acid encoding the first engineered guide RNA, and a second engineered guide RNA or a nucleic acid encoding the second engineered guide RNA into a cell including a target gene or target nucleic acid. Accordingly, the deletion of the specific sequence portion in the target gene or the target nucleic acid occurs as a result of the gene editing.

Example of Gene Editing Method

In an embodiment, the gene editing method may include: delivering a CRISPR/Cas12f1 complex in the form of ribonucleoprotein particles which an engineered guide RNA and a Cas12f1 protein are combined into eukaryotic cells. In this regard, the delivery may be performed by electroporation or lipofection.

In an embodiment, the gene editing method may include: delivering a nucleic acid encoding an engineered guide RNA and a nucleic acid encoding a Cas12f1 protein into eukaryotic cells. In this regard, the delivery may be performed by electroporation or lipofection.

In an embodiment, the gene editing method may include: delivering an adeno-associated virus (AAV) vector including a nucleic acid sequence encoding an engineered guide RNA and a nucleic acid sequence encoding a Cas12f1 protein into eukaryotic cells.

In an embodiment, the gene editing method may include: delivering an adeno-associated virus (AAV) including a nucleic acid sequence encoding a first engineered guide RNA, a nucleic acid sequence encoding a second engineered guide RNA, and a nucleic acid sequence encoding a Cas12f1 protein into eukaryotic cells. Use of engineered crRNA

Gene editing use of engineered crRNA In an embodiment, the engineered crRNA may be used for gene editing of a target gene or nucleic acid including a target sequence.

In an embodiment, the engineered guide RNA may be used for gene editing of a target gene or nucleic acid including a target sequence.

In an embodiment, the engineered CRISPR/Cas12f1 complex may be used for gene editing of a target gene or nucleic acid including a target sequence.

In this regard, the engineered crRNA, the engineered guide RNA, and the engineered CRISPR/Cas12f1 complex have the same features and configuration as those described in each section.

In an embodiment, the use of gene editing of the target gene or nucleic acid may be used for performing the method described in the <Gene editing method using engineered crRNA> section.

In an embodiment, the use may include: delivering an engineered guide RNA or a nucleic acid encoding the engineered guide RNA, and a Cas12f1 protein or a nucleic acid encoding the Cas12f1 protein into a target cell. In this regard, the engineered guide RNA sequence includes a scaffold sequence, a spacer sequence, and a U-rich tail sequence. In this regard, the spacer sequence may complementarily bind to a target gene or a target nucleic acid included in the target cell. In an embodiment, the U-rich tail sequence may be represented by (UₐV)ₙU_{b}. In this regard, a, n, and b are an integer, a is 1 to 4 inclusive, n is 0 or more, and b is 1 to 10 inclusive. In an embodiment, the U-rich tail sequence may be represented by (UₐN)ₙU_{b}. In this regard, N is one of adenosine (A), uridine (U), cytidine (C), and guanosine (G). In this regard, a is an integer between 1 to 4 inclusive, n is an integer selected from 0, 1, and 2, and b is an integer between 1 to 10 inclusive.

In an embodiment, the use may include bringing an engineered CRISPR/Cas12f1 complex into contact with a target nucleic acid in a target cell.

In this regard, the engineered CRISPR/Cas12f1 complex has the same features and structure as those described in the <Engineered CRISPR/Cas12f1 complex> section.

In an embodiment, the use may include: inducing a contact of an engineered CRISPR/Cas12f1 complex with a target nucleic acid in a target cell. In this regard, the induction is not particularly limited as long as it is a method for bringing the engineered CRISPR/Cas12f1 complex into contact with the target nucleic acid in the cell. In an embodiment, the induction may include: delivering an engineered guide RNA or a nucleic acid encoding the engineered guide, and a Cas12f1 protein or a nucleic acid encoding the Cas12f1 protein into the cell.

Use of Engineered crRNA for Preparing Composition for Gene Editing

In an embodiment, an engineered crRNA may be used for preparing a composition for gene editing.

In an embodiment, an engineered guide RNA may be used for preparing a composition for gene editing.

In an embodiment, an engineered CRISPR/Cas12f1 complex may be used as a use for preparing a composition for gene editing.

In an embodiment, the composition for gene editing may include a Cas12f1 protein or a nucleic acid encoding the Cas12f1 protein, an engineered crRNA or a nucleic acid encoding the engineered crRNA, and a tracrRNA or a nucleic acid encoding the tracrRNA.

In an embodiment, the composition for gene editing may include a Cas12f1 protein or a nucleic acid encoding the Cas12f1 protein, and an engineered guide RNA or a nucleic acid encoding the engineered guide RNA.

In an embodiment, the composition for gene editing may include a CRISPR/Cas12f1 complex.

In an embodiment, the composition for gene editing may include a vector having a nucleic acid sequence encoding a Cas12f1 protein and a nucleic acid sequence encoding an engineered guide RNA.

[Example Mode of the Invention]

Hereinafter, the invention provided in the present disclosure will be described in more detail through Experimental Examples and Examples. These Examples are only for exemplifying the content disclosed by the present disclosure, and it will be obvious to those skilled in the art that the scope of the content disclosed by the present disclosure should not be interpreted as being limited by these Examples.

### Experimental Example 1 Preparation of Experimental Materials and Experimental Methods

### Experimental Example 1-1 Human Codon-Optimized Cas12f1 Sequence

A Cas12f1 protein belonging to the Cas14 family (hereinafter, Cas14a1, Harrington et al., Programmed DNA destruction by miniature CRISPR-Cas14 enzymes, Science 362, 839-842 (2018)) was codon-optimized using a codon optimization program, and a sequence in which NLS sequence was added to the 5′-end and 3′-terminus was gene-synthesized to secure CDS. PCR amplification was performed using the synthesized gene as a template, and cloning was performed according to a desired cloning sequence for a vector having a promoter capable of expression in a eukaryotic system and a polyA signal sequence by the Gibson assembly method. The sequence of a plasmid vector obtained after cloning was completed was finally confirmed by the Sanger sequencing method. The amino acid sequence of the Cas14a1 protein and the human codon-optimized nucleic acid sequence encoding the Cas14a1 protein are shown in Table 1.

TABLE 1
Sequence information of NLS-Cas14a1-NLS protein

| | | SEQ ID |
|---|---|---|
| Subject | Sequence information | NO |
| Cas12f1 | PKKKRKVGIHGVPAAMAKNTITKTLKLRIVRPYNSAEVEKIVADEKNNREK | 281 |
| (Amino | IALEKNKDKVKEACSKHLKVAAYCTTQVERNACLFCKARKLDDKFYQKLRG | |
| acid) | QFPDAVFWQEISEIFRQLQKQAAEIYNQSLIELYYEIFIKGKGIANASSVE | |
| | HYLSDVCYTRAAELFKNAAIASGLRSKIKSNFRLKELKNMKSGLPTTKSDN | |
| | FPIPLVKQKGGQYTGEEISNHNSDFIIKIPFGRWQVKKEIDKYRPWEKFDF | |
| | EQVQKSPKPISLLLSTQRRKRNKGWSKDEGTEAEIKKVMNGDYQTSYIEVK | |
| | RGSKIGEKSAWMLNLSIDVPKIDKGVDPSIIGGIDVGVKSPLVCAINNAFS | |
| | RYSISDNDLFHFNKKMFARRRILLKKNRHKRAGHGAKNKLKPITILTEKSE | |
| | RFRKKLIERWACEIADFFIKNKVGTVQMENLESMKRKEDSYFNIRLRGFWP | |
| | YAEMQNKIEFKLKQYGIEIRKVAPNNTSKTCSKCGHLNNYFNFEYRKKNKF | |
| | PHFKCEKCNFKENADYNAALNISNPKLKSTKEEPKRPAATKKAGQAKKKK | |
| Cas12f1 | ccaaagaagaagcggaaggtcggtatccacggagtcccagcagccATGGCC | 1 |
| (Human | AAGAACACAATTACAAAGACACTGAAGCTGAGGATCGTGAGACCATACAAC | |
| codon- | AGCGCTGAGGTCGAGAAGATTGTGGCTGATGAAAAGAACAACAGGGAAAAG | |
| optimized | ATCGCCCTCGAGAAGAACAAGGATAAGGTGAAGGAGGCCTGCTCTAAGCAC | |
| nucleic | CTGAAAGTGGCCGCCTACTGCACCACACAGGTGGAGAGGAACGCCTGTCTG | |
| acid) | TTTTGTAAAGCTCGGAAGCTGGATGATAAGTTTTACCAGAAGCTGCGGGGC | |
| | CAGTTCCCCGATGCCGTCTTTTGGCAGGAGATTAGCGAGATCTTCAGACAG | |
| | CTGCAGAAGCAGGCCGCCGAGATCTACAACCAGAGCCTGATCGAGCTCTAC | |
| | TACGAGATCTTCATCAAGGGCAAGGGCATTGCCAACGCCTCCTCCGTGGAG | |
| | CACTACCTGAGCGACGTGTGCTACACAAGAGCCGCCGAGCTCTTTAAGAAC | |
| | GCCGCTATCGCTTCCGGGCTGAGGAGCAAGATTAAGAGTAACTTCCGGCTC | |
| | AAGGAGCTGAAGAACATGAAGAGCGGCCTGCCCACTACAAAGAGCGACAAC | |
| | TTCCCAATTCCACTGGTGAAGCAGAAGGGGGGCCAGTACACAGGGTTCGAG | |
| | ATTTCCAACCACAACAGCGACTTTATTATTAAGATCCCCTTTGGCAGGTGG | |
| | CAGGTCAAGAAGGAGATTGACAAGTACAGGCCCTGGGAGAAGTTTGATTTC | |
| | GAGCAGGTGCAGAAGAGCCCCAAGCCTATTTCCCTGCTGCTGTCCACACAG | |
| | CGGCGGAAGAGGAACAAGGGGTGGTCTAAGGATGAGGGGACCGAGGCCGAG | |
| | ATTAAGAAAGTGATGAACGGCGACTACCAGACAAGCTACATCGAGGTCAAG | |
| | CGGGGCAGTAAGATTGGCGAGAAGAGCGCCTGGATGCTGAACCTGAGCATT | |
| | GACGTGCCAAAGATTGATAAGGGCGTGGATCCCAGCATCATCGGAGGGATC | |
| | GATGTGGGGGTCAAGAGCCCCCTCGTGTGCGCCATCAACAACGCCTTCAGC | |
| | AGGTACAGCATCTCCGATAACGACCTGTTCCACTTTAACAAGAAGATGTTC | |
| | GCCCGGCGGAGGATTTTGCTCAAGAAGAACCGGCACAAGCGGGCCGGACAC | |
| | GGGGCCAAGAACAAGCTCAAGCCCATCACTATCCTGACCGAGAAGAGCGAG | |
| | AGGTTCAGGAAGAAGCTCATCGAGAGATGGGCCTGCGAGATCGCCGATTTC | |
| | TTTATTAAGAACAAGGTCGGAACAGTGCAGATGGAGAACCTCGAGAGCATG | |
| | AAGAGGAAGGAGGATTCCTACTTCAACATTCGGCTGAGGGGGTTCTGGCCC | |
| | TACGCTGAGATGCAGAACAAGATTGAGTTTAAGCTGAAGCAGTACGGGATT | |
| | GAGATCCGGAAGGTGGCCCCCAACAACACCAGCAAGACCTGCAGCAAGTGC | |
| | GGGCACCTCAACAACTACTTCAACTTCGAGTACCGGAAGAAGAACAAGTTC | |
| | CCACACTTCAAGTGCGAGAAGTGCAACTTTAAGGAGAACGCCGATTACAAC | |
| | GCCGCCCTGAACATCAGCAACCCTAAGCTGAAGAGCACTAAGGAGGAGCCC | |
| | aaaaggccggcggccacgaaaaaggccggccaggcaaaaaagaaaaag | |

### Experimental Example 1-2 Preparation of Plasmid Vector

An oligonucleotide (Bionics) including a human codon-optimized Cas12f1 DNA sequence used in this experiment was synthesized. An oligonucleotide for the Cas12f1 protein sequence was cloned into a plasmid including a Chicken β-actin promoter, NLS at the 5′ end and 3′ end, and T2A-linked eGFP. A template DNA for a canonical guide RNA used in this experiment was synthesized (Twist Bioscience) and cloned into a pTwist Amp plasmid for replication. A template DNA for an engineered guide RNA was constructed using an enzyme cloning technique and cloned into a pTwist Amp plasmid for replication. By using the plasmid as a template if necessary, an amplicon of a guide RNA or engineered guide RNA was prepared using a U6-complementary forward primer and a protospacer sequence-complementary reverse primer. Further, the prepared amplicon was cloned into a T-blunt plasmid (Biofact) for replication, if necessary. In order to prepare a (engineered) dual guide RNA, oligonucleotides encoding a tracrRNA and a (engineered) crRNA were replicated within pSilencer 2.0 (ThermoFisher Scientific) using BamHI and HindIII restriction enzymes (New England Biolabs).

### Experimental Example 1-3 Preparation of Viral Vector

An adeno-associated virus (AAV) inverted terminal repeat vector including a guide RNA (or an engineered guide RNA) sequence and a Cas12f1 protein sequence linked to an enhanced green fluorescent protein (eGFP) through an NLS and a self-cleaving T2A sequence was prepared. The transcription of Cas12f1 and a guide RNA was promoted by a chicken β-actin and a U6 promoter, respectively. To prepare a rAAV2 vector, pAAV-ITR-sgRNA-Cas12f1, pAAVED29 and a helper plasmid were transfected into HEK293T cells. The transfected HEK293T cells were cultured in a DMEM medium containing 2% FBS. A recombinant pseudotyped AAV vector stock was produced using Polyplus-transfection (PEIpro) and PEI coprecipitation using triple-transfection for the plasmid at the same molar ratio. After being cultured for 72 hours, the cells were lysed and the lysate was purified by iodixanol (Sigma-Aldrich) stepgradient ultracentrifugation.

### Experimental Example 1-4 Preparation of Guide RNA Amplicon

In order to prepare a guide RNA and an engineered guide amplicon, PCR amplification was performed using a U6-complementary forward primer and a protospacer sequence-complementary reverse primer for a template DNA plasmid for a canonical guide RNA and an engineered guide RNA template DNA plasmid, using KAPA HiFi HotStart DNA polymerase (Roche) or Pfu DNA polymerase (Biofact). The PCR amplification product was purified using the Higene Gel&PCR purification system (Biofact) to obtain guide RNA and engineered guide RNA amplicons.

### Experimental Example 1-5 Preparation of Guide RNA

One of the following methods was used to prepare a guide RNA (or an engineered guide RNA).

A guide RNA was prepared by chemically synthesizing a pre-designed guide RNA.

A PCR amplicon including a pre-designed guide RNA sequence and a T7 promoter sequence was prepared. In vitro transcription was performed using a NEB T7 polymerase with the PCR amplicon as a template. After the resulting product obtained by performing the in vitro transcription was treated with NEB DNase I, the treated product was purified using a Monarch RNA Cleanup Kit (NEB), and then a guide RNA was obtained.

A plasmid vector including a pre-designed guide RNA sequence and a T7 promoter sequence was prepared by the Tblunt plasmid cloning method in Experimental Example 1-2. After the vector was purified by double-cutting both ends of a guide RNA sequence including a T7 promoter sequence, in vitro transcription was performed on the resulting product using NEB T7 polymerase. After the resulting product obtained by performing the in vitro transcription was treated with NEB DNase I, the treated product was purified using a Monarch RNA Cleanup Kit (NEB), and then a guide RNA was obtained.

### Experimental Example 1-6 Preparation of Recombinant Cas12f1 Protein

The human codon-optimized Cas12f1 DNA sequence used in this experiment was replicated in a pMAL-c2 plasmid vector. The plasmid vector was used for transformation of BL21 (DE3) E. coli cells. The transformed E. coli colonies were grown in LB broth at 37° C. until the optical density reached 0.7. The transformed E. coli cells were cultured at 18° C. overnight in the presence of 0.1 mM isopropylthio-β-D-galactoside. The cells were then centrifuged at 3,500 g for 30 minutes and collected. The cells were resuspended in 20 mm Tris-HCl (pH 7.6), 500 mM NaCl, 5 mM β-mercaptoethanol, 5% glycerol. The cells were lysed and the lysed cells were disrupted by sonication. After a sample including the disrupted cells was centrifuged at 15,000 g for 30 minutes, the supernatant was filtered through a 0.4-μm syringe filter (Millipore). The filtered supernatant was loaded onto a Ni²⁺-affinity column using a FPLC purification system (ÄKT Aurora, GE Healthcare). The bound fractions were eluted at a gradient of 80-400 mM imidazole, 20 mM Tris-HCl (pH 7.5). The eluted protein was treated with TEV protease for 16 hours. The cleaved protein was purified in a Heparin column at a 0.15-1.6 M NaCl linear concentration gradient. A recombinant Cas12f1 protein purified in the Heparin column was dialyzed against 20 mM Tris pH 7.6, 150 mM NaCl, 5 mM β-mercaptoethanol, and 5% glycerol. The dialyzed proteins were purified by passing through an MBP column, and then re-purified in a monoS column (GE Healthcare) or Enrich S at a linear gradient of 0.5-1.2 M NaCl. The re-purified proteins were collected and dialyzed against 20 mm Tris pH 7.6, 150 mM NaCl, 5 mM β-mercaptoethanol, and 5% glycerol to prepare a Cas12f1 protein. The concentration of the protein produced was quantified by Bradford quantification using bovine serum albumin as a standard and measured electrophoretically on a Coomassie blue-stained SDS-PAGE gel.

### Experimental Example 1-7 Preparation of RNP

Ribonucleoprotein particles (RNPs) were prepared by incubating the recombinant Cas12f1 protein prepared according to Experimental Example 1-6 and the guide RNA (or engineered guide RNA) prepared according to Experimental Example 1-5 at 300 nM and 900 nM, respectively at room temperature for 10 minutes.

### Experimental Example 1-8 Cell Culture

HEK-293T cells (ATCC CRL-11269) were cultured in a DMEM medium under the conditions of 37° C. and 5% CO₂ with the addition of 10% heat-inactivated FBS (Corning) and 1% penicillin/streptomycin.

### Experimental Example 1-9 Transfection of Plasmid Vector or PCR Amplicon in Cells

Cell transfection was performed by electroporation or lipofection. In the case of electroporation, 4×10⁵ HEK-293 T cells were transfected with 2 to 5 μg of each of a plasmid vector encoding the Cas12f1 protein prepared in Experimental Example 1-2 and DNA encoding a guide RNA (and an engineered guide RNA) using a Neon transfection system (Invitrogen). The electroporation was performed under the conditions of 1300 V, 10 mA, and 3 pulses. For lipofection, 3 to 15 μL of FuGene reagent (Promega) was mixed with 1 to 5 μg of a plasmid vector encoding a Cas12f1 protein and 0.3 to 2 μg of a PCR amplicon for 15 minutes. The mixture was added to 1 ml of a DMEM medium plated with 1×10⁶ cells on day 1 prior to transfection. The cells were cultured in the presence of the mixture for 72 to 96 hours. After the culture, the cells were collected and the genomic DNA of the cells was manually isolated using a PureHelix™ genomic DNA preparation kit (NanoHelix) or a Maxwell RSC Cultured cells DNA Kit (Promega).

### Experimental Example 1-10 Intracellular RNP Transfection

The ribonucleoprotein particles (RNPs) prepared in Experimental Example 1-7 were transfected using the electroporation in Experimental Example 1-9, or the plasmid encoding the Cas12f1 protein prepared in Experimental Example 1-2 was transfected by the lipofection method in Experimental Example 1-9 on day 1 prior to the transfection of a guide RNA (and an engineered guide RNA) and the guide RNA (and the engineered guide RNA) prepared by Experimental Example 1-5 was transfected using the electroporation in Experimental Example 1-9 after one day.

### Experimental Example 1-11 Transfection of Viral Vector in Cells

Human HEK293T cells were infected with rAAV2-Cas12f1-sgRNA at different multiplicities of infection (MOIs) of 1, 5, 10, 50, and 100 determined by quantitative PCR. The transfected HEK293T cells were cultured in a DMEM medium containing 2% FBS. At different time points, cells were collected to isolate genomic DNA.

### Experimental Example 1-12 Quantitative Real-Time PCR (Quantitative rtPCR)

A guide RNA (or an engineered guide RNA) or a genomic DNA was each extracted from HEK293 T cells using RNeasy Miniprep kit (Qiagen) or Maxwell RSC miRNA Tissue Kit (Promega) or DNeasy Blood & Tissue Kit (Qiagen). To quantify the guide RNA, a RNA-specific primer was ligated and cDNA was synthesized using a crRNA-specific primer. The cDNA was used as a template for quantitative real-time PCR. Real-time PCR was analyzed using the KAPA SYBR FASTqPCR Master Mix (2×) Kit (KapaBiosystems).

### Experimental Example 1-13 Western Blot for Confirming Cas Protein Expression

A plasmid vector encoding the Cas protein prepared by Experimental Example 1-2 was prepared. In this regard, the plasmid further has a sequence (5′-TACCCATACGATGTTCCAGATTACGCTTATCCCTACGACGTGCCTGATTATG CATACCCATATGATGTCCCCGACTATGCC-3′, SEQ ID NO: 315) encoding an HA tag such that the HA tag can be linked and expressed when the Cas protein is expressed in the cell. The HEK293T cells cultured in Experimental Example 1-8 were transfected with the plasmid vector by the method disclosed in Experimental Example 1-9. The HEK293T cells were inoculated into a DMEM medium containing penicillin/streptomycin and statically cultured in an incubator at 37° C. and 5% CO₂ incubator for 48 hours. Then, the HEK293T cells were diluted with PBS and then disrupted by sonication. After a sample containing disrupted cells was centrifuged at 15,000 g and 4° C. for 10 minutes, the supernatant was quantified by the Bradford quantification method. It was tested by western blot whether a quantified HA antibody was expressed in vivo.

### Experimental Example 1-14 Indel Efficiency Analysis

Among the genomic DNA isolated from HEK-293T cells, a region containing a protospacer was subjected to PCR using a target-specific primer in the presence of KAPA HiFi HotStart DNA polymerase (Roche). The amplification method followed the manufacturer's instructions. A PCR amplicon, which is the resulting product of the amplification including Illumina TruSeq HT dual indexes, was subjected to 150-bp paired end sequencing using Illumina iSeq 100. An indel frequency was calculated using MAUND.MAUND is provided at https://github.com/ibscge/maund.

### Experimental Example 1-15 Analysis of T7 Endonuclease I (T7E1)

A PCR product was obtained using BioFACT™ Lamp Pfu DNA polymerase. The PCR product (100 to 300 μg) was reacted with 10 units of T7E1 enzyme (New England Biolabs) in a 25 μg reaction mixture at 37° C. for 30 minutes. The 20 p1 reaction mixture was loaded directly onto a 10% arylamide gel and the cleaved PCR product was run in a TBE buffer system. The gel image was stained with an ethidium bromide solution, and then digitized using a Printgraph 2M gel imaging system (Atto). Gene editing efficiency was evaluated by analyzing the digitized result.

### Experimental Example 1-16 Statistical Analysis

Statistical significance verification by a two-tailed Student's t-test was performed by Sigma Plot software (ver. 14.0). When a p-value less than 0.05 appears, it was considered statistically significant, and the p-value is illustrated in each drawing. In a box plot and a dot plot, the data points indicate the full range of values, and each box spans the interquartile range (25 to 75 percentile). The median and mean values are indicated by parallel lines of black and red, respectively. Error bars for all dot plot and bar plot data are illustrated using Sigmaplot (v. 41.0) and mean the standard deviation value of each data. The sample size was not pre-determined based on statistical methods.

### Experimental Example 2 Indel Efficiency Comparison 1 of Engineered CRISPR/Cas12f1 System Having U-Rich Tail

Experiments were conducted to evaluate the indel efficiency of an engineered CRISPR/Cas12f1 system having a U-rich tail sequence, using various gene sequences as a target nucleic acid. The protospacer sequence of the target nucleic acid and each PAM sequence are disclosed in Table 2.

TABLE 2
Target nucleic acid of Experimental Example 2

| Target | | |
|---|---|---|
| nucleic | Protospacer sequence (5′ to 3′) | |
| acid | of target nucleic acid | PAM |
| RNF2 | CACGTCTCATATGCCCCTTG (SEQ ID NO: 62) | TTTA |
| DYtarget2 | CACACACACAGTGGGCTACC (SEQ ID NO: 63) | TTTG |
| DYtarget9 | GACGTGGGAGACACACACAC (SEQ ID NO: 64) | TTTA |
| DYtarget10 | CATCCCCAGGACACACACAC (SEQ ID NO: 65) | TTTG |
| DYtarget13 | AGCACATGTGCACACACACA (SEQ ID NO: 66) | TTTG |

1) A plasmid vector including a human codon-optimized sequence encoding a Cas12f1 protein was prepared according to Experimental Example 1-2.

2) An amplicon capable of expressing a wild-type, or (an engineered) single guide RNA having a U-rich tail sequence was prepared according to Experimental Example 1-4. DNA sequences encoding the single guide RNA and primer sequences used to prepare an amplicon are disclosed in Tables 3 to 5.

TABLE 3
DNA sequences encoding wild-type (WT) sgRNA used in Experimental Example 2

| | | sgRNA common | | U-rich tail | |
|---|---|---|---|---|---|
| | Target | sequence | Protospacer sequence | sequence | SEQ ID |
| Label | nucleic acid | (5′ to 3′) | (5′ to 3′) | (3′ to 5′) | NO |
| WT | RNF2 | CTTCACTGATAAAG | CACGTCTCATATGCCCCTTG | - | 127 |
| | | TGGAGAACCGCTTC | | | |
| | DYtarget2 | ACCAAAAGCTGTC | CACACACACAGTGGGCTACC | | 129 |
| | | CCTTAGGGGATTAG | | | |
| | DYtarget9 | AACTTGAGTGAAG | GACGTGGGAGACACACACAC | | 156 |
| | | GTGGGCTGCTTGCA | | | |
| | DYtarget10 | TCAGCCTAATGTCG | CATCCCCAGGACACACACAC | | 158 |
| | | AGAAGTGCTTTCTT | | | |
| | DYtarget13 | CGGAAAGTAACCC | AGCACATGTGCACACACACA | | 171 |
| | | TCGAAACAAATTC | | | |
| | | ATTTTTCCTCTCCA | | | |
| | | ATTCTGCACAAgaaa | | | |
| | | GTTGCAGAACCCG | | | |
| | | AATAGACGAATGA | | | |
| | | AGGAATGCAAC | | | |
| | | (SEQ ID NO: 126) | | | |

In the above table, for the DNA sequence encoding sgRNA for each target nucleic acid, an sgRNA common sequence—a protospacer sequence—a U-rich tail sequence are linked in the 5′-to-3′ direction, and a scaffold sequence in the sgRNA common sequence is a sequence portion of SEQ ID NO: 254. The amplicon sequence capable of expressing the (engineered) single guide RNA is an amplicon sequence in which a U6 promoter is operably linked to the DNA sequence encoding the sgRNA.

TABLE 4
DNA sequences encoding engineered sgRNA used in Experimental Example 2

| | Target | sgRNA common | | U-rich tail | |
|---|---|---|---|---|---|
| | nucleic | sequence | Protospacer sequence | sequence | SEQ ID |
| Label | acid | (5′ to 3′) | (5′ to 3′) | (3′ to 5′) | NO |
| Engi- | RNF2 | CTTCACTGATAAAG | CACGTCTCATATGCCCCTTG | TTTTATTTTTT | 128 |
| neered | | TGGAGAACCGCTTC | | | |
| sgRNA | DYtarget2 | ACCAAAAGCTGTC | CACACACACAGTGGGCTACC | | 142 |
| | | CCTTAGGGGATTAG | | | |
| | DYtarget9 | AACTTGAGTGAAG | GACGTGGGAGACACACACAC | | 157 |
| | | GTGGGCTGCTTGCA | | | |
| | DYtarget10 | TCAGCCTAATGTCG | CATCCCCAGGACACACACAC | | 159 |
| | | AGAAGTGCTTTCTT | | | |
| | DYtarget13 | CGGAAAGTAACCC | AGCACATGTGCACACACACA | | 172 |
| | | TCGAAACAAATTC | | | |
| | | ATTTTTCCTCTCCA | | | |
| | | ATTCTGCACAAgaaa | | | |
| | | GTTGCAGAACCCG | | | |
| | | AATAGACGAATGA | | | |
| | | AGGAATGCAAC | | | |
| | | (SEQ ID NO: 126) | | | |

In the above table, for the DNA sequence encoding sgRNA for each target nucleic acid, an sgRNA common sequence—a protospacer sequence—a U-rich tail sequence are linked in the 5′-to-3′ direction, and a scaffold sequence in the sgRNA common sequence is a sequence portion of SEQ ID NO: 254. The amplicon sequence capable of expressing the (engineered) single guide RNA is an amplicon sequence in which a U6 promoter is operably linked to the DNA sequence encoding the sgRNA.

TABLE 5
Primer sequences used in Experimental Example 2

| | Target | Forward primer | Reverse primer | SEQ ID |
|---|---|---|---|---|
| sgRNA | nucleic acid | (5′ to 3′) | (5′ to 3′) | NO |
| WT | RNF2 | GAGGGCCTATTT | CAAGGGGCATATGAGACGTGGTTGCATT | 185 |
| | | CCCATGATT | CCTTCATTCGTCTATTC | |
| | | (SEQ ID NO: | | |
| | DYtarget2 | 183) | GGTAGCCCACTGTGTGTGTGGTTGCATT | 187 |
| | | | CCTTCATTCGTCTATTC | |
| | DYtarget9 | | GTGTGTGTGTCTCCCACGTCGTTGCATTC | 214 |
| | | | CTTCATTCGTCTATTC | |
| | DYtarget10 | | GTGTGTGTGTCCTGGGGATGGTTGCATT | 216 |
| | | | CCTTCATTCGTCTATTC | |
| | DYtarget13 | | TGTGTGTGTGCACATGTGCTGTTGCATTC | 229 |
| | | | CTTCATTCGTCTATTC | |
| Engineered | RNF2 | | aaaaaataaaa | 186 |
| sgRNA | | | CAAGGGGCATATGAGACGTGGTTGCATT | |
| | | | CCTTCATTCGTCTATTC | |
| | DYtarget2 | | aaaaaataaaaGTAGCCCACTGTGTGTGTG | 200 |
| | | | GTTGCATTCCTTCATTCGTCTATTC | |
| | DYtarget9 | | aaaaaataaaaTGTGTGTGTCTCCCACGTC | 215 |
| | | | GTTGCATTCCTTCATTCGTCTATTC | |
| | DYtarget10 | | aaaaaataaaaTGTGTGTGTCCTGGGGATG | 217 |
| | | | GTTGCATTCCTTCATTCGTCTATTC | |
| | DYtarget13 | | aaaaaataaaaGTGTGTGTGCACATGTGCT | 230 |
| | | | GTTGCATTCCTTCATTCGTCTATTC | |

3) HEK-293T cells cultured according to Experimental Example 1-8 were transfected with a plasmid vector including the Cas12f1 sequence and an amplicon capable of expressing the single guide RNA by the method disclosed in Experimental Example 1-9.

4) The indel efficiencies for the target nucleic acids of each example were analyzed by the methods disclosed in Experimental Examples 1-12 to 1-15. The indel efficiency analysis results are shown in the following Table 6.

TABLE 6
Indel efficiency analysis results of Experimental Example 2

| Target nucleic acid | Control | WT | Engineered sgRNA |
|---|---|---|---|
| RNF2 | 0 | 0 | 0.029 |
| DYtarget2 | 0 | 0.028 | 0.749 |
| DYtarget9 | 0 | 0 | 0.238 |
| DYtarget10 | 0.029 | 0.031 | 1.076 |
| DYtarget13 | 0.529 | 0.526 | 1.176 |

In this regard, those transfected with only a plasmid vector including the Cas12f1 protein sequence without sgRNA were used as a control.

### Experimental Example 3 Indel Efficiency Comparison 2 of Engineered CRISPR/Cas12f1 System Having U-Rich Tail

Experiments were conducted to evaluate the indel efficiency of an engineered CRISPR/Cas12f1 system having a U-rich tail sequence, using the DYtarget2, DYtarget10, DYtarget13, and Intergenic-22 as a target nucleic acid. The protospacer sequence of the target nucleic acid and each PAM sequence are disclosed in Table 7.

TABLE 7
Target nucleic acid sequences used in Experimental Example 3

| Target | Protospacer sequence (5′ to 3′) | |
|---|---|---|
| nucleic acid | of target nucleic acid | PAM |
| Intergenic-22 | AGAACACATACCCCTGGGCC (SEQ ID NO: 67) | TTTA |
| DYtarget2 | CACACACACAGTGGGCTACC (SEQ ID NO: 63) | TTTG |
| DYtarget10 | CATCCCCAGGACACACACAC (SEQ ID NO: 65) | TTTG |
| DYtarget13 | AGCACATGTGCACACACACA (SEQ ID NO: 66) | TTTG |

1) A plasmid vector including a human codon-optimized Cas12f1 sequence was prepared according to Experimental Example 1-2.

2) An amplicon capable of expressing a wild-type, or (an engineered) single guide RNA having a U-rich tail sequence was prepared according to Experimental Example 1-2. DNA sequences encoding the single guide RNA and primer sequences used to prepare an amplicon are shown in Tables 8 and 9.

[Table 8]

DNA Sequences Encoding sgRNA of Experimental Example 3

In the above table, for the DNA sequence encoding sgRNA for each target nucleic acid, an sgRNA common sequence—a protospacer sequence—a U-rich tail sequence are linked in the 5′-to-3′ direction, and a scaffold sequence in the sgRNA common sequence is a sequence portion of SEQ ID NO: 254. The amplicon sequence capable of expressing the (engineered) single guide RNA is an amplicon sequence in which a U6 promoter is operably linked to the DNA sequence encoding the sgRNA.

TABLE 9
Primer sequences used in Experimental Example 3

| | Target | Forward | | SEQ |
|---|---|---|---|---|
| | nucleic | primer | Reverse primer | ID |
| Label | acid | (5′ to 3′) | (5′ to 3′) | NO |
| Inter22 | Intergenic-22 | GAGGGCC | GGCCCAGGGGTATGTGTTCTGTTGCATTCCT | 231 |
| | | TATTTCCC | | |
| Inter22- | Intergenic-22 | ATGATT | aaaaaaGGCCCAGGGGTATGTGTTCTGTTGCAT | 232 |
| U6 | | (SEQ ID | TCCT | |
| | | NO: 183) | | |
| Inter22- | Intergenic-22 | | aaaaaataaaaGGCCCAGGGGTATGTGTTCTGTTG | 233 |
| U4aU6 | | | CATTCCT | |
| DYtarget2 | DYtarget2 | | GGTAGCCCACTGTGTGTGTGGTTGCATTCCT | 187 |
| | | | TCATTCGTCTATTC | |
| DYtarget2 | DYtarget2 | | aaaaaataaaaGGTAGCCCACTGTGTGTGTGGTTG | 200 |
| U4aU6 | | | CATTCCTTCATTCGTCTATTC | |
| DYtarget10 | DYtarget10 | | GTGTGTGTGTCCTGGGGATGGTTGCATTCCT | 216 |
| | | | TCATTCGTCTATTC | |
| DYtarget10- | DYtarget10 | | aaaaataaaaGTGTGTGTGTCCTGGGGATGGTTGC | 217 |
| U4aU6 | | | ATTCCTTCATTCGTCaTATTC | |
| DYtarget13 | DYtarget13 | | TGTGTGTGTGCACATGTGCTGTTGCATTCCTT | 229 |
| | | | CATTCGTCTATTC | |
| DYtarget13- | DYtarget1 | | aaaaaataaaaTGTGTGTGTGCACATGTGCTGTTG | 230 |
| U4aU6 | 3 | | CATTCCTTCATTCGTCTATTC | |

3) HEK-293T cells cultured according to Experimental Example 1-8 were transfected with a plasmid vector including the Cas12f1 sequence and an amplicon of the single guide RNA by the method disclosed in Experimental Example 1-9.

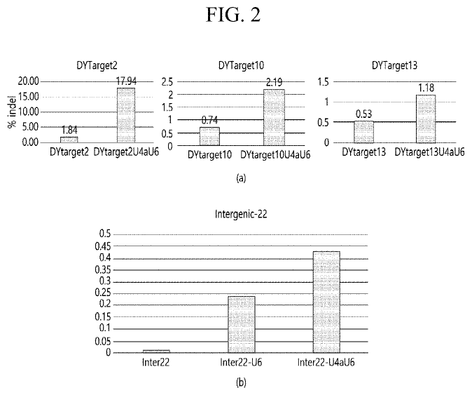

4) The indel efficiencies for the target nucleic acids of each example were analyzed by the methods disclosed in Experimental Examples 1-12 to 1-13. The indel efficiency analysis results are shown in FIG. 2.

As disclosed in FIG. 2, the CRISPR/Cas12f1 system including an engineered guide RNA with a U-rich tail sequence linked to the 3′-terminus thereof showed higher indel efficiency than a the wild-type.

### Experimental Example 4 Indel Efficiency Comparison 3 of Engineered CRISPR/Cas12f1 System Having U-Rich Tail

Experiments were conducted to evaluate the indel efficiency of an engineered CRISPR/Cas12f1 system having a U-rich tail sequence, using the DYtarget2, DYtarget10, and Intergenic-22 as a target nucleic acid. The protospacer sequence of the target nucleic acid and each PAM sequence are disclosed in Table 10.

TABLE 10
Target nucleic acid sequences used in Experimental Example 4

| Target | | |
|---|---|---|
| nucleic | Protospacer sequence (5′ to 3′) | |
| acid | of target nucleic acid | PAM |
| DYtarget2 | CACACACACAGTGGGCTACC (SEQ ID NO: 63) | TTTG |
| DYtarget10 | CATCCCCAGGACACACACAC (SEQ ID NO: 65) | TTTG |
| Intergenic-22 | AGAACACATACCCCTGGGCC (SEQ ID NO: 67) | TTTA |

1) A plasmid vector including a human codon-optimized Cas12f1 sequence was prepared according to Experimental Example 1-2.

2) According to Experimental Examples 1-2 and 1-4, plasmid vectors and amplicons capable of expressing a tracrRNA, and plasmid vectors and amplicons capable of expressing a (engineered) crRNA with a wild-type or U-rich tail sequence were prepared. DNA sequences encoding the tracrRNA and crRNA, and primer sequences used to prepare plasmid vectors and amplicons are shown in Tables 11 to 13.

TABLE 11
DNA sequence encoding tracrRNA of
Experimental Example 4

| Amplicon sequence encoding | SEQ ID |
|---|---|
| tracrRNA (5′ to 3′) | NO |
| CTTCACTGATAAAGTGGAGAACCGCTTCACCAAAA | 61 |
| GCTGTCCCTTAGGGGATTAGAACTTGAGTGAAGGT | |
| GGGCTGCTTGCATCAGCCTAATGTCGAGAAGTGCT | |
| TTCTTCGGAAAGTAACCCTCGAAACAAATTCATTT | |
| TTCCTCTCCAATTCTGCACAA | |

In this regard, the amplicon sequence or plasmid vector sequence capable of expressing the tracrRNA is an amplicon sequence or plasmid vector sequence in which a U6 promoter is operably linked to the DNA sequence encoding the tracrRNA.

TABLE 12
DNA sequences encoding crRNA of Experimental Example 4

| | Target | crRNA common | Protospacer | U-rich tail | SEQ |
|---|---|---|---|---|---|
| | nucleic | sequence | sequence | sequence | ID |
| Label | acid | (5′ to 3′) | (5′ to 3′) | (3′ to 5′) | NO |
| Canonical | DYtarget2 | GTTGCAGAA | CACACACACAGTGGGC | | 149 |
| crRNA | (Target 1) | CCCGAATAGA | TACC | | |
| | | CGAATGAAG | | | |
| gRNA (MS1) | | GAATGCAAC | CACACACACAGTGGGC | TTTTATTTTTT | 150 |
| | | (SEQ ID | TACC | | |
| | | NO: 255) | | | |
| Canonical | DYtarget10 | | CATCCCCAGGACACAC | | 163 |
| crRNA | (Target 2) | | ACAC | | |
| gRNA (MS1) | | | CATCCCCAGGACACAC | TTTTATTTTTT | 164 |
| | | | ACAC | | |
| Canonical | Intergenic-22 | | AGAACACATACCCCTG | | 176 |
| crRNA | (Target 3) | | GGCC | | |
| gRNA (MS1) | | | AGAACACATACCCCTG | TTTTATTTTTT | 177 |
| | | | GGCC | | |

In the above table, for the DNA sequence encoding crRNA for each target nucleic acid, a crRNA common sequence—a protospacer sequence—a U-rich tail sequence are linked in the 5′-to-3′ direction, and a crRNA repeat sequence in the crRNA common sequence is the sequence of SEQ ID NO: 58. The amplicon sequence or plasmid sequence capable of expressing the (engineered) crRNA is an amplicon sequence or plasmid sequence in which a U6 promoter is operably linked to the DNA sequence encoding the crRNA.

TABLE 13
Primer sequences used in Experimental Example 4

| | Target | Forward | | SEQ |
|---|---|---|---|---|
| | nucleic | primer | Reverse primer | ID |
| Label | acid | (5′ to 3′) | (5′ to 3′) | NO |
| tracrRNA | - | GAGGGCC | TTGTGCAGAATTGGAGAGGA | 236 |
| | | TATTTCCC | | |
| Canonical | DYtarget2 | ATGATT | GGTAGCCCACTGTGTGTGTGGTTGCATTCCT | 207 |
| crRNA | | (SEQ ID | TCATTCGTCTATTC | |
| | | NO: 183) | | |
| gRNA | DYtarget2 | | aaaaaataaaaGGTAGCCCACTGTGTGTGTGGTTG | 208 |
| (MS1) | | | CATTCCTTCATTCGTCTATTC | |
| Canonical | DYtarget10 | | GTGTGTGTGTCCTGGGGATGGTTGCATTCCT | 221 |
| crRNA | | | TCATTCGTCTATTC | |
| gRNA | DYtarget10 | | aaaaataaaaGTGTGTGTGTCCTGGGGATGGTTGC | 222 |
| (MS1) | | | ATTCCTTCATTCGTCaTATTC | |
| Canonical | Intergenic- | | GGCCCAGGGGTATGTGTTCTGTTGCATTCCT | 234 |
| crRNA | 22 | | | |
| gRNA | Intergenic- | | aaaaaataaaaGGCCCAGGGGTATGTGTTCTGTTG | 235 |
| (MS1) | 22 | | CATTCCT | |

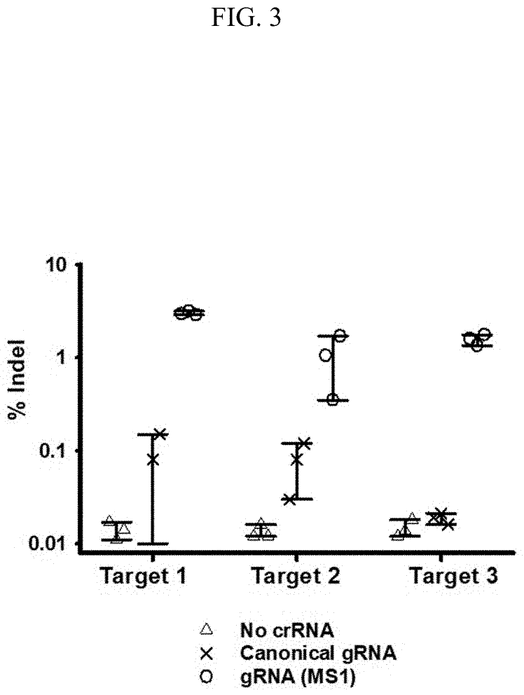

3) HEK-293T cells cultured according to Experimental Example 1-8 were transfected with a plasmid vector including the Cas12f1 sequence, a plasmid vector or amplicon of the tracrRNA, and a plasmid vector or amplicon of the crRNA by the method disclosed in Experimental Example 1-9. In this regard, the one into which crRNA was not injected was used as a control (No crRNA data in FIG. 3).

4) The indel efficiencies for the target nucleic acids of each example were analyzed by the methods disclosed in Experimental Examples 1-12 to 1-14. The indel efficiency analysis results are shown in FIG. 3.

As disclosed in FIG. 3, the CRISPR/Cas12f1 system including an engineered guide RNA with a U-rich tail sequence linked to 3′-terminus showed higher indel efficiency than a the wild-type.

### Experimental Example 5 Indel Efficiency Comparison 4 of Engineered CRISPR/Cas12f1 System Having U-Rich Tail

Experiments are conducted to evaluate the indel efficiency of an engineered CRISPR/Cas12f1 system having a U-rich tail sequence, using various target sequences as a target nucleic acid.

Experiments are conducted on the target nucleic acid disclosed in Experimental Example 3 or 4, and the experiments follow the following method instead of transfecting HEK-293 T cells with a plasmid vector including a Cas12f1 sequence and an amplicon of a single guide RNA.

1) A single guide RNA having the sgRNA sequence disclosed in Experimental Examples 3 and 4 is prepared according to Experimental Example 1-5.

2) A Cas12f1 protein is prepared according to Experimental Example 1-6.

3) RNPs are prepared from the resulting products of 1) and 2) according to Experimental Example 1-7.

4) The HEK-293 T cells prepared according to Experimental Example 1-8 are transfected with the RNPs according to Experimental Example 1-10.

5) The following indel efficiency analysis method is as described in Experimental Example 2 or 3.

### Experimental Example 6 Indel Efficiency Comparison 5 of Engineered CRISPR/Cas12f1 System Having U-Rich Tail

Experiments are conducted to evaluate the indel efficiency of an engineered CRISPR/Cas12f1 system having a U-rich tail sequence, using various target genes as a target nucleic acid.

Experiments are conducted on the target nucleic acid disclosed in Experimental Example 3 or 4, and the experiments follow the following method instead of transfecting HEK-293 T cells with a plasmid vector including a Cas12f1 sequence and an amplicon of a single guide RNA.

1) Viral vectors having the sgRNA sequence disclosed in Experimental Examples 3 and 4 and a sequence encoding a Cas12f1 protein are prepared according to Experimental Example 1-3.

4) The HEK-293 T cells prepared according to Experimental Example 1-8 are transfected with the viral vector according to Experimental Example 1-11.

5) The following indel efficiency analysis method is as described in Experimental Example 2 or 3.

### Experimental Example 7 Indel Efficiency Comparison 1 According to Structure of U-Rich Tail

Experiments were conducted to evaluate the indel efficiency of an engineered CRISPR/Cas12f1 system according to the structure of the U-rich tail sequence, using the DYtarget2 and DYtarget10 as a target nucleic acid. The protospacer sequence of the target nucleic acid and each PAM sequence are disclosed in Table 14.

TABLE 14
Target nucleic acid sequences used in Experimental Example 7

| Target nucleic | Protospacer sequence (5′ to 3′) | |
|---|---|---|
| acid | of target nucleic acid | PAM |
| DYtarget2 | CACACACACAGTGGGCTACC (SEQ ID NO: 63) | TTTG |
| DYtarget10 | CATCCCCAGGACACACACAC (SEQ ID NO: 65) | TTTG |

1) A plasmid vector including a human codon-optimized Cas12f1 sequence was prepared according to Experimental Example 1-2.

2) An amplicon capable of expressing a wild-type, or (an engineered) single guide RNA having a U-rich tail sequence was prepared according to Experimental Example 1-4. DNA sequences encoding the single guide RNA and primer sequences used to prepare an amplicon are shown in Tables 15 and 16.

TABLE 15
DNA sequences encoding sgRNA of Experimental Example 7

| | Target | sgRNA common | Protospacer | U-rich tail | |
|---|---|---|---|---|---|
| | nucleic | sequence | sequence | sequence | SEQ |
| Label | acid | (5′ to 3′) | (5′ to 3′) | (3′ to 5′) | ID NO |
| Ux | DYtarget2 | CTTCACTGAT | CACACACACAGTGGGC | | 129 |
| | | AAAGTGGAG | TACC | | |
| U6 | | AACCGCTTC | | TTTTTT | 135 |
| | | ACCAAAAGC | | | |
| U4AU6 | | TGTCCCTTAG | | TTTTATT | 142 |
| | | GGGATTAGA | | TTTT | |
| | | ACTTGAGTG | | | |
| U3AU6 | | AAGGTGGGC | | TTTATTT | 147 |
| | | TGCTTGCATC | | TTT | |
| | | AGCCTAATGT | | | |
| U3AU3AU6 | | CGAGAAGTG | | TTTATTT | 148 |
| | | CTTTCTTCGG | | ATTTTTT | |
| | | AAAGTAACC | | | |
| Ux | DYtarget10 | CTCGAAACA | CATCCCCAGGACACAC | | 158 |
| | | AATTCATTTT | ACAC | | |
| U6 | | TCCTCTCCAA | | TTTTTT | 160 |
| | | TTCTGCACAA | | | |
| U4AU6 | | gaaaGTTGCAG | | TTTTATT | 159 |
| | | AACCCGAATA | | TTTT | |
| | | GACGAATGA | | | |
| U3AU6 | | AGGAATGCA | | TTTATTT | 161 |
| | | AC (SEQ ID | | TTT | |
| | | NO: 126) | | | |
| U3AU3AU6 | | | | TTTATTT | 162 |
| | | | | ATTTTTT | |

In the above table, for the DNA sequence encoding sgRNA for each target nucleic acid, an sgRNA common sequence—a protospacer sequence—a U-rich tail sequence are linked in the 5′-to-3′ direction, and a scaffold sequence in the sgRNA common sequence is a sequence portion of SEQ ID NO: 254. The amplicon sequence capable of expressing the (engineered) single guide RNA is an amplicon sequence in which a U6 promoter is operably linked to the DNA sequence encoding the sgRNA.

TABLE 16
Primer sequences used in Experimental Example 7

| | Target | Forward | | SEQ |
|---|---|---|---|---|
| | nucleic | primer | | ID |
| Label | acid | (5′ to 3′) | Reverse primer (5′ to 3′) | NO |
| Ux | DYtarget2 | GAGGGCC | GGTAGCCCACTGTGTGTGTGGTTGCATTCCT | 187 |
| | | TATTTCCC | TCATTCGTCTATTC | |
| | | ATGATT | | |
| U6 | | (SEQ ID | aaaaaaGGTAGCCCACTGTGTGTGTGGTTGCAT | 193 |
| | | NO: 183) | TCCTTCATTCGTCTATTC | |
| U4AU6 | | | aaaaaataaaaGGTAGCCCACTGTGTGTGTGGTTG | 200 |
| | | | CATTCCTTCATTCGTCTATTC | |
| U3AU6 | | | aaaaaataaaGGTAGCCCACTGTGTGTGTGGTTG | 205 |
| | | | CATTCCTTCATTCGTCTATTC | |
| U3AU3AU6 | | | aaaaaataaataaaGGTAGCCCACTGTGTGTGTGGT | 206 |
| | | | TGCATTCCTTCATTCGTCTATTC | |
| Ux | DYtarget10 | | GTGTGTGTGTCCTGGGGATGGTTGCATTCCT | 216 |
| | | | TCATTCGTCTATTC | |
| U6 | | | aaaaaaGTGTGTGTGTCCTGGGGATGGTTGCAT | 218 |
| | | | TCCTTCATTCGTCTATTC | |
| U4AU6 | | | aaaaaataaaaGTGTGTGTGTCCTGGGGATGGTTG | 217 |
| | | | CATTCCTTCATTCGTCTATTC | |
| U3AU6 | | | aaaaaataaaGTGTGTGTGTCCTGGGGATGGTTGC | 219 |
| | | | ATTCCTTCATTCGTCTATTC | |
| U3AU3AU6 | | | aaaaaataaataaaGTGTGTGTGTCCTGGGGATGGT | 220 |
| | | | TGCATTCCTTCATTCGTCTATTC | |

3) HEK-293T cells cultured according to Experimental Example 1-8 were transfected with a plasmid vector including the Cas12f1 sequence and an amplicon of the single guide RNA by the method disclosed in Experimental Example 1-9.

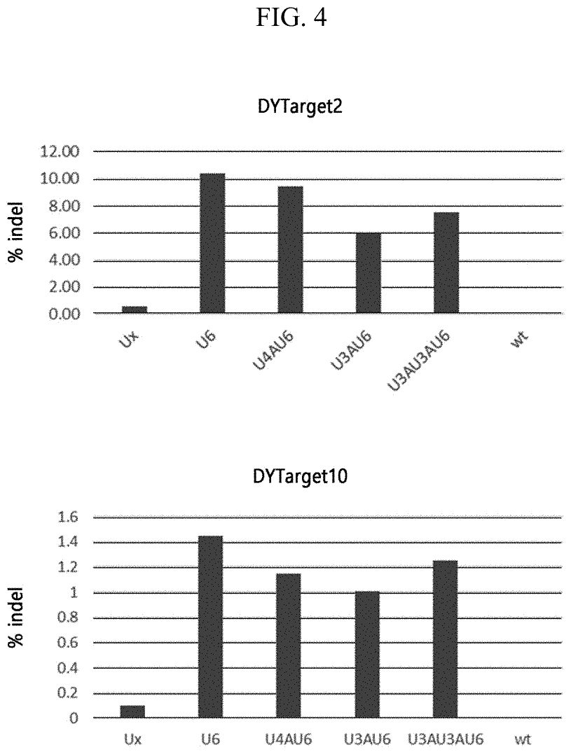

4) The indel efficiencies for the target nucleic acids of each example were analyzed by the methods disclosed in Experimental Examples 1-12 to 1-14. The indel efficiency analysis results are shown in FIG. 4. In this regard, HEK-293T cells that had not been subjected to any treatment were used as a control (see the WT data in FIG. 4).

### Experimental Example 8 Indel Efficiency Comparison 2 According to Structure of U-Rich Tail

Experiments were conducted to evaluate the indel efficiency of an engineered CRISPR/Cas12f1 system according to the structure of the U-rich tail sequence, using the DYTarget2 as a target nucleic acid. The protospacer sequence of the target nucleic acid and the PAM sequence are shown as “DYTarget2” in Table 14 of Experimental Example 7.

1) A plasmid vector including a human codon-optimized Cas12f1 sequence was prepared according to Experimental Example 1-2.

2) An amplicon capable of expressing a wild-type, or (an engineered) single guide RNA having a U-rich tail sequence was prepared according to Experimental Example 1-4. DNA sequences encoding the single guide RNA and primer sequences used to prepare an amplicon are shown in Tables 17 and 18.

TABLE 17
DNA sequences encoding sgRNA of Experimental Example 8

| | Target | sgRNA common | Protospacer | U-rich tail | SEQ |
|---|---|---|---|---|---|
| | nucleic | sequence | sequence | sequence | ID |
| Label | acid | (5′ to 3′) | (5′ to 3′) | (3′ to 5′) | NO |
| None | DYtarget2 | CTTCACTGAT | CACACACACAGTGGGC | | 129 |
| T | | AAAGTGGAG | TACC | T | 130 |
| T2 | | AACCGCTTC | | TT | 131 |
| T3 | | ACCAAAAGC | | TTT | 132 |
| T4 | | TGTCCCTTAG | | TTTT | 133 |
| T4A | | GGGATTAGA | | TTTTA | 136 |
| T4AT | | ACTTGAGTG | | TTTTAT | 137 |
| T4AT2 | | AAGGTGGGC | | TTTTATT | 138 |
| T4AT3 | | TGCTTGCATC | | TTTTATT | 139 |
| | | AGCCTAATGT | | T | |
| T4AT4 | | CGAGAAGTG | | TTTTATT | 140 |
| | | CTTTCTTCGG | | TT | |
| T4AT5 | | AAAGTAACC | | TTTTATT | 141 |
| | | CTCGAAACA | | TTT | |
| T4AT6 | | AATTCATTTT | | TTTTATT | 142 |
| | | TCCTCTCCAA | | TTTT | |
| T4AT7 | | TTCTGCACAA | | TTTTATT | 143 |
| | | gaaaGTTGCAG | | TTTTT | |
| T4AT8 | | AACCCGAATA | | TTTTATT | 144 |
| | | GACGAATGA | | TTTTTT | |
| T4AT4AT | | AGGAATGCA | | TTTTATT | 256 |
| | | AC (SEQ ID | | TTAT | |
| T4AT4AT2 | | NO: 126) | | TTTTATT | 257 |
| | | | | TTATT | |

In the above table, for the amplicon sequence encoding sgRNA for each target nucleic acid, an sgRNA common sequence—a protospacer sequence—a U-rich tail sequence are linked in the 5′-to-3′ direction, and a scaffold sequence in the sgRNA common sequence is a sequence portion of SEQ ID NO: 254. The amplicon sequence capable of expressing the (engineered) single guide RNA is an amplicon sequence in which a U6 promoter is operably linked to the DNA sequence encoding the sgRNA.

TABLE 18
Primer sequences used in Experimental Example 8

| | Target | Forward | | |
|---|---|---|---|---|
| | nucleic | primer | Reverse primer | SEQ |
| Label | acid | (5′ to 3′) | (5′ to 3′) | ID NO |
| None | DYtarget2 | GAGGGCC | GGTAGCCCACTGTGTGTGTGGTTGCATTCCT | 187 |
| | | TATTTCCC | TCATTCGTCTATTC | |
| T | | ATGATT | aGGTAGCCCACTGTGTGTGTGGTTGCATTCCT | 188 |
| | | (SEQ ID | TCATTCGTCTATTC | |
| T2 | | NO: 183) | aaGGTAGCCCACTGTGTGTGTGGTTGCATTCC | 189 |
| | | | TTCATTCGTCTATTC | |
| T3 | | | aaaGGTAGCCCACTGTGTGTGTGGTTGCATTC | 190 |
| | | | CTTCATTCGTCTATTC | |
| T4 | | | aaaaGGTAGCCCACTGTGTGTGTGGTTGCATT | 191 |
| | | | CCTTCATTCGTCTATTC | |
| T4A | | | taaaaGGTAGCCCACTGTGTGTGTGGTTGCATT | 194 |
| | | | CCTTCATTCGTCTATTC | |
| T4AT | | | ataaaaGGTAGCCCACTGTGTGTGTGGTTGCAT | 195 |
| | | | TCCTTCATTCGTCTATTC | |
| T4AT2 | | | aataaaaGGTAGCCCACTGTGTGTGTGGTTGCAT | 196 |
| | | | TCCTTCATTCGTCTATTC | |
| T4AT3 | | | aaataaaaGGTAGCCCACTGTGTGTGTGGTTGCA | 197 |
| | | | TTCCTTCATTCGTCTATTC | |
| T4AT4 | | | aaaataaaaGGTAGCCCACTGTGTGTGTGGTTGC | 198 |
| | | | ATTCCTTCATTCGTCTATTC | |
| T4AT5 | | | aaaaataaaaGGTAGCCCACTGTGTGTGTGGTTG | 199 |
| | | | CATTCCTTCATTCGTCTATTC | |
| T4AT6 | | | aaaaaataaaaGGTAGCCCACTGTGTGTGTGGITG | 200 |
| | | | CATTCCTTCATTCGTCTATTC | |
| T4AT7 | | | aaaaaaataaaaGGTAGCCCACTGTGTGTGTGGTT | 201 |
| | | | GCATTCCTTCATTCGTCTATTC | |
| T4AT8 | | | aaaaaaaataaaaGGTAGCCCACTGTGTGTGTGGTT | 202 |
| | | | GCATTCCTTCATTCGTCTATTC | |
| T4AT4AT | | | ataaaataaaaGGTAGCCCACTGTGTGTGTGGTTG | 258 |
| | | | CATTCCTTCATTCGTCTATTC | |
| T4AT4AT2 | | | aataaaataaaaGGTAGCCCACTGTGTGTGTGGIT | 259 |
| | | | GCATTCCTTCATTCGTCTATTC | |

1) HEK-293T cells cultured according to Experimental Example 1-8 were transfected with a plasmid vector including the Cas12f1 sequence and an amplicon of the single guide RNA by the method disclosed in Experimental Example 1-9.

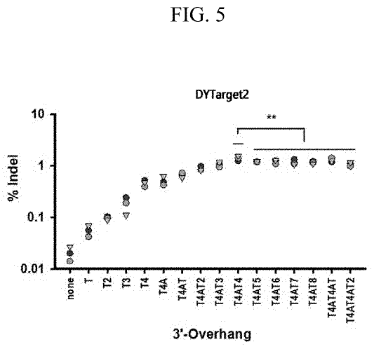

2) The indel efficiencies for the target nucleic acids of each example were analyzed by the methods disclosed in Experimental Examples 1-12 to 1-14. The indel efficiency analysis results are shown in FIG. 5.

### Experimental Example 9 Indel Efficiency Comparison 3 According to Structure of U-Rich Tail

Experiments were conducted to evaluate the indel efficiency of an engineered CRISPR/Cas12f1 system according to the structure of the U-rich tail sequence, using CSMD1 as a target nucleic acid. The protospacer sequence of the target nucleic acid and the PAM sequence are shown in Table 19.

TABLE 19
Target nucleic acid sequence used in
Experimental Example 9

| Target | Protospacer | |
|---|---|---|
| nucleic | sequence (5′ to 3′) | |
| acid | of target nucleic acid | PAM |
| CSMD1 | GAATACCCCCATTCTTCAGG | TTTA |
| | (SEQ ID NO: 260) | |

1) A recombinant Cas12f1 protein was prepared according to Experimental Example 1-6.

2) A wild-type, or (an engineered) single guide RNA having a U-rich tail sequence was synthesized according to Experimental Example 1-5. The sequences of the single guide RNA are shown in the following Table 20.

TABLE 20
sgRNA sequences of Experimental Example 9

| | Target | sgRNA common | | U-rich tail | SEQ |
|---|---|---|---|---|---|
| | nucleic | sequence | Spacer sequence | sequence | ID |
| Label | acid | (5′ to 3′) | (5′ to 3′) | (3′ to 5′) | NO |
| None | CSMD1 | CUUCACUGAUA | GAAUACCCCC | | 261 |
| U | | AAGUGGAGAAC | AUUCUUCAGG | U | 262 |
| U2 | | CGCUUCACCAA | | UU | 263 |
| U3 | | AAGCUGUCCCU | | UUU | 264 |
| U4 | | UAGGGGAUUAG | | UUUU | 265 |
| U5 | | AACUUGAGUGA | | UUUUU | 266 |
| U6 | | AGGUGGGCUGC | | UUUUUU | 267 |
| U4A | | UUGCAUCAGCC | | UUUUA | 268 |
| U4AU | | UAAUGUCGAGA | | UUUUAU | 269 |
| U4AU2 | | AGUGCUUUCUU | | UUUUAUU | 270 |
| U4AU3 | | CGGAAAGUAAC | | UUUUAUUU | 271 |
| U4AU4 | | CCUCGAAACAA | | UUUUAUUUU | 272 |
| U4AU5 | | AUUCAUUUUUC | | UUUUAUUUUU | 273 |
| U4AU6 | | CUCUCCAAUUC | | UUUUAUUUUUU | 274 |
| U4AU7 | | UGCACAAgaaaGU | | UUUUAUUUUUUU | 275 |
| U4AU8 | | UGCAGAACCCG | | UUUUAUUUUUUU | 276 |
| | | AAUAGACGAAU | | U | |
| U4AU9 | | GAAGGAAUGCA | | UUUUAUUUUUUU | 277 |
| | | AC (SEQ ID NO: | | UU | |
| U4AU10 | | 74) | | UUUUAUUUUUUU | 278 |
| | | | | UUU | |

In the above table, for the sgRNA sequence for each target nucleic acid, an sgRNA common sequence—a spacer sequence—a U-rich tail sequence are linked in the 5′-to-3′ direction, and a scaffold sequence in the sgRNA common sequence is a sequence portion of SEQ ID NO: 253.

3) RNPs were prepared according to Experimental Example 1-7 using the recombinant Cas12f1 and the single guide RNA, and the HEK293 T cells cultured according to Experimental Example 1-8 were transfected with the RNPs by the method disclosed in Experimental Example 1-7.

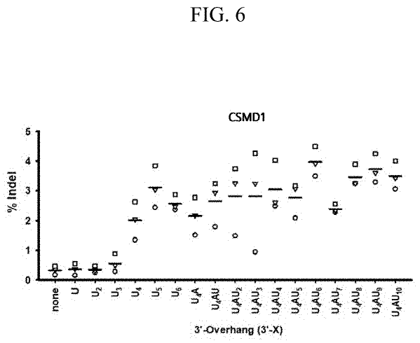

2) The indel efficiencies for the target nucleic acids of each example were analyzed by the methods disclosed in Experimental Examples 1-12 to 1-13. The indel efficiency analysis results are shown in FIG. 6.

As a result of the experiments, it could be seen that that the engineered guide RNA having a U-rich tail sequence linked to 3′-terminus showed a remarkably higher indel efficiency than the wild-type guide RNA having no U-rich tail sequence.

In particular, it could be seen that the engineered guide RNA with a sequence with 10 or more linked uridines showed an excellent indel efficiency compared to the wild-type guide RNA.

### Experimental Example 10 Indel Efficiency Comparison 3 According to Structure of U-Rich Tail

Experiments were conducted to evaluate the indel efficiency of an engineered CRISPR/Cas12f1 system according to the structure of a modified continuous uridine sequence among the U-rich tail sequences, using the DYtarget2 as a target nucleic acid. The protospacer sequence of the target nucleic acid and the PAM sequence are shown as “DYTarget2” in Table 14 of Experimental Example 7.

1) A plasmid vector including a human codon-optimized Cas12f1 sequence was prepared according to Experimental Example 1-2.

2) An amplicon capable of expressing a wild-type, or (an engineered) single guide RNA having a U-rich tail sequence was prepared according to Experimental Example 1-4. DNA sequences encoding the single guide RNA and primer sequences used to prepare an amplicon are shown in Tables 21 and 22.

TABLE 21
DNA sequences encoding sgRNA of Experimental Example 10

| | Target | sgRNA common | | U-rich tail | SEQ |
|---|---|---|---|---|---|
| | nucleic | sequence | Protospacer sequence | sequence | ID |
| Label | acid | (5′ to 3′) | (5′ to 3′) | (3′ to 5′) | NO |
| None | DYtarget2 | CTTCACTGAT | CACACACACAGTGGGC | — | |
| T4AT6 | | AAAGTGGAG | TACC | TTTTATT | |
| | | AACCGCTTC | | TTTT | |
| T4GT6 | | ACCAAAAGC | | TTTTGTT | |
| | | TGTCCCTTAG | | TTTT | |
| | | GGGATTAGA | | | |
| | | ACTTGAGTG | | | |
| | | AAGGTGGGC | | | |
| | | TGCTTGCATC | | | |
| | | AGCCTAATGT | | | |
| | | CGAGAAGTG | | | |
| | | CTTTCTTCGG | | | |
| | | AAAGTAACC | | | |
| | | CTCGAAACA | | | |
| | | AATTCATTTT | | | |
| | | TCCTCTCCAA | | | |
| | | TTCTGCACAA | | | |
| | | gaaaGTTGCAG | | | |
| | | AACCCGAATA | | | |
| | | GACGAATGA | | | |
| | | AGGAATGCA | | | |
| | | AC (SEQ ID | | | |
| | | NO: 126) | | | |

In the above table, for the DNA sequence encoding sgRNA for each target nucleic acid, an sgRNA common sequence—a protospacer sequence—a U-rich tail sequence are linked in the 5′-to-3′ direction, and a scaffold sequence in the sgRNA common sequence is a sequence portion of SEQ ID NO: 254. The amplicon sequence capable of expressing the (engineered) single guide RNA is an amplicon sequence in which a U6 promoter is operably linked to the DNA sequence encoding the sgRNA.

TABLE 22
Primer sequences used in Experimental Example 10

| | Target | Forward | | |
|---|---|---|---|---|
| | nucleic | primer | Reverse primer | SEQ ID |
| Label | acid | (5′ to 3′) | (5′ to 3′) | NO |
| None | DYtarget2 | GAGGGCC | GGTAGCCCACTGTGTGTGTGGTTGCATTCCT | |
| | | TATTTCCC | TCATTCGTCTATTC | |
| T4AT6 | | ATGATT | aaaaaataaaaGGTAGCCCACTGTGTGTGTGGTTG | |
| | | (SEQ ID NO: | CATTCCTTCATTCGTCTATTC | |
| T4GT6 | | 183) | aaaaaacaaaaGGTAGCCCACTGTGTGTGTGGTTG | |
| | | | CATTCCTTCATTCGTCTATTC | |

3) HEK-293T cells cultured according to Experimental Example 1-8 were transfected with a plasmid vector including the nucleic acid sequence encoding the Cas12f1 sequence and an amplicon of the single guide RNA by the method disclosed in Experimental Example 1-9.

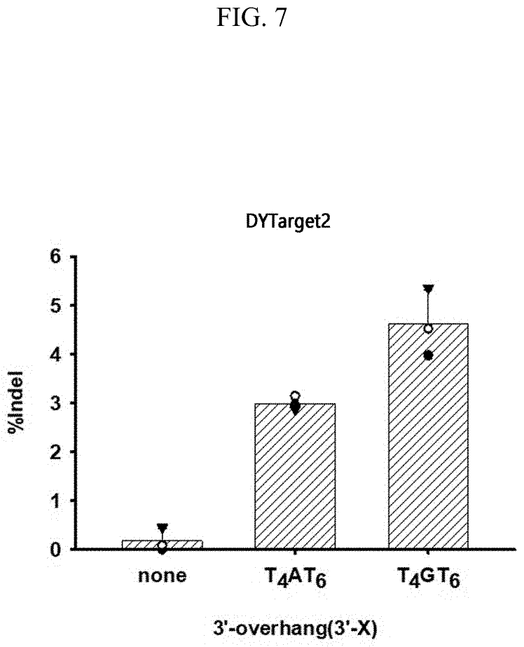

4) The indel efficiencies for the target nucleic acids of each example were analyzed by the methods disclosed in Experimental Examples 1-12 to 1-14. The indel efficiency analysis results are shown in FIG. 7.

As a result of the experiment, it was shown that when the engineered guide RNA has a modified continuous uridine sequence structure which can be represented by (UₐV)ₙU_{b} (in this regard, V is one of cytidine (C), guanosine (G), or adenosine (A)), the indel efficiency was remarkably higher in eukaryotic cells than the wild-type guide RNA having no U-rich tail sequence.

### Experimental Example 11 Comparison of Indel Efficiency According to Length of Spacer Sequence

In order to compare the indel efficiency according to the length of the spacer sequence, the DYtarget2 and DYtarget10 were used as target nucleic acids, the U-rich tail sequence was fixed as U4AU6, and then the experiment was performed by changing the length of the spacer sequence. The protospacer sequences of the target nucleic acid and each PAM sequence are disclosed in Table 23.

TABLE 23
Target nucleic acid sequences used in
Experimental Example 11

| | Protospacer sequence | |
|---|---|---|
| Target | (5′ to 3′) of target | |
| nucleic acid | nucleic acid | PAM |
| DYtarget2 | CACACACACAGTGGGCTACC | TTTG |
| | (SEQ ID NO: 63) | |
| DYtarget10 | CATCCCCAGGACACACACAC | TTTG |
| | (SEQ ID NO: 65) | |

1) A plasmid vector including a nucleic acid sequence encoding a human codon-optimized Cas12f1 sequence was prepared according to Experimental Example 1-2.

2) An amplicon capable of expressing an engineered single guide RNA having a length of each different spacer sequence was prepared according to Experimental Example 1-4. DNA sequences encoding the single guide RNA, and primer sequences used to prepare an amplicon are shown in Tables 24 and 25.

TABLE 24
DNA sequences encoding sgRNA used in Experimental Example 11

| | Target | sgRNA common | sequence | U-rich tail | SEQ |
|---|---|---|---|---|---|
| | nucleic | sequence | Protospacer | sequence | ID |
| Label | acid | (5′ to 3′) | (5′ to 3′) | (3′ to 5′) | NO |
| 18mer | DYtarget2 | CTTCACTGAT | CACACACACAGTGGGC | TTTTATT | 151 |
| | | AAAGTGGAG | TA | TTTT | |
| 19mer | | AACCGCTTC | CACACACACAGTGGGC | | 152 |
| | | ACCAAAAGC | TAC | | |
| 20mer | | TGTCCCTTAG | CACACACACAGTGGGC | | 142 |
| | | GGGATTAGA | TACC | | |
| 21mer | | ACTTGAGTG | CACACACACAGTGGGC | | 153 |
| | | AAGGTGGGC | TACCA | | |
| 25mer | | TGCTTGCATC | CACACACACAGTGGGC | | 154 |
| | | AGCCTAATGT | TACCATTTA | | |
| 30mer | | CGAGAAGTG | CACACACACAGTGGGC | | 155 |
| | | CTTTCTTCGG | TACCATTTAAGGAG | | |
| 17mer | DYtarget10 | AAAGTAACC | CATCCCCAGGACACAC | | 165 |
| | | CTCGAAACA | A | | |
| 18mer | | AATTCATTTT | CATCCCCAGGACACAC | | 166 |
| | | TCCTCTCCAA | AC | | |
| 19mer | | TTCTGCACAA | CATCCCCAGGACACAC | | 167 |
| | | gaaaGTTGCAG | ACA | | |
| 20mer | | AACCCGAATA | CATCCCCAGGACACAC | | 159 |
| | | GACGAATGA | ACAC | | |
| 21mer | | AGGAATGCA | CATCCCCAGGACACAC | | 168 |
| | | AC (SEQ ID | ACACA | | |
| 25mer | | NO: 126) | CATCCCCAGGACACAC | | 169 |
| | | | ACACATTTC | | |
| 30mer | | | CATCCCCAGGACACAC | | 170 |
| | | | ACACATTTCAAAAG | | |

In the above table, for the DNA sequence encoding sgRNA for each target nucleic acid, an sgRNA common sequence—a protospacer sequence—a U-rich tail sequence are linked in the 5′-to-3′ direction, and a scaffold sequence in the sgRNA common sequence is a sequence portion of SEQ ID NO: 254. The amplicon sequence capable of expressing the (engineered) single guide RNA is an amplicon sequence in which a U6 promoter is operably linked to the DNA sequence encoding the sgRNA.

TABLE 25
Primer sequences used in Experimental Example 11

| | Target | Forward | | SEQ |
|---|---|---|---|---|
| | nucleic | primer | Reverse primer | ID |
| Label | acid | (5′ to 3′) | (5′ to 3′) | NO |
| 18mer | DYtarget2 | GAGGGCC | aaaaaataaaaTAGCCCACTGTGTGTGTGGTTGCA | 209 |
| | | TATTTCCC | TTCCT | |
| 19mer | | ATGATT | aaaaaataaaaGTAGCCCACTGTGTGTGTGGTTGC | 210 |
| | | (SEQ ID NO: | ATTCCT | |
| 20mer | | 183) | aaaaaataaaaGGTAGCCCACTGTGTGTGTGGTTG | 200 |
| | | | CATTCCT | |
| 21mer | | | aaaaaataaaaTGGTAGCCCACTGTGTGTGTGGTT | 211 |
| | | | GCATTCCT | |
| 25mer | | | aaaaaataaaaTAAATGGTAGCCCACTGTGTGTGT | 212 |
| | | | GGTTGCATTCCT | |
| 30mer | | | aaaaaataaaaCTCCTTAAATGGTAGCCCACTGTG | 213 |
| | | | TGTGTGGTTGCATTCCT | |
| 17mer | DYtarget10 | | aaaaaataaaaTGTGTGTCCTGGGGATGGTTGCAT | 223 |
| | | | TCCT | |
| 18mer | | | aaaaaataaaaGTGTGTGTCCTGGGGATGGTTGCA | 224 |
| | | | TTCCT | |
| 19mer | | | aaaaaataaaaTGTGTGTGTCCTGGGGATGGTTGC | 225 |
| | | | ATTCCT | |
| 20mer | | | aaaaaataaaaGTGTGTGTGTCCTGGGGATGGTTG | 217 |
| | | | CATTCCT | |
| 21mer | | | aaaaaataaaaTGTGTGTGTGTCCTGGGGATGGIT | 226 |
| | | | GCATTCCT | |
| 25mer | | | aaaaaataaaaGAAATGTGTGTGTGTCCTGGGGAT | 227 |
| | | | GGTTGCATTCCT | |
| 30mer | | | aaaaaataaaaCTTTTGAAATGTGTGTGTGTCCTG | 228 |
| | | | GGGATGGTTGCATTCCT | |

3) HEK-293T cells cultured according to Experimental Example 1-8 were transfected with a plasmid vector including the Cas12f1 sequence and an amplicon of the single guide RNA by the method disclosed in Experimental Example 1-9.

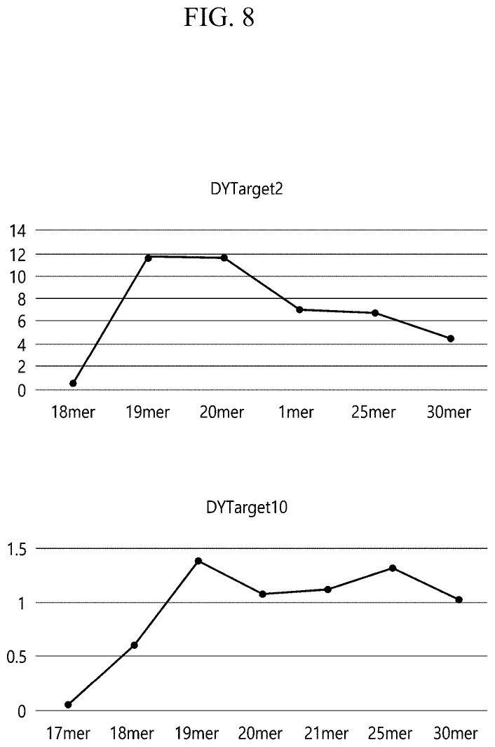

4) The indel efficiencies for the target nucleic acids of each example were analyzed by the methods disclosed in Experimental Examples 1-12 to 1-14. The indel efficiency analysis results are shown in FIG. 8.

### Experimental Example 12 Confirmation of Intracellular Gene Cleavage Activity of Type V CRISPR/Cas System Having U-Rich Tail

In order to confirm whether the gene cleavage activity is enhanced when the U-rich tail sequence is introduced into the Type V CRISPR/Cas system other than the engineered CRISPR/Cas14a system used in the experimental example, the following experiment was conducted on the CRISPR/SpCas12f1 system and the CRISPR/AsCas12f1 system (Karvelis et al., Nucleic Acids Research, Vol. 48, No. 95017 (2020)), which are classified as CRISPR/Cas12f1 systems.

### Experimental Example 12-1. Confirmation of Intracellular Expression of SpCas12f1 and AsCas12f1 Proteins

In order to confirm whether the SpCas12f1 and AsCas12f1 proteins are properly expressed in eukaryotic cells when a vector encoding the SpCas12f1 and AsCas12f1 proteins is introduced into eukaryotic cells, the expression of SpCas12f1 and AsCas12f1 proteins was confirmed by the method disclosed in Experimental Example 1-13. In this regard, amino acid sequences of the SpCas12f1 and AsCas12f1 proteins and human codon-optimized nucleic acid sequences encoding the amino acid sequences are shown in Table 26.

TABLE 26
Sequence information of SpCas12f1 and AsCas12f1 proteins

| | | SEQ |
|---|---|---|
| | | ID |
| Subject | Sequence information | NO |
| SpCas12f1 | MGESVKAIKLKILDMFLDPECTKQDDNWRKDLSTMSRFCAEAGNMCLR | 282 |
| (Amino | DLYNYFSMPKEDRISSKDLYNAMYHKTKLLHPELPGKVANQIVNHAKDV | |
| acid) | WKRNAKLIYRNQISMPTYKITTAPIRLQNNIYKLIKNKNKYIIDVQLYSKEY | |
| | SKDSGKGTHRYFLVAVRDSSTRMIFDRIMSKDHIDSSKSYTQGQLQIKKDH | |
| | QGKWYCIIPYTFPTHETVLDPDKVMGVDLGVAKAVYWAFNSSYKRGCID | |
| | GGEIEHFRKMIRARRVSIQNQIKHSGDARKGHGRKRALKPIETLSEKEKNF | |
| | RDTINHRYANRIVEAAIKQGCGTIQIENLEGIADTTGSKFLKNWPYYDLQT | |
| | KIVNKAKEHGITVVAINPQYTSQRCSMCGYIEKTNRSSQAVFECKQCGYG | |
| | SRTICINCRHVQVSGDVCEECGGIVKKENVNADYNAAKNISTPYIDQIIME | |
| | KCLELGIPYRSITCKECGHIQASGNTCEVCGSTNILKPKKIRKAK | |
| SpCas12f1 | ATGGGCGAGAGCGTTAAAGCAATCAAACTGAAGATCCTGGACATGTTC | 284 |
| (Human | CTGGACCCGGAGTGCACTAAACAGGACGATAACTGGCGTAAAGACCTG | |
| codon- | TCTACCATGTCTCGTTTCTGCGCAGAGGCGGGTAACATGTGCCTGCGCG | |
| optimized | ACCTGTACAATTACTTTTCTATGCCGAAAGAAGACCGTATTAGCTCCAA | |
| nucleic | GGATCTGTACAACGCCATGTACCACAAAACTAAACTGCTGCACCCGGA | |
| acid) | ACTGCCGGGTAAAGTGGCGAACCAGATCGTCAACCACGCTAAAGATGT | |
| | GTGGAAACGCAACGCGAAGCTGATCTATCGTAATCAGATCTCTATGCCG | |
| | ACTTACAAGATCACCACCGCACCGATTCGCCTGCAAAACAACATCTATA | |
| | AACTGATCAAAAACAAGAACAAATATATCATCGATGTCCAGCTGTACAG | |
| | CAAAGAATACTCTAAAGACAGCGGCAAAGGCACCCACCGCTACTTCCT | |
| | GGTTGCAGTGCGCGATTCTAGCACGCGTATGATCTTCGACCGCATCATG | |
| | AGCAAAGACCACATCGATAGCTCCAAATCCTACACCCAGGGTCAGCTG | |
| | CAGATTAAAAAAGACCATCAGGGCAAATGGTACTGTATCATTCCGTACA | |
| | CCTTCCCGACTCATGAGACCGTCCTGGATCCGGACAAAGTTATGGGTGT | |
| | GGACCTGGGCGTGGCTAAAGCAGTGTATTGGGCGTTCAACTCCTCTTAC | |
| | AAACGCGGTTGTATCGATGGTGGTGAAATTGAACACTTCCGCAAAATG | |
| | ATCCGTGCTCGTCGTGTCTCCATTCAGAACCAGATTAAACATTCTGGCG | |
| | ACGCGCGCAAGGGTCATGGTCGTAAACGTGCACTGAAGCCGATTGAAA | |
| | CCCTGTCTGAAAAAGAAAAAAACTTCCGCGATACTATCAACCATCGTTA | |
| | CGCAAACCGTATCGTGGAGGCAGCGATTAAGCAGGGCTGCGGCACCAT | |
| | CCAGATCGAAAATCTGGAGGGTATCGCCGATACTACGGGTTCCAAATTT | |
| | CTGAAAAACTGGCCATACTATGACCTGCAGACCAAAATCGTGAACAAA | |
| | GCGAAGGAACATGGCATCACCGTCGTTGCGATCAACCCGCAGTATACTT | |
| | CTCAACGTTGCTCCATGTGTGGTTACATTGAAAAAACCAACCGTTCCTC | |
| | TCAGGCAGTGTTCGAGTGCAAACAGTGCGGCTATGGTTCCCGTACCATC | |
| | TGCATCAACTGCCGCCACGTTCAGGTAAGCGGTGATGTCTGCGAAGAA | |
| | TGCGGCGGCATCGTTAAAAAAGAAAACGTTAACGCGGACTATAACGCG | |
| | GCGAAAAACATCTCCACCCCATATATCGATCAGATCATCATGGAGAAAT | |
| | GTCTGGAGCTGGGTATCCCGTACCGTAGCATCACTTGCAAAGAATGCGG | |
| | TCATATCCAGGCGTCTGGTAACACTTGTGAGGTGTGCGGTTCCACCAAC | |
| | ATTCTGAAACCGAAAAAAATCCGTAAAGCTAAA | |
| AsCas12f1 | MIKVYRYEIVKPLDLDWKEFGTILRQLQQETRFALNKATQLAWEWMGFS | 283 |
| (amino | SDYKDNHGEYPKSKDILGYTNVHGYAYHTIKTKAYRLNSGNLSQTIKRAT | |
| acid) | DRFKAYQKEILRGDMSIPSYKRDIPLDLIKENISVNRMNHGDYIASLSLLSN | |
| | PAKQEMNVKRKISVIIIVRGAGKTIMDRILSGEYQVSASQIIHDDRKNKWY | |
| | LNISYDFEPQTRVLDLNKIMGIDLGVAVAVYMAFQHTPARYKLEGGEIENF | |
| | RRQVESRRISMLRQGKYAGGARGGHGRDKRIKPIEQLRDKIANFRDTTNH | |
| | RYSRYIVDMAIKEGCGTIQMEDLTNIRDIGSRFLQNWTYYDLQQKIIYKAE | |
| | EAGIKVIKIDPQYTSQRCSECGNIDSGNRIGQAIFKCRACGYEANADYNAA | |
| | RNIAIPNIDKIIAESIK | |
| AsCas12f1 | ATGATCAAGGTTTATCGCTATGAAATTGTGAAACCGCTGGACCTGGACT | 285 |
| (human | GGAAAGAGTTCGGTACTATCCTGCGTCAGCTGCAGCAGGAAACGCGTT | |
| codon- | TCGCACTGAACAAAGCGACGCAGCTGGCTTGGGAATGGATGGGCTTCA | |
| optimized | GCTCTGATTACAAAGACAACCACGGCGAATACCCTAAATCCAAAGATAT | |
| nucleic | CCTGGGTTACACCAACGTGCACGGTTATGCGTACCACACCATCAAAACT | |
| acid) | AAAGCGTACCGCCTGAACTCCGGTAATCTGTCTCAGACCATCAAGCGT | |
| | GCGACCGACCGTTTCAAAGCGTACCAGAAGGAAATCCTGCGCGGTGAC | |
| | ATGAGCATCCCGTCCTATAAGCGTGACATCCCGCTGGATCTGATCAAAG | |
| | AAAACATCAGCGTAAACCGCATGAACCACGGTGACTACATCGCCTCCC | |
| | TGTCTCTGCTGTCTAACCCGGCAAAGCAAGAGATGAACGTTAAACGCA | |
| | AAATCTCCGTTATCATTATCGTTCGTGGCGCAGGCAAAACCATCATGGAT | |
| | CGTATTCTGTCCGGTGAATATCAAGTGTCCGCAAGCCAGATCATCCATG | |
| | ACGATCGTAAAAATAAATGGTATCTGAACATCTCTTATGATTTCGAACCG | |
| | CAGACCCGCGTACTGGACCTGAACAAAATCATGGGTATCGATCTGGGC | |
| | GTTGCAGTAGCGGTGTACATGGCTTTCCAGCACACCCCGGCACGCTAC | |
| | AAACTGGAAGGTGGTGAGATCGAAAACTTCCGTCGTCAAGTGGAATCT | |
| | CGTCGCATCTCTATGCTGCGTCAGGGTAAATATGCAGGTGGTGCGCGTG | |
| | GTGGTCATGGTCGTGACAAACGTATTAAACCAATCGAACAGCTGCGCG | |
| | ATAAAATCGCCAACTTCCGTGATACCACCAACCACCGCTACTCTCGTTA | |
| | TATCGTTGATATGGCAATCAAAGAGGGTTGTGGTACTATCCAGATGGAA | |
| | GACCTGACGAACATCCGCGATATCGGTTCTCGTTTCCTGCAGAACTGGA | |
| | CCTACTACGATCTGCAGCAGAAAATTATCTACAAAGCGGAGGAAGCAG | |
| | GTATCAAAGTCATTAAAATCGATCCGCAGTATACTAGCCAGCGCTGCAG | |
| | CGAATGTGGTAACATTGATAGCGGCAACCGCATTGGTCAGGCAATCTTT | |
| | AAATGCCGCGCTTGTGGCTACGAAGCGAACGCAGACTACAACGCGGCG | |
| | CGTAACATCGCCATCCCGAATATTGACAAAATCATTGCCGAATCTATTAA | |
| | A | |

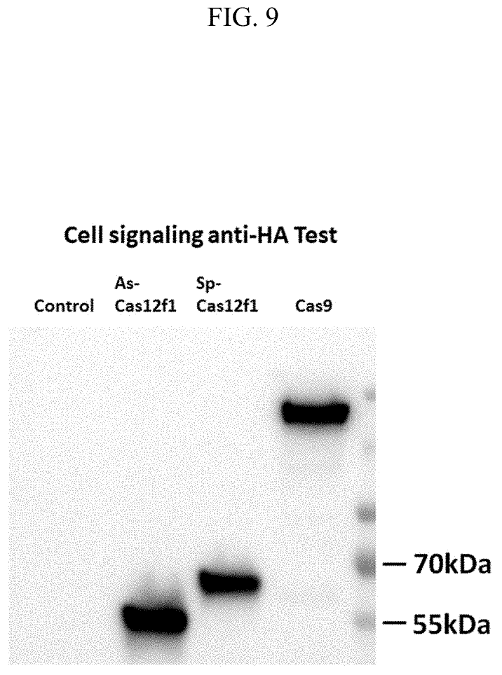

As a result of the experiment, it was confirmed that the SpCas12f1 protein and the AsCas12f1 protein were properly expressed in HEK293T cells (FIG. 9).

### Experimental Example 12-2. Confirmation of Intracellular Gene Cleavage Activity of Engineered CRISPR/SpCas12f1 and CRISPR/AsCas12f1 Systems Having U-Rich Tail

In order to confirm the gene cleavage activity of the engineered CRISPR/SpCas12f1 system and CRISPR/AsCas12f1 system having a U-rich tail sequence, experiments were conducted using various gene sequences as a target nucleic acid. The protospacer sequences of the target nucleic acid and each PAM sequence are disclosed in Table 27.

TABLE 27
Target nucleic acids used in Experimental Example 12

| Target | Protospacer sequence (5′ to 3′) | |
|---|---|---|
| nucleic acid | of target nucleic acid | PAM |
| DYtarget2 | CACACACACAGTGGGCTACC (SEQ ID NO: 63) | TTTG |
| VEGFA-3 | GGGGTGACCGCCGGAGCGCG (SEQ ID NO: 286) | TTTG |

1) Plasmid vector including human codon-optimized sequences encoding SpCas12f1 and AsCas12f1 proteins were prepared according to Experimental Example 1-2.

2) According to Experimental Example 1-2, a plasmid vector expressing tracrRNA, and a plasmid vector expressing a wild-type crRNA or a (engineered) crRNA having a U-rich tail sequence were prepared. DNA sequences encoding the tracrRNA and crRNA, and primer sequences used to prepare plasmid vectors are shown in Tables 28 to 30.

TABLE 28
DNA sequence encoding tracrRNA of Experimental Example 12

| tracrRNA sequence | |
|---|---|
| (5′ to 3′) | SEQ ID NO |
| CTTCACTGATAAAGTGGAGAACCGCTTCACCAAAAGCTGTCCCTTAG | 287 |
| GGGATTAGAACTTGAGTGAAGGTGGGCTGCTTGCATCAGCCTAATGT | |
| CGAGAAGTGCTTTCTTCGGAAAGTAACCCTCGAAACAAATTCATTTT | |
| TCCTCTCCAATTCTGCACAA | |

The CRISPR/SpCas12f1 and CRISPR/AsCas12f1 systems have the same tracrRNA sequence. The plasmid vector sequence which expresses the tracrRNA is a plasmid vector sequence in which a U6 promoter is operably linked to the DNA sequence encoding the tracrRNA.

TABLE 29
DNA sequences encoding crRNA of Experimental Example 12

| | Target | crRNA common | | U-rich tail | SEQ |
|---|---|---|---|---|---|
| | nucleic | sequence | Protospacer sequence | sequence | ID |
| Label | acid | (5′ to 3′) | (5′ to 3′) | (3′ to 5′) | NO |
| SpCas12f1- | DYtargete | GTCGCATCTT | CACACACACAGTGGGC | | 290 |
| DYtarget2(WT) | | GCGTAAGCG | TACC | | |
| SpCas12f1- | | CGTGGATTGA | | TTTTTT | 291 |
| DYtarget2 (U6) | | AAC (SEQ ID | | | |
| SpCas12f1- | | NO: 288) | | TTTTATT | 292 |
| DYtarget2(U4AU6) | | | | TTTT | |
| SpCas12f1- | VEGFA-3 | | GGGGTGACCGCCGGAG | | 293 |
| VEGFA-3(WT) | | | CGCG | | |
| SpCas12f1- | | | | TTTTTT | 294 |
| VEGFA-3(U6) | | | | | |
| SpCas12f1- | | | | TTTTATT | 295 |
| VEGFA-3(U4AU6) | | | | TTTT | |
| AsCas12f1- | DYtarget2 | GTTTGCGAG | CACACACACAGTGGGC | | 296 |
| DYtarget2(WT) | | CTAGCTTGTG | TACC | | |
| AsCas12f1- | | GAGTGTGAA | | TTTTTT | 297 |
| DYtarget2(U6) | | C (SEQ ID NO: | | | |
| AsCas12f1- | | 289) | | TTTTATT | 298 |
| DYtarget2(U4AU6) | | | | TTTT | |
| AsCas12f1- | VEGFA-3 | | GGGGTGACCGCCGGAG | | 299 |
| VEGFA-3(WT) | | | CGCG | | |
| AsCas12f1- | | | | TTTTTT | 300 |
| VEGFA-3(U6) | | | | | |
| AsCas12f1- | | | | TTTTATT | 301 |
| VEGFA-3(U4AU6) | | | | TTTT | |

In the above table, for the DNA sequence encoding crRNA for each target nucleic acid, a crRNA common sequence—a protospacer sequence—a U-rich tail sequence are linked in the 5′-to-3′ direction. The plasmid vector sequence which expresses the crRNA is a plasmid vector sequence in which a U6 promoter is operably linked to the DNA sequence encoding the crRNA.

TABLE 30
Primer sequences used in Experimental Example 12

| | Target | | | SEQ |
|---|---|---|---|---|
| | nucleic | Forward primer | Reverse primer | ID |
| Label | acid | (5′ to 3′) | (5′ to 3′) | NO |
| tracrRNA | — | GAGGGCCTAT | TTGTGCAGAATTGGAG | 302 |
| | | TTCCCATGAT | AGGA | |
| SpCas12f1- | DYtarget2 | T (SEQ ID NO: | GGTAGCCCACTGTGTG | 303 |
| DYtarget2(WT) | | 183) | TGTGgtttcaatcca | |
| SpCas12f1- | | | aaaaaaGGTAGCCCACTG | 304 |
| DYtarget2(U6) | | | TGTGTGTGgtttcaatcca | |
| SpCas12f1- | | | aaaaaataaaaGGTAGCCCA | 305 |
| DYtarget2(U4AU6) | | | CTGTGTGTGTGgtttcaatcc | |
| | | | a | |
| SpCas12f1- | VEGFA-3 | | CGCGCTCCGGCGGTCA | 306 |
| VEGFA-3(WT) | | | CCCCgtttcaatcca | |
| SpCas12f1- | | | aaaaaaCGCGCTCCGGCG | 307 |
| VEGFA-3(U6) | | | GTCACCCCgtttcaatcca | |
| SpCas12f1- | | | aaaaaataaaaCGCGCTCCG | 308 |
| VEGFA-3(U4AU6) | | | GCGGTCACCCCgtttcaatc | |
| | | | ca | |
| AsCas12f1- | DYtarget2 | | GGTAGCCCACTGTGTG | 309 |
| DYtarget2(WT) | | | TGTGgttcacactcc | |
| AsCas12f1- | | | aaaaaaGGTAGCCCACTG | 310 |
| DYtarget2(U6) | | | TGTGTGTGgttcacactcc | |
| AsCas12f1- | | | aaaaaataaaaGGTAGCCCA | |
| DYtarget2(U4AU6) | | | CTGTGTGTGTGgttcacact | 311 |
| | | | cc | |
| AsCas12f1- | VEGFA-3 | | CGCGCTCCGGCGGTCA | 312 |
| VEGFA-3(WT) | | | CCCCgttcacactcc | |
| AsCas12f1- | | | aaaaaaCGCGCTCCGGCG | 313 |
| VEGFA-3(U6) | | | GTCACCCCgttcacactcc | |
| AsCas12f1- | | | aaaaaataaaaCGCGCTCCG | 314 |
| VEGFA-3(U4AU6) | | | GCGGTCACCCCgttcacact | |
| | | | cc | |

3) HEK-293T cells cultured according to Experimental Example 1-8 were transfected with plasmid vectors including nucleic acid sequences encoding the SpCas12f1 protein, the AsCas12f1 protein, a plasmid vector of the tracrRNA, and a plasmid vector of the crRNA by the method disclosed in Experimental Example 1-9.

4) The indel efficiencies for the target nucleic acids of each example were analyzed by the methods disclosed in Experimental Examples 1-12 to 1-14. The indel efficiency analysis results are shown in Table 31.

TABLE 31
Indel efficiency analysis results of
Experimental Example 12

| | Target | Indel |
|---|---|---|
| | nucleic | efficiency |
| Label | acid | (%) |
| SpCas12f1-DYtarget2(WT) | DYtarget2 | 0.00 |
| SpCas12f1 -DYtarget2(U6) | | 0.06 |
| SpCas12f1-DYtarget2(U4AU6) | | 0.07 |
| SpCas12f1-VEGFA-3(WT) | VEGFA-3 | 0.00 |
| SpCas12f1-VEGFA-3(U6) | | 0.00 |
| SpCas12f1-VEGFA-3(U4AU6) | | 0.00 |
| AsCas12f1-DYtarget2(WT) | DYtarget2 | 0.00 |
| AsCas12f1-DYtarget2(U6) | | 0.06 |
| AsCas12f1-DYtarget2(U4AU6) | | 0.03 |
| AsCas12f1-VEGFA-3(WT) | VEGFA-3 | 0.00 |
| AsCas12f1-VEGFA-3(U6) | | 0.01 |
| AsCas12f1-VEGFA-3(U4AU6) | | 0.00 |

As a result of the experiment, no significant indel efficiency was observed in either the engineered CRISPR/SpCas12f1 system into which the U-rich tail sequence has been introduced or the engineered CRISPR/AsCas12f1 system. In conclusion, although it was confirmed that SpCas12f1 and AsCas12f1 proteins are expressed in eukaryotic cells (FIG. 9), no significant indel efficiency has been shown, so that it can be seen that even when a U-rich tail is introduced into the CRISPR/SpCas12f1 system and the CRISPR/AsCas12f1 system, gene cleavage activity is not affected.

### INDUSTRIAL APPLICABILITY

The present disclosure provides a CRISPR/Cas12f1 system which can be used for a gene editing technique, and particularly, an engineered CRISPR/Cas12f1 system of which gene editing efficiency is improved by the introduction of a U-rich tail sequence in crRNA.

When used for gene editing, the engineered CRISPR/Cas12f1 system having the U-rich tail sequence according to the present disclosure exhibits higher gene editing efficiency than a wild-type CRISPR/Cas12f1 system.

1. An engineered CRISPR RNA (crRNA) for a CRISPR/Cas12f1 system capable of editing a nucleic acid including a target sequence, the engineered crRNA comprising:
a spacer sequence which is complementary to the target sequence;
a U-rich tail sequence which is linked to a 3′-terminus of the spacer sequence;
wherein the U-rich tail sequence is represented by the following formula: (UₐN)ₙU_{b}, wherein: N is one of adenosine (A), uridine (U), cytidine (C), and guanosine (G), a is an integer between 1 to 4 inclusive, n is an integer selected from 0, 1, and 2, and b is an integer between 1 to 10 inclusive, and
the sequence of the engineered crRNA includes a crRNA repeat sequence, the spacer sequence, and the U-rich tail sequence, which are linked sequentially in a 5′-to-3′ direction thereof.

2-4. (canceled)

5. An engineered guide RNA for CRISPR/Cas12f1 system capable of editing a nucleic acid including a target sequence, having a sequence comprising:
a tracrRNA sequence;
a crRNA sequence including a CRISPR RNA repeat sequence and a spacer sequence which is complementary to the target sequence;
a U-rich tail sequence which is linked to 3′-terminus of the crRNA sequence wherein the U-rich tail sequence is represented by the formula of claim 1.

6. The engineered guide RNA of claim 5, wherein the engineered guide RNA further comprises a linker sequence, wherein the tracrRNA sequence and the crRNA sequence are linked via the linker sequence.

7. The engineered guide RNA of claim 6, wherein the linker sequence is 5′-gaaa-3′.

8. The engineered guide RNA of claim 5, wherein the U-rich tail sequence is UUUUUU, UUUUAUUUUUU, or UUUUGUUUUUU.

9. (canceled)

10. An engineered CRISPR/Cas12f1 complex capable of editing a nucleic acid including a target sequence, the engineered CRISPR/Cas12f1 complex comprising:
a Cas12f1 protein belonging to a Cas14 family; and
an engineered guide RNA comprising
a scaffold sequence which interacts with the Cas12f1 protein,
a spacer sequence which is complementary to a target sequence,
a U-rich tail sequence, wherein
the U-rich tail sequence is represented by the formula of claim 1.

11. A vector for expressing an engineered CRISPR/Cas12f1 complex capable of editing a nucleic acid including a target sequence in a cell, the vector comprising:
a first sequence including a sequence that encodes a Cas12f1 protein;
a first promoter sequence operably linked to the first sequence;
a second sequence including a sequence that encodes an engineered guide RNA, wherein the engineered guide RNA has a scaffold sequence which interacts with the Cas12f1 protein, a spacer sequence which is complementary to the target sequence, and a U-rich tail sequence which is linked to a 3′-terminus of the spacer sequence, the U-rich tail sequence is represented by the formula of claim 1; and
a second promoter sequence operably linked to the second sequence,
wherein the vector is constructed to express the Cas12f1 protein and the engineered guide RNA, so that the Cas12f1 protein and the engineered guide sequence forms the CRISPR/Cas12f1 complex in the cell, and a nucleic acid is edited by the CRISPR/Cas12f1 complex.

12. (canceled)

13. The vector of claim 11, wherein the vector is a viral vector, a plasmid vector, or a linear PCR amplicon.

14. (canceled)

15. The vector of claim 13, wherein the vector is one or more selected from the group consisting of a retrovirus, a lentivirus, an adenovirus, an adeno-associated virus (AAV), a vaccinia virus, a poxvirus and a herpes simplex virus.

16. (canceled)

17. A vector for expressing an engineered CRISPR/Cas12f1 complex capable of editing a nucleic acid including a first target sequence and a second target sequence, the vector comprising:
a first sequence including a sequence that encodes a Cas12f1 protein;
a first promoter sequence operably linked to the first sequence;
a second sequence including a sequence that encodes a first engineered guide RNA, wherein the first engineered guide RNA has a first scaffold sequence which interacts with the Cas12f1 protein, a first spacer sequence which is complementary to a first target sequence, and a first U-rich tail sequence, wherein the first U-rich tail sequence is represented by the formula of claim 1;
a second promoter sequence operably linked to the second sequence;
a third sequence including a sequence that encodes a second engineered guide RNA, wherein the second engineered guide RNA has a second scaffold sequence which interacts with the Cas12f1 protein, a second spacer sequence which is complementary to a second target sequence, and a second U-rich tail sequence, wherein the second U-rich tail sequence is represented by the formula of claim 1; and a third promoter sequence operably linked to the third sequence.

18. The vector of claim 17, wherein the second promoter sequence and the third promoter sequence are the same promoter sequence.

19. The vector of claim 17, wherein the second promoter sequence is a H1 promoter sequence, and the third promoter sequence is a U6 promoter sequence.

20. A method for editing a nucleic acid including a target sequence, in the cell, the method comprising:
delivering a Cas12f1 protein or a nucleic acid encoding the Cas12f1 protein, and an engineered guide RNA or a nucleic acid encoding the engineered guide RNA, into a cell, so that a CRISPR/Cas12f1 complex is formed in the cell, and
the nucleic acid is edited by the CRISPR/Cas12f1 complex, wherein the engineered guide RNA comprises a scaffold sequence which interacts with the Cas12f1 protein, a spacer sequence which is complementary to a target sequence, and a U-rich tail sequence which is linked to 3′-terminus of the spacer sequence, wherein the U-rich tail sequence is represented by the formula of claim 1.

21. The method of claim 20, wherein in the delivering, the Cas12f1 protein and the engineered guide RNA are introduced in the form of a ribonucleoprotein complex into the cell.

22. The method of claim 20, wherein in the delivering, a vector including the nucleic acid encoding the Cas12f1 protein and the nucleic acid encoding the engineered guide RNA is introduced into the cell.

23. The method of claim 20, wherein the vector is one or more selected from the group consisting of a retrovirus, a lentivirus, an adenovirus, an adeno-associated virus (AAV), a vaccinia virus, a poxvirus, and a herpes simplex virus.

24-25. (canceled)

26. The engineered guide RNA of claim 6, wherein the tracrRNA sequence has a sequence of SEQ ID NO: 60, and the crRNA repeat sequence has a sequence of SEQ ID NO: 58.

27. The vector of claim 17, wherein the vector is a viral vector, a plasmid vector, or a linear PCR amplicon.

28. The vector of claim 27, wherein the vector is one selected from the group consisting of a retrovirus, a lentivirus, an adenovirus, an adeno-associated virus (AAV), a vaccinia virus, a poxvirus and a herpes simplex virus.
